(19) United States
(12) Patent Application Publication
(10) Pub. No.: US 2024/0391922 A1
(43) Pub. Date: Nov. 28, 2024

(54) PYRROLOPYRROLE COMPOSITIONS AS PYRUVATE KINASE (PKR) ACTIVATORS

(71) Applicant: Novo Nordisk Heathcare AG, Zurich (CH)

(72) Inventors: Anna Ericsson, Shrewsbury, MA (US); Neal Green, Newton, MA (US); Gary Gustafson, Hilton Head Island, SC (US); Bingsong Han, Westwood, MA (US); David R. Lancia, JR., Westwood, MA (US); Lorna Mitchell, Cambridge, MA (US); David Richard, Littleton, MA (US); Tatiana Shelekhin, Ridgefield, CT (US); Chase C. Smith, Rutland, MA (US); Zhongguo Wang, Lexington, MA (US); Xiaozhang Zheng, Lexington, MA (US)

(21) Appl. No.: 18/775,532

(22) Filed: Jul. 17, 2024

Related U.S. Application Data
(63) Continuation of application No. 18/124,071, filed on Mar. 21, 2023, now Pat. No. 12,071,440.
Application No. 18/124,071 is a continuation of application No. 17/239,364, filed on Apr. 23, 2021, now Pat. No. 11,649,242.
Application No. 17/239,364 is a continuation of application No. 16/496,279, filed on Sep. 20, 2019, now Pat. No. 11,014,927.
(60) Provisional application No. 62/473,751, filed on Mar. 20, 2017.

(51) Int. Cl. C07D 487/04; A61K 9/00; A61K 31/407; A61P 7/00; C07D 519/00

(52) U.S. Cl. CPC C07D 487/04; A61K 9/0053; A61K 31/407; A61P 7/00; C07D 519/00

(57) ABSTRACT

The disclosure relates to modulating pyruvate kinase and provides novel chemical compounds useful as activators of PKR, as well as various uses of these compounds. PKR activating compounds are useful in the treatment of diseases and disorders associated with PKR and/or PKM2, such as pyruvate kinase deficiency (PKD), sickle cell disease (SCD), and thalassemia.

### CROSS REFERENCE TO RELATED APPLICATIONS

This application is a continuation of U.S. patent application Ser. No. 18/124,071, filed Mar. 21, 2023, which is a continuation of U.S. patent application Ser. No. 17/239,364, filed Apr. 23, 2021 (Issued U.S. Ser. No. 11/649,242 on May 16, 2023), which is a continuation of U.S. patent application Ser. No. 16/496,279, filed Sep. 20, 2019 (Issued US11014927 on May 25, 2021), which is the U.S. National Phase of International Application No. PCT/US2018/023405, filed Mar. 20, 2018, which claims the benefit of U.S. Provisional Patent Application No. 62/473,751, filed on Mar. 20, 2017, each of which is incorporated by reference in its entirety.

### TECHNICAL FIELD

The present disclosure is directed to modulating pyruvate kinase, including novel compounds useful as PKR activators.

### BACKGROUND

Pyruvate Kinase (PK) converts phosphoenolpyruvate (PEP) and adenosine diphosphate (ADP) to pyruvate and adenosine triphosphate (ATP), respectively, which is the final step in glycolysis. In humans, four PK isoforms are expressed by two structural genes. The PKLR gene encodes PKR and PKL tissue specific isoforms expressed in erythroid cells and liver, respectively. The PKM gene codes for isoforms PKM1, expressed in brain and skeletal muscle, and PKM2 (M2-type pyruvate kinase), expressed in fetal and most adult tissues except erythroid cells (Takenaka et al, Eur J Biochem 1991, 198:101).

Mutations in the PKLR gene can lead to pyruvate kinase deficiency (PKD), an autosomal recessive disorder, which is the most frequent enzymatic defect of the glycolytic pathway in erythrocytes. Over 200 different mutations have been identified on the structural PKLR gene (Bianchi et al, Blood Cells Mol Dis 2000, 26:47). Generally, most PKD patients are heterozygous with two different mutant alleles, but homozygous mutations have also been described (Diez et al. Blood 2005, 106:1851). Clinical symptoms of PKD vary considerably from mild to severe anemia. Mutations can reduce PK enzymatic activity or decrease PK protein stability. Pathological manifestations are usually observed when enzyme activity falls below 25% normal PK activity, and severe disease has been associated with a high degree of reticulocytosis (Miwa et al, Am J Hematol 51:122). Although the global incidence of PKD is unknown, it has been estimated at 51 cases per million in North America (Beutler et al, Blood 2000, 95:3585).

Currently, there is no definitive treatment for severe PKD (Cazzola, Haematologica 2005, 90:1). Although splenectomy can be clinically useful in patients with severe disease, in some cases, allogeneic hematopoietic transplantation is required (Tanphaichitr et al, Bone Marrow Transplant 2000, 26:689). In these patients, hematopoietic stem cell (HSC) gene therapy might be a good and more effective treatment. Gene therapy strategies for PKD have been addressed in animal models demonstrating that introduction of the correct version of the human PKLR gene into hematopoietic stem cells using retroviral vectors alleviates the disease (Meza et al, Hum Gene Ther 2007, 18:502). Although bone marrow transplant (BMT) or gene therapy strategies would be definitive treatments of the disease, important adverse effects are associated with both approaches (Aiuti et al, Gene Ther 2007, 14:1555).

There remains a need for strategies to improve the treatment of diseases related to PKR, such as PKD, including the discovery/development of PKR activating small molecules. PKR exists in both a dimeric and tetrameric state, but functions most efficiently as a tetramer. Small molecules have been shown to be capable of shifting the equilibrium of PKR to the tetrameric (most active) form, providing a mechanistic rationale for their use as therapy for PKD-associated hemolytic anemia. Thus, there is a need for PKR activating compounds, useful for treating diseases and disorders associated with modulation of PKR and/or PKM2.

### SUMMARY

Compounds that activate PKR are disclosed herein. PKR Activating Compounds disclosed herein can increase the activity of wild-type and mutant PK enzymes in biochemical assays disclosed herein (e.g., Example 47). Data from PKR Activating Compounds herein illustrate the potential for these compounds to restore glycolytic pathway activity in patients with PK deficiency, with the goal of providing clinical benefit. Compounds disclosed herein are useful in the treatment of diseases or disorders associated with pyruvate kinase function. For example, the PKR Activating Compounds disclosed can be useful in the treatment of diseases, including but not limited to, PKD, sickle cell disease (SCD) (e.g., sickle cell anemia), and thalassemia (e.g., beta-thalassemia). In other embodiments, the compounds can be useful in the treatment of other indications related to pyruvate kinase modulation.

One aspect of the present disclosure relates to compounds of Formula I (e.g., compounds of Formula (I) identified as PKR Activating Compounds using the Luminescence Assay Protocol of Example 47):

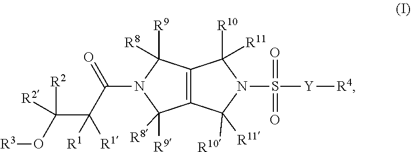

and pharmaceutically acceptable salts thereof,
wherein:
Y is a bond, —(CR⁵R^{5′})ₜ—, —NR⁵(CR⁵R^{5′})ₜ—, or —O—;
each R¹, R^{1′}, R², and R^{2′} is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R³, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
or R¹ and R^{1′}, or R² and R^{2′}, together with the atom to which they are attached, can combine to form a —(C₃-C₈)cycloalkyl ring, heterocycle, (C₅-C₈)spirocycle or 5- to 8-membered spiroheterocycle;
or R¹ and R², together with the atoms to which they are attached, can combine to form a —(C₃-C₈)cycloalkyl or a 3- to 8-membered heterocycle;
R³ is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, —S(O)₂R⁵, —S(O)₂NR⁵R^{5′}, —S(O)R⁵, —S(O)NR⁵R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
or R² and R³, together with the atoms to which they are attached, can combine to form a 5- to 8-membered heterocyclic ring;
or R¹ and R³, together with the atoms to which they are attached, can combine to form a 5- to 8-membered heterocyclic ring;
R⁴ is —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
each R⁵ and R^{5′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, or —C(O)OR⁶, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁶, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, and —C(O)OR⁶;
or two R⁵ on adjacent atoms together with the atoms to which they are attached form an aryl ring optionally substituted with one or more R⁶; or two R⁵ on adjacent atoms together with the atoms to which they are attached form a heteroaryl ring optionally substituted with one or more R⁶; or two R⁵ on adjacent atoms together with the atoms to which they are attached form a (C₃-C₈)cycloalkyl ring optionally substituted with one or more R⁶; or two R⁵ on adjacent atoms together with the atoms to which they are attached form a heterocycloalkyl ring optionally substituted with one or more R⁶;
or two R^{5′} on adjacent atoms together with the atoms to which they are attached form an aryl ring optionally substituted with one or more R⁶; or two R^{5′} on adjacent atoms together with the atoms to which they are attached form a heteroaryl ring optionally substituted with one or more R⁶; or two R^{5′} on adjacent atoms together with the atoms to which they are attached form a (C₃-C₈)cycloalkyl ring optionally substituted with one or more R⁶; or two R^{5′} on adjacent atoms together with the atoms to which they are attached form a heterocycloalkyl ring optionally substituted with one or more R⁶;
each R⁶ and R^{6′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, or —C(O)OR⁷, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁷, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, and —C(O)OR⁷;
each R⁷ and R^{7′} is independently, at each occurrence, —H, —(C₁-C₆)alkyl, —(C₂-C₆)alkenyl, —(C₂-C₆)alkynyl, —(C₃-C₈)cycloalkyl, —(C₄-C₅)cycloalkenyl, heterocyclyl, aryl, heteroaryl, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, or —C(O)OH, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, and —C(O)OH;
each R⁸, R^{8′}, R⁹, R^{9′}, R¹⁰, R^{10′}, R¹¹, and R^{11′} is independently, at each occurrence, —H, —(C₁-C₆)alkyl, —(C₂-C₆)alkenyl, —(C₂-C₆)alkynyl, —(C₃-C₈)cycloalkyl, or —(C₄-C₈)cycloalkenyl, wherein each alkyl, alkenyl, alkynyl, cycloalkyl and cycloalkenyl, is optionally substituted with one or more substituents selected from the group consisting of oxo, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, or and —C(O)OH; and
t is 0, 1, 2, or 3.

Unless otherwise indicated herein, each occurrence of R⁷ and R^{7′} disclosed herein for each of R⁶, R^{6′}, R⁸, R^{8′}, R⁹, R^{9′}, R¹⁰, R^{10′}, R¹¹, and R^{11′} is independently selected from any of the possible recited values of R⁷ and R^{7′}. For example, the value R⁷ may have a different value for each of R⁶, R^{6′}, R⁸, R^{8′}, R⁹, R^{9′}, R¹⁰, R^{10′}, R¹¹, and R^{11′} unless otherwise indicated herein.

The present disclosure also provides compounds of Formula (I) and pharmaceutically acceptable salts thereof, wherein:
Y is a bond;
R¹ is selected from the group consisting of —H, —(C₁-C₆)alkyl, 6-membered aryl, and 6-membered heteroaryl;
R^{1′} is selected from the group consisting of —H and —(C₁-C₆)alkyl;
or R¹ and R^{1′}, together with the atom to which they are attached, can combine to form a —(C₃-C₈)cycloalkyl or a 3- to 8-membered heterocycle;
each R² and R^{2′} is independently selected from the group consisting of —H and —(C₁-C₆)alkyl;
R³ is —H or —(C₁-C₆)alkyl;
or R¹ and R³, together with the atoms to which they are attached, can combine to form a 5-to 8-membered heterocyclic ring, optionally fused to an aryl or heteroaryl ring;
R⁴ is 6- to 10-membered aryl or 6- to 10-membered heteroaryl, each optionally substituted with one or more substituents selected from the group consisting of oxo, halogen, —CN, —R⁵, —OR⁵, or —NR⁵R^{5′};
each R⁵ and R^{5′} is independently, at each occurrence, —H, —(C₁-C₆)alkyl optionally substituted with one or more halogen, —OR⁶, or —NR⁶R^{6′};
or two R⁵ on adjacent atoms of R⁴ together with the atoms to which they are attached optionally form a 5- or 6-membered heterocycloalkyl ring optionally substituted with one or more R⁶;
each R⁶ is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, or —C(O)OR⁷, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁷, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR'S(O)R^{7′}, —C(O)R⁷, and —C(O)OR⁷;
each R⁷ and R^{7′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, or —C(O)OH, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, and —C(O)OH;
each R⁸, R^{8′}, R⁹, R^{9′}, R¹⁰, R^{10′}, R¹¹, and R^{11′} is independently, at each occurrence, —H or —(C₁-C₆)alkyl optionally substituted with one or more substituents selected from the group consisting of oxo, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, or and —C(O)OH.

For example, the present disclosure relates to compounds of Formula I and pharmaceutically acceptable salts thereof, wherein:
Y is a bond;
each R¹ and R^{1′} is independently selected from the group consisting of —H, —(C₁-C₆)alkyl, aryl, and heteroaryl, wherein each alkyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
or R¹ and R^{1′}, together with the atom to which they are attached, can combine to form a —(C₃-C₅)cycloalkyl ring, heterocycle, (C₅-C₈)spirocycle or 5- to 8-membered spiroheterocycle;
each R² and R^{2′} is independently selected from the group consisting of —H and —(C₁-C₆)alkyl, optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
R³ is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, —S(O)₂R⁵, —S(O)₂NR⁵R^{5′}, —S(O)R⁵, —S(O)NR⁵R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
or R¹ and R³, together with the atoms to which they are attached, can combine to form a 5- to 8-membered heterocyclic ring, optionally fused to an aryl or heteroaryl ring;
R⁴ is 6- to 10-membered aryl or 6- to 10-membered heteroaryl, each optionally substituted with one or more substituents selected from the group consisting of oxo, halogen, —CN, —R⁵, —OR⁵ or —NR⁵R^{5′};
each R⁵ and R^{5′} is independently, at each occurrence, —H, —(C₁-C₆)alkyl, —OR⁶, or —NR⁶R^{6′};
or any two R⁵ on adjacent atoms of R⁴, together with the atoms to which they are attached form a 5- or 6-membered heterocycloalkyl ring optionally substituted with one or more R⁶;
each R⁶ is independently, at each occurrence, —H or —(C₁-C₆)alkyl; and
each R⁸, R^{8′}, R⁹, R^{9′}, R¹⁰, R^{10′}, R¹¹, and R^{11′} is independently, at each occurrence, —H.

In some PKR Activating Compounds, R⁴ is 6-membered aryl or heteroaryl substituted with two —R⁵, selected from the group consisting of —OR⁶ and —NR⁶R^{6′}, on adjacent atoms of R⁴, that together with the atoms to which they are attached form a heterocycloalkyl ring fused to R⁴ that is optionally substituted with one or more R⁶, selected from the group consisting of —H and —(C₁-C₆)alkyl.

In another aspect, the disclosure provides pharmaceutical compositions comprising a compound of Formula I and a pharmaceutically acceptable carrier.

In another aspect, the disclosure provides methods of treating a disease or disorder associated with modulation of pyruvate kinase (PKR) which comprises administering to a patient in need thereof an effective amount of a compound of Formula I.

The present disclosure also provides methods of treating a disease associated with decreased activity of PKR in a subject in need thereof which comprises administering to the subject an effective amount of a compound of Formula I.

Another aspect of the present disclosure is a method of activating PKR, comprising contacting PKR with an effective amount of a compound of Formula I.

Further aspects of the present disclosure include: methods of increasing the lifetime of red blood cells; methods of regulating 2,3-diphosphoglycerate levels in blood; and methods of regulating ATP levels in blood; each of the foregoing methods comprising administering to a subject in need thereof an effective amount of a compound of Formula I.

Another aspect of the present disclosure provides methods of treating hereditary non-spherocytic hemolytic anemia comprising administering to a subject in need thereof an effective amount of a compound of Formula I.

Also provided herein are methods of treating a disease or disorder associated with increased 2,3 diphosphoglycerate levels comprising administering to a subject in need thereof an effective amount of a compound of Formula I.

Another aspect of the disclosure provided herein includes methods of treating a disease or disorder associated with decreased ATP levels comprising administering to a subject in need thereof an effective amount of a compound of Formula I.

A further aspect of the present disclosure includes methods of treating sickle cell anemia comprising administering to a subject in need thereof a therapeutically effective amount of any of Formula I.

A further aspect of the present disclosure includes methods of treating hemolytic anemia comprising administering to a subject in need thereof a therapeutically effective amount of a compound of Formula I.

Another aspect of the present disclosure includes methods of treating beta thalassemia comprising administering to a subject in need thereof a therapeutically effective amount of a compound of Formula I.

### BRIEF DESCRIPTION OF THF DRAWING

FIG. 1 shows an exemplary dose-response curve for compounds disclosed herein. Dose-response curves may be generated using the standard four parameter fit algorithm of ActivityBase XE Runner to determine MAX % Fold, MIN % Fold, slope and AC₅₀. MAX % Fold is the highest % fold increase observed at any concentration of compound, and MIN % Fold is the lowest % fold increase observed at any concentration of compound. The AC₅₀ value for a compound is the concentration (μM) corresponding to the midway between the maximum and minimum values of the four parameter logistic curve fit (i.e., at which the % fold increase along the four parameter logistic curve fit is halfway between MAX % Fold and MIN % Fold (% Fold Midpoint). Another useful parameter for evaluating compounds of this disclosure is % Fold@1.54 μM, which is the % fold increase at a compound concentration of 1.5 μM (e.g., 1.54 μM). X-axis and y-axis not necessarily to scale.

### DETAILED DESCRIPTION

The present disclosure relates to compounds and compositions that are capable of activating the activity of PKR and/or PKM2. The disclosure features methods of treating a disease or disorder in which PKR and/or PKM2 plays a role by administering to a patient in need thereof a therapeutically effective amount of a compound of Formula (I), or a pharmaceutically acceptable salt thereof. The methods of the present disclosure can be used in the treatment of a variety of PKR and/or PKM2 dependent diseases and disorders by activating the activity of PKR and/or PKM2 enzymes. Activation of PKR and PKM2 provides a novel approach to the treatment of diseases including, but not limited to, PKD, SCD (e.g., sickle cell anemia), and thalassemia (e.g., beta-thalassemia. In some embodiments, the PKR Activating Compounds disclosed herein can be useful for the treatment of hereditary blood disorders related to pyruvate kinase activity, including PKD and SCD.

In a first aspect of the disclosure, compounds of Formula (I) are described:

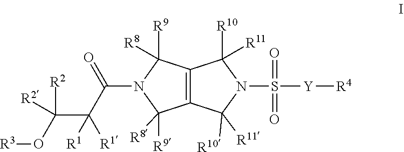

and pharmaceutically acceptable salts, thereof, wherein Y, R¹, R^{1′}, R², R^{2′}, R³, R⁴, R⁸, R^{8′}, R⁹, R^{9′}, R¹⁰, R^{10′}, R¹¹, and R^{11′} are as described herein above.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (e.g., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (e.g., a pure hydrocarbon). Alternatively, the same optionally substituted alkyl group can have substituents in place of one or more hydrogen atoms. For instance, it can, at any point along the chain be bounded to a halogen atom, a hydroxyl group, or any other substituent described herein. Thus the term “optionally substituted” means that a given chemical moiety has the potential to contain other functional groups, but does not necessarily have any further functional groups. Suitable substituents used in the optional substitution of the described groups include, without limitation, halogen, oxo, —OH, —CN, —COOH, —CH₂CN, —O—(C₁-C₆)alkyl, (C₁-C₆)alkyl, (C₁-C₆)alkoxy, (C₁-C₆)haloalkyl, (C₁-C₆)haloalkoxy, —O—(C₂-C₆)alkenyl, —O—(C₂-C₆)alkynyl, (C₂-C₆)alkenyl, (C₂-C₆)alkynyl, —OP(O)(OH)₂, —OC(O)(C₁-C₆)alkyl, —C(O)C₁-C₆)alkyl, —OC(O)O(C₁-C₆)alkyl, —NH₂, —NH((C₁-C₆)alkyl), —N((C₁-C₆)alkyl)₂, —NHC(O)C₁-C₆)alkyl, —C(O)NH(C₁-C₆)alkyl, —S(O)₂(C₁-C₆)alkyl, —S(O)NH(C₁-C₆)alkyl, and S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Unless otherwise specifically defined, the term “aryl” refers to cyclic, aromatic hydrocarbon groups that have 1 to 3 aromatic rings having a total of 5 to 14 ring atoms, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, -halogen, —O—(C₁-C₆)alkyl, (C₁-C₆)alkyl, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, NH((C₁-C₆)alkyl), N((C₁-C₆)alkyl)₂, —S(O)₂—(C₁-C₆)alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have an unsaturated or partially saturated ring fused with a fully unsaturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or polycyclic aromatic radical of 5 to 24 ring atoms, containing one or more ring heteroatoms selected from the group consisting of N, O, and S, the remaining ring atoms being C. Heteroaryl as herein defined also means a bicyclic heteroaromatic group wherein the heteroatom is selected from the group consisting of N, O, and S. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two fused rings the heteroaryl groups herein defined may have an unsaturated or partially saturated ring fused with a fully unsaturated ring. Exemplary ring systems of these heteroaryl groups include indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuran, indolinyl, indolyl, and dihydrobenzoxanyl.

“Halogen” or “halo” refers to fluorine, chlorine, bromine, or iodine.

“Alkyl” refers to a straight or branched chain saturated hydrocarbon containing 1-12 carbon atoms. Examples of a (C₁-C₆)alkyl group include, but are not limited to, methyl, ethyl, propyl, butyl, pentyl, hexyl, isopropyl, isobutyl, sec-butyl, tert-butyl, isopentyl, neopentyl, and isohexyl. An alkyl group may be substituted by one or more substituents.

“Alkoxy” refers to a straight or branched chain saturated hydrocarbon containing 1-12 carbon atoms containing a terminal “0” in the chain, e.g., —O(alkyl). Examples of alkoxy groups include without limitation, methoxy, ethoxy, propoxy, butoxy, t-butoxy, or pentoxy groups.

The term “alkylene” or “alkylenyl” refers to a divalent alkyl radical. Any of the above mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” or “carbocyclyl” means monocyclic or polycyclic saturated rings containing 3-18 carbon atoms. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl and derivatives thereof. A C₃-C₈ cycloalkyl is a cycloalkyl group containing between 3 and 8 carbon atoms. A cycloalkyl group can be fused (e.g., decalin) or bridged (e.g., norbornane). A cycloalkyl group may be substituted by one or more substituents.

“Heterocyclyl” or “heterocycloalkyl” means 5- to 7-membered monocyclic or 7- to 10-membered polycyclic rings containing carbon and heteroatoms taken from oxygen, nitrogen, or sulfur, where such rings are either saturated or partially unsaturated. The heterocycloalkyl ring structure may be substituted by one or more substituents. The substituents can themselves be optionally substituted. Examples of heterocyclyl rings include, but are not limited to, oxetanyl, azetadinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “hydroxyalkyl” means an alkyl group as defined above, where the alkyl group is substituted with one or more OH groups. Examples of hydroxyalkyl groups include HO—CH₂—, HO—CH₂—CH₂— and CH₃—CH(OH)—.

The term “haloalkyl” as used herein refers to an alkyl group, as defined herein, which is substituted with one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted with one or more halogen. Examples of haloalkoxy groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., —C≡N.

“Spirocycloalkyl” or “spirocyclyl” means carbogenic bicyclic ring systems with both rings connected through a single atom. The ring can be different in size and nature, or identical in size and nature. Examples include spiropentane, spirohexane, spiroheptane, spirooctane, spirononane, or spirodecane. One or both of the rings in a spirocycle can be fused to another carbocyclic, heterocyclic, aromatic, or heteroaromatic ring. A (C₅-C₁₂)spirocycloalkyl is a spirocycle containing between 5 and 12 carbon atoms. One or more of the carbon atoms can be substituted with a heteroatom.

The term “spiroheterocycloalkyl” or “spiroheterocyclyl” is understood to mean a spirocycle wherein at least one of the rings is a heterocycle (e.g., at least one of the rings is furanyl, morpholinyl, or piperadinyl).

The term “isomer” refers to compounds that have the same composition and molecular weight but differ in physical and/or chemical properties. The structural difference may be in constitution (e.g., geometric isomers) or in the ability to rotate a plane of polarized light (stereoisomers). With regard to stereoisomers, the compounds of Formula (I) may have one or more asymmetric carbon atoms and may occur as racemates, racemic mixtures or as individual enantiomers or diastereomers.

The disclosure also includes pharmaceutical compositions comprising an effective amount of a disclosed compound and a pharmaceutically acceptable carrier.

“Pharmaceutically acceptable salts” are well known in the art. For example, S. M. Berge et al., describe pharmaceutically acceptable salts in detail in J. Pharmaceutical Sciences, 1977, 66, 1-19, incorporated herein by reference. Representative pharmaceutically acceptable salts include, e.g., water-soluble and water-insoluble salts, such as acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. The compounds of Formula I may form salts which are also within the scope of this disclosure. Reference to a compound of Formula I herein is understood to include reference to salts thereof, unless otherwise indicated.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent, or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound, a pharmaceutically acceptable salt of a disclosed compound or a composition to a subject, a pharmaceutically acceptable salt of a compound, or a composition to a subject, which can form an equivalent amount of active compound within the subject's body.

The term “cancer” includes, but is not limited to, the following cancers: bladder cancer, breast cancer (e.g., ductal carcinoma), cervical cancer (e.g., squamous cell carcinoma), colorectal cancer (e.g., adenocarcinoma), esophageal cancer (e.g., squamous cell carcinoma), gastric cancer (e.g., adenocarcinoma, medulloblastoma, colon cancer, choriocarcinoma, squamous cell carcinoma), head and neck cancer, hematologic cancer (e.g., acute lymphocytic anemia, acute myeloid leukemia, acute lymphoblastic B cell leukemia, anaplastic large cell lymphoma, B-cell lymphoma, Burkitt's lymphoma, chronic lymphocytic leukemia, chronic eosinophillic leukemia/hypereosinophillic syndrome, chronic myeloid leukemia, Hodgkin's lymphoma, mantle cell lymphoma, multiple myeloma, T-cell acute lymphoblastic leukemia), lung cancer (e.g., bronchioloalveolar adenocarcinoma, mesothelioma, mucoepidermoid carcinoma, small-cell lung cancer, non-small cell lung cancer, adenocarcinoma, squamous cell carcinoma), liver cancer (e.g., hepatocellular carcinoma), lymphoma, neurological cancer (e.g., glioblastoma, neuroblastoma, neuroglioma), ovarian (e.g., adenocarcinoma), pancreatic cancer (e.g., ductal carcinoma), prostate cancer (e.g., adenocarcinoma), renal cancer (e.g., renal cell carcinoma, clear cell renal carcinoma), sarcoma (e.g., chondrosarcoma, Ewings sarcoma, fibrosarcoma, multipotential sarcoma, osteosarcoma, rhabdomyosarcoma, synovial sarcoma), skin cancer (e.g., melanoma, epidermoid carcinoma, squamous cell carcinoma), thyroid cancer (e.g., medullary carcinoma), and uterine cancer.

Unless otherwise indicated, “PKR Activating Compound” as used herein refers to a compound having one or more of the following characteristics when tested according to the Luminescence Assay Protocol of Example 47 below: (1) an AC₅₀ value of less than 40 μM; (2) a maximum % Fold (MAX % Fold) value of greater than 75%; and/or (3) a % Fold value at 1.54 μM compound concentration (% Fold@1.54 μM) of at least 75%. In some embodiments, the Luminescence Assay Protocol of Example 47 is performed with wild type (wt) PKR, G332S mutant form of PKR or R510Q mutant form of PKR. In some embodiments, the PKR Activating Compound is a compound of Formula (I). In some embodiments, the PKR Activating Compound has: (1) an AC₅₀ value of less than 0.1 μM, 0.1-1.0 μM, or 1.01-40 μM; (2) a MAX % Fold of 75% 250%, 251-500%, or 75%-500%; and/or (3) a % Fold@1.54 μM of 75%-250%, 251-500%, or 75%-500%. In some embodiments, a PKR Activating Compound has (1) an AC₅₀ value of less than 1.0 μM; (2) a MAX % Fold of 75%-500%; and/or (3) a % Fold@1.54 μM of 75%-500%. In some embodiments, a PKR Activating Compound has (1) an AC₅₀ value of less than 1.0 μM; (2) a MAX % Fold of 75%-500%; and/or (3) a % Fold@1.54 μM of 75%-500%, obtained in the Luminescence Assay Protocol with any one or more of wild type PKR (wt), G332S mutant form of PKR, or R510Q mutant form of PKR. In some embodiments, the PKR Activating Compound has (1) an AC₅₀ value of less than 1.0 μM; (2) a MAX % Fold of 75%-500%; and/or (3) a % Fold@1.54 μM of 75%-500%, obtained in the Luminescence Assay Protocol with wild type PKR (wt). In some embodiments, the PKR Activating Compound has (1) an AC₅₀ value of less than 1.0 μM; (2) a MAX % Fold of 75%-500%; and/or (3) a % Fold@1.54 μM of 75%-500%, obtained in the Luminescence Assay Protocol with any one or both of G332S mutant form of PKR or R510Q mutant form of PKR.

It should be understood that all stereoisomeric forms are included within the present disclosure, including mixtures thereof.

The compounds of the disclosure may contain asymmetric or chiral centers, and, therefore, exist in different stereoisomeric forms. It is intended that all stereoisomeric forms of the compounds of the disclosure, such as those which may exist due to asymmetric carbons on various substituents, including enantiomeric forms (which may exist even in the absence of asymmetric carbons), rotameric forms, atropisomers, and diastereomeric forms, as well as mixtures thereof, including racemic mixtures, form part of the present disclosure. The assay results may reflect the data collected for the racemic form, the enantiomerically pure form, or any other form in terms of stereochemistry. Individual stereoisomers of the compounds of the disclosure may, for example, be substantially free of other isomers, or may be admixed, for example, as racemates or with all other, or other selected, stereoisomers. In some embodiments of the disclosure, the compounds of Formula (I) are enantiomers. In some embodiments, the compounds are the (S)-enantiomer. In other embodiments the compounds are the (R)-enantiomer. In some embodiments, the compounds of Formula (I) may be (+) or (−) enantiomers.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the disclosure may be atropisomers (e.g., substituted biaryls) and are considered as part of this disclosure. Enantiomers can also be separated by use of a chiral HPLC column.

In addition, unless otherwise indicated, the present disclosure embraces all geometric and positional isomers (such as, for example, 4-pyridyl and 3-pyridyl). For example, if a compound of the disclosure incorporates a double bond or a fused ring, both the cis- and trans forms, as well as mixtures, are embraced within the scope of the disclosure. If the compound contains a double bond, the substituent may be in the E or Z configuration, unless otherwise indicated. If the compound contains a disubstituted cycloalkyl, the cycloalkyl substituent may have a cis- or trans configuration, unless otherwise indicated.

Compounds of the disclosure, and pharmaceutically acceptable salts and stereoisomers, thereof may exist in their tautomeric form (for example, as an amide or imino ether). Moreover, all keto-enol and imine-enamine forms of the compounds are included in the disclosure. All such tautomeric forms are contemplated herein as part of the present disclosure.

The use of the terms “salt” and the like, is intended to equally apply to the salt of enantiomers, stereoisomers, rotamers, tautomers, positional isomers, and racemates of the inventive compounds.

The present disclosure relates to compounds or pharmaceutically acceptable salts thereof, capable of activating PKR and/or PKM2, which are useful for the treatment of diseases and disorders associated with modulation of a PKR and/or PKM2 enzyme. The disclosure further relates to compounds, or pharmaceutically acceptable salts thereof, which are useful for activating PKR and/or PKM2.

### Compounds of the Disclosure

In one aspect of the disclosure, compounds of Formula (I) are provided:

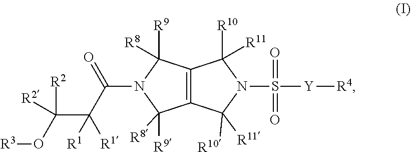

and pharmaceutically acceptable salts thereof, where Y, R¹, R^{1′}, R², R^{2′}, R³, R⁴, R⁸, R^{8′}, R⁹, R^{9′}, R¹⁰, R^{10′}, R¹¹, and R^{11′} are as defined above and described in classes and subclasses herein, both singly and in combination.

Unless otherwise indicated herein, each occurrence of R⁷ and R^{7′} disclosed herein for each of R⁶, R^{6′}, R⁸, R^{8′}, R⁹, R^{9′}, R¹⁰, R^{10′}, R¹¹, and R^{11′} is independently selected from any of the possible recited values of R⁷ and R^{7′}. For example, the value R⁷ may have a different for each of R⁶, R^{6′}, R⁸, R^{8′}, R⁹, R^{9′}, R¹⁰, R^{10′}, R¹¹, and R^{11′} unless otherwise indicated herein.

In some embodiments, the compounds of Formula I have an AC₅₀ value≤40 μM for PKR activity determined by a luminescence assay (e.g., that described in Example 47, below). In some embodiments, the compounds of Formula I have an AC₅₀ value ≤1.0 μM for PKR activity determined by a luminescence assay (e.g., that described in Example 47, below). In some embodiments, the compounds of Formula I have an AC₅₀ value≤0.1 μM for PKR activity determined by a luminescence assay (e.g., that described in Example 47, below).

In some embodiments, the compounds of Formula I are of the Formula (Ia):

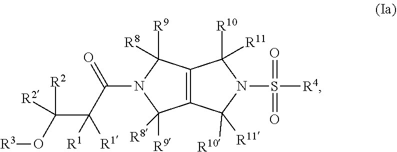

and pharmaceutically acceptable salts thereof, wherein R¹, R^{1′}, R², R^{2′}, R³, R⁴, R⁸, R^{8′}, R⁹, R^{9′}, R¹⁰, R^{10′}, R¹¹, and R^{11′} are as defined above and described in classes and subclasses herein, both singly and in combination.

In some embodiments, the compounds of Formula I are of the Formula (Ib):

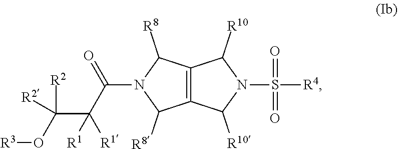

and pharmaceutically acceptable salts thereof, wherein R¹, R^{1′}, R², R^{2′}, R³, R⁴, R⁸, R^{8′}, R¹⁰, and R^{10′} are as defined above and described in classes and subclasses herein, both singly and in combination.

In some embodiments, the compounds of Formula I are of the Formula (Ic):

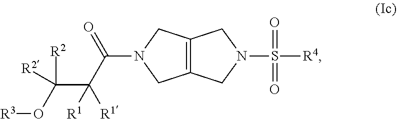

and pharmaceutically acceptable salts thereof, wherein R¹, R^{1′}, R², R^{2′}, R³, and R⁴ are as defined above and described in classes and subclasses herein, both singly and in combination.

In some embodiments, compounds of Formula (Ic) are provided, wherein:
each R¹, R^{1′}, R², and R^{2′} is independently —H, —(C₁-C₆)alkyl, aryl, or heteroaryl, wherein each alkyl, aryl, or heteroaryl is optionally substituted with one or more —OR⁵;
or R¹ and R^{1′}, together with the atom to which they are attached, can combine to form a —(C₃-C₈)cycloalkyl ring;
R³ is —H or —(C₁-C₆)alkyl;
or R¹ and R³, together with the atoms to which they are attached, can combine to form a 5- to 8-membered heterocyclic ring, optionally fused to an aryl ring;
R⁴ is aryl or heteroaryl, wherein each aryl or heteroaryl is optionally substituted with one or more substituents selected from the group consisting of —R⁵ and —OR⁵;
each R⁵ is independently —H or —(C₁-C₆)alkyl, wherein each alkyl is optionally substituted with one or more halogen;
or two R⁵ on adjacent atoms together with the atoms to which they are attached form a heterocycloalkyl ring optionally substituted with one or more R⁶; and
each R⁶ is —(C₁-C₆)alkyl.

In some embodiments, compounds of Formula (Ic) are provided, wherein:
each R¹, R^{1′}, R², and R^{2′} is independently —H, phenyl, pyridyl, ethyl, or methyl optionally substituted with —OR⁵;
or R¹ and R^{1′}, together with the atom to which they are attached, can combine to form a cyclopropyl ring;
R³ is —H or methyl;
or R¹ and R³, together with the atoms to which they are attached, can combine to form a tetrahydrofuran, tetrahydropyran, 2,3-dihydrobenzofuran, or morpholine;
R⁴ is phenyl, pyridyl, benzothiazolyl, benzofuranyl, or benzoxazolyl, wherein each phenyl, pyridyl, or benzoxazolyl is optionally substituted with one or two substituents selected from the group consisting of —R⁵ and —OR⁵;
each R⁵ is independently —H or methyl optionally substituted with two or more halogen;
or two R⁵ on adjacent atoms together with the atoms to which they are attached form a heterocycloalkyl ring, comprising two heteroatoms selected from the group consisting of 0 and N, optionally substituted with one or two R⁶; and
each R⁶ is methyl.

In some embodiments, the compounds of Formula I are of the Formula (Id-1):

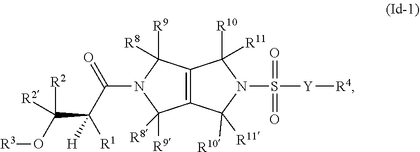

and pharmaceutically acceptable salts thereof, wherein Y, R⁴, R⁸, R^{8′}, R⁹, R^{9′}, R¹⁰, R^{10′}, R¹¹, and R^{11′} are as defined above and described in classes and subclasses herein, both singly and in combination; and
R¹ is —(C₁-C₆)alkyl, —(C₂-C₆)alkenyl, —(C₂-C₆)alkynyl, —(C₃-C₅)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
each R² and R^{2′} is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
or R² and R^{2′}, together with the atom to which they are attached, can combine to form —(C₃-C₈)cycloalkyl ring, heterocycle, (C₅-C₈)spirocycle or 5- to 8-membered spiroheterocycle;
or R¹ and R², together with the atoms to which they are attached, can combine to form a —(C₃-C₈)cycloalkyl or a 3- to 8-membered heterocycle;
R³ is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, —S(O)₂R⁵, —S(O)₂NR⁵R^{5′}, —S(O)R⁵, —S(O)NR⁵R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
or R² and R³, together with the atoms to which they are attached, can combine to form a 5- to 8-membered heterocyclic ring; and
or R¹ and R³, together with the atoms to which they are attached, can combine to form a 5- to 8-membered heterocyclic ring, optionally fused to an aryl or heteroaryl ring.

In some embodiments, the compounds of Formula (Id-1) are of the Formula (Ia-1):

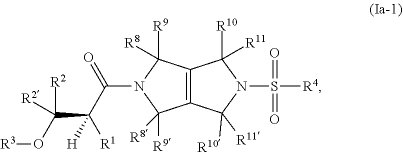

and pharmaceutically acceptable salts thereof, wherein R¹, R², R^{2′}, R³, R⁴, R⁸, R^{8′}, R⁹, R^{9′}, R¹⁰, R^{10′}, R¹¹, and R^{11′} are as defined above and described in classes and subclasses herein, both singly and in combination.

In some embodiments, the compounds of Formula (Id-1) are of the Formula (Ib-1):

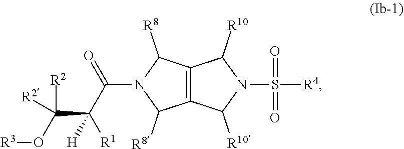

and pharmaceutically acceptable salts thereof, wherein R¹, R², R^{2′}, R³, R⁴, R⁸, R^{8′}, R¹⁰, and R^{10′} are as defined above and described in classes and subclasses herein, both singly and in combination.

In some embodiments, the compounds of Formula (Id-1) are of the Formula (Ic-1):

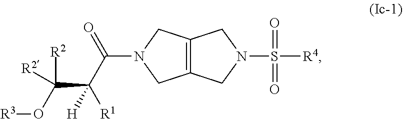

and pharmaceutically acceptable salts thereof, wherein R¹, R², R^{2′}, R³, and R⁴ are as defined above and described in classes and subclasses herein, both singly and in combination.

In some embodiments, the compounds of Formula I are of the Formula (Id-2):

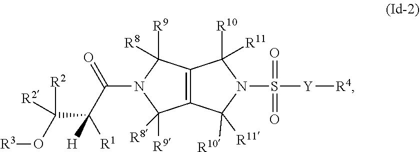

and pharmaceutically acceptable salts, thereof, wherein Y, R⁴, R⁸, R^{8′}, R⁹, R^{9′}, R¹⁰, R^{10′}, R¹¹, and R^{11′} are as defined above and described in classes and subclasses herein, both singly and in combination; and
R¹ is —(C₁-C₆)alkyl, —(C₂-C₆)alkenyl, —(C₂-C₆)alkynyl, —(C₃-C₅)cycloalkyl, —(C₄-C₅)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
each R² and R^{2′} is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
or R² and R^{2′}, together with the atom to which they are attached, can combine to form —(C₃-C₅)cycloalkyl ring, heterocycle, (C₅-C₈)spirocycle or 5- to 8-membered spiroheterocycle;
or R¹ and R², together with the atoms to which they are attached, can combine to form a —(C₃-C₅)cycloalkyl or a 3- to 8-membered heterocycle;
R³ is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, —S(O)₂R⁵, —S(O)₂NR⁵R^{5′}, —S(O)R⁵, —S(O)NR⁵R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
or R² and R³, together with the atoms to which they are attached, can combine to form a 5- to 8-membered heterocyclic ring; and
or R¹ and R³, together with the atoms to which they are attached, can combine to form a 5- to 8-membered heterocyclic ring, optionally fused to an aryl or heteroaryl ring.

In some embodiments, the compounds of Formula (Id-2) are of the Formula (Ia-2):

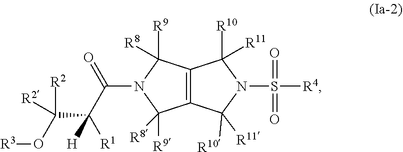

and pharmaceutically acceptable salts thereof, wherein R¹, R², R^{2′}, R³, R⁴, R⁸, R^{8′}, R⁹, R^{9′}, R¹⁰, R^{10′}, R¹¹, and R^{11′} are as defined above and described in classes and subclasses herein, both singly and in combination.

In some the Formula (Ib-2):

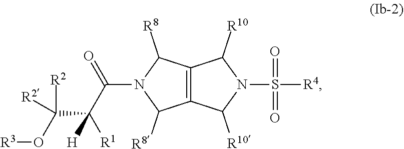

and pharmaceutically acceptable salts thereof, wherein R¹, R², R^{2′}, R³, R⁴, R⁸, R^{8′}, R¹⁰ and R^{10′} are as defined above and described in classes and subclasses herein, both singly and in combination.

In some embodiments, the compounds of Formula (Id-2) are of the Formula (Ic-2):

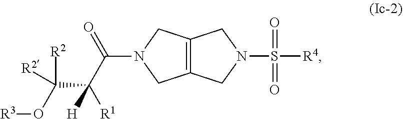

and pharmaceutically acceptable salts thereof, wherein R¹, R², R^{2′}, R³, and R⁴ are as defined above and described in classes and subclasses herein, both singly and in combination.

In some embodiments of Formula (I), (Ia), (Ib), and (Ic), R¹ and R^{1′} are each independently hydrogen, optionally substituted —(C₁-C₆)alkyl (e.g., methyl optionally substituted with —OR⁷, or ethyl), optionally substituted aryl (e.g., phenyl), or optionally substituted heteroaryl (e.g., pyridyl), or R¹ and R^{1′} are taken together with the atoms to which they are attached to form an optionally substituted —(C₃-C₄)cycloalkyl (e.g., cyclopropyl). In some embodiments, R¹ and R^{1′} are both hydrogen. In some embodiments, R¹ and R^{1′} are both optionally substituted —(C₁-C₆)alkyl. In some embodiments, one of R¹ and R^{1′} is optionally substituted —(C₁-C₆)alkyl, optionally substituted aryl, or optionally substituted heteroaryl. In some embodiments, one of R¹ and R^{1′} is optionally substituted aryl or optionally substituted heteroaryl. In some embodiments, one of R¹ and R^{1′} is hydrogen. In some embodiments, one of R¹ and R^{1′} is optionally substituted —(C₁-C₆)alkyl. In some embodiments, one of R¹ and R^{1′} is optionally substituted aryl. In some embodiments, one of R¹ and R^{1′} is optionally substituted heteroaryl. In some embodiments, R¹ and R^{1′} are taken together with the atoms to which they are attached to form an optionally substituted —(C₃-C₄)cycloalkyl.

In some embodiments of Formula (Id-1), (Ia-1), (Ib-1), (Ic-1), (Id-2), (Ia-2), (Ib-2), and (Ic-2), R¹ is optionally substituted —(C₁-C₆)alkyl (e.g., methyl optionally substituted with —OR⁵, or ethyl), optionally substituted aryl (e.g., phenyl), or optionally substituted heteroaryl (e.g., pyridyl). In some embodiments, R¹ is optionally substituted —(C₁-C₆)alkyl. In some embodiments, R¹ is optionally substituted aryl. In some embodiments, R¹ is optionally substituted heteroaryl.

In some embodiments of Formula (I), (Ia), (Ib), (Ic), (Id-1), (Ia-1), (Ib-1), (Ic-1), (Id-2), (Ia-2), (Ib-2), and (Ic-2), R² and R^{2′} are each independently hydrogen or optionally substituted —(C₁-C₆)alkyl (e.g., methyl). In some embodiments, R² and R^{2′} are both hydrogen. In some embodiments, R² and R^{2′} are both optionally substituted —(C₁-C₆)alkyl. In some embodiments, one of R² and R^{2′} is hydrogen. In some embodiments, one of R² and R^{2′} is optionally substituted —(C₁-C₆)alkyl.

In some embodiments of Formula (I), (Ia), (Ib), (Ic), (Id-1), (Ia-1), (Ib-1), (Ic-1), (Id-2), (Ia-2), (Ib-2), and (Ic-2), R³ is hydrogen or optionally substituted —(C₁-C₆)alkyl (e.g., methyl). In some embodiments, R³ is hydrogen. In some embodiments, R³ is optionally substituted —(C₁-C₆)alkyl.

In some embodiments of Formula (I), (Ia), (Ib), and (Ic), R³ and one of R¹ or R^{1′} are taken together with the atoms to which they are attached to form an optionally substituted 5-6-membered heterocyclic ring, optionally fused to an aryl ring (e.g., tetrahydrofuran, tetrahydropyran, 2,3-dihydrobenzofuran, or morpholine). In some embodiments, R³ and R¹ combine to form an optionally substituted heterocyclic ring selected from the group consisting of teterahydrofuran, tetrahydropyran, morpholine, dioxane, and 2,3-dihydrobenzofuran.

In some embodiments of Formula (Id-1), (Ia-1), (Ib-1), (Ic-1), (Id-2), (Ia-2), (Ib-2), and (Ic-2), R³ and R¹ are taken together with the atoms to which they are attached to form an optionally substituted 5-6-membered heterocyclic ring (e.g., tetrahydrofuran, tetrahydropyran, 2,3-dihydrobenzofuran, or morpholine). In some embodiments, R³ and R¹ combine to form an optionally substituted heterocyclic ring selected from the group consisting of teterahydrofuran, tetrahydropyran, morpholine, dioxane, and 2,3-dihydrobenzofuran.

In some embodiments of Formula (I), (Ia), (Ib), and (Ic), R¹ and R^{1′} are each independently hydrogen or optionally substituted phenyl or pyridyl; R² and R^{2′} are each independently hydrogen; and R³ is hydrogen or optionally substituted —(C₁-C₆)alkyl. In some embodiments, R¹ and R^{1′} are each independently hydrogen or optionally substituted phenyl or pyridyl; R² and R^{2′} are each independently hydrogen; and R³ is hydrogen. In some embodiments, one of R¹ and R^{1′} is hydrogen and the other is optionally substituted phenyl; R² and R^{2′} are each independently hydrogen; and R³ is hydrogen or optionally substituted —(C₁-C₆)alkyl. In some embodiments, one of R¹ and R^{1′} is hydrogen and the other is optionally substituted pyridyl; R² and R^{2′} are each independently hydrogen; and R³ is hydrogen or optionally substituted —(C₁-C₆)alkyl. In some embodiments, R¹ and R^{1′} are each independently hydrogen or optionally substituted phenyl or pyridyl; R² and R^{2′} are each independently hydrogen or optionally substituted —(C₁-C₆)alkyl; and R³ is hydrogen. In some embodiments, one of R¹ and R^{1′} is hydrogen and the other is optionally substituted phenyl; R² and R^{2′} are each independently hydrogen or optionally substituted —(C₁-C₆)alkyl; and R³ is hydrogen. In some embodiments, one of R¹ and R^{1′} is hydrogen and the other is optionally substituted pyridyl; R² and R^{2′} are each independently hydrogen or optionally substituted —(C₁-C₆)alkyl; and R³ is hydrogen.

In some embodiments of Formula (Id-1), (Ia-1), (Ib-1), (Ic-1), (Id-2), (Ia-2), (Ib-2), and (Ic-2), R¹ is optionally substituted phenyl or pyridyl; R² and R^{2′} are each independently hydrogen; and R³ is hydrogen or optionally substituted —(C₁-C₆)alkyl. In some embodiments, R¹ is optionally substituted phenyl or pyridyl; R² and R^{2′} are each independently hydrogen; and R³ is hydrogen. In some embodiments, R¹ is optionally substituted phenyl or pyridyl; R² and R^{2′} are each independently hydrogen or optionally substituted —(C₁-C₆)alkyl; and R³ is hydrogen. In some embodiments, R¹ is optionally substituted phenyl; R² and R^{2′} are each independently hydrogen or optionally substituted —(C₁-C₆)alkyl; and R³ is hydrogen. In some embodiments, R¹ is optionally substituted pyridyl; R² and R^{2′} are each independently hydrogen or optionally substituted —(C₁-C₆)alkyl; and R³ is hydrogen.

In some embodiments of Formula (I), (Ia), (Ib), (Ic), (Id-1), (Ia-1), (Ib-1), (Ic-1), (Id-2), (Ia-2), (Ib-2), and (Ic-2), R⁴ is optionally substituted aryl (e.g., phenyl) or heteroaryl (e.g., pyridyl, benzofuranyl, benzoxazolyl, or benzothiazolyl). In some embodiments, the aryl or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵. In some embodiments, the aryl or heteroaryl is optionally substituted with one or more substituents selected from the group consisting of halogen, oxo, —CN, —R⁵, —OR⁵, —S(O)₂NR⁵R^{5′}, —S(O)NR⁵R^{5′}, and —C(O)R⁵. In some embodiments, aryl or heteroaryl is optionally substituted with one or more substituents selected from the group consisting of —R⁵ and —OR⁵.

In some embodiments, R⁴ is aryl or heteroaryl optionally substituted with one or more —R⁵ or —OR⁵, or two R⁵ on adjacent atoms, together with the atoms to which they are attached, form a heterocycloalkyl ring, optionally substituted with one or more R⁶. In some embodiments, R⁴, including any substitution thereof, is selected from the group consisting of:

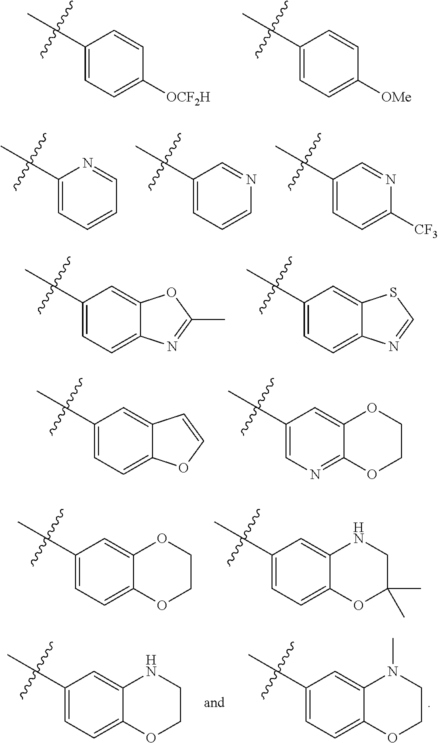

In some embodiments, R⁴ is 6-membered aryl or heteroaryl substituted with two —R⁵, selected from the group consisting of —OR⁶ and —NR⁶R^{6′}, on adjacent atoms of R⁴, that together with the atoms to which they are attached form a heterocycloalkyl ring fused to R⁴ that is optionally substituted with one or more R⁶, selected from the group consisting of —H and —(C₁-C₆)alkyl.

In some embodiments, each R⁵ is independently —H, —(C₁-C₆)alkyl (e.g., methyl, optionally substituted with one or more halogen), halogen, —CN, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, or —C(O)OR⁶. In some embodiments, each R⁵ is independently —H or optionally substituted —(C₁-C₆)alkyl.

In some embodiments, two R⁵ on adjacent atoms, together with the atoms to which they are attached, form an aryl ring optionally substituted with one or more R⁶. In some embodiments, two R⁵ on adjacent atoms, together with the atoms to which they are attached, form a heteroaryl ring optionally substituted with one or more R⁶. In some embodiments, two R⁵ on adjacent atoms together with the atoms to which they are attached form a (C₃-C₈)cycloalkyl ring optionally substituted with one or more R⁶. In some embodiments, two R⁵ on adjacent atoms together with the atoms to which they are attached form a heterocycloalkyl ring optionally substituted with one or more R⁶.

In some embodiments, each R⁶ is independently —H, —(C₁-C₆)alkyl (e.g., methyl), halogen, —CN, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR'S(O)R^{7′}, —C(O)R⁷, or —C(O)OR⁷. In some embodiments, R⁶ is —(C₁-C₆)alkyl.

In some embodiments, Y is a bond.

In some embodiments, Y is —CR⁵R^{5′}—.

In some embodiments, Y is —NR⁵(CR⁵R^{5′})ₜ—.

In some embodiments, Y is —O—.

Nonlimiting examples of the compounds of the disclosure include:

| Example | Structure | Name |
|---|---|---|
| 1 | 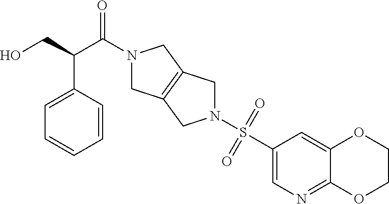 [SMILES: O=C([C@H](CO)c1ccccc1)N1CC2=C(C1)CN(S(=O)(=O)c1cnc3c(c1)OCCO3)C2] | (S)-1-(5-((2,3-dihydro- [1,4]dioxino[2,3-b]pyridin-7- yl)sulfonyl)-3,4,5,6- tetrahydropyrrolo[3,4-c]pyrrol- 2(1H)-yl)-3-hydroxy-2-phenylpropan- 1-one |
| 2 | 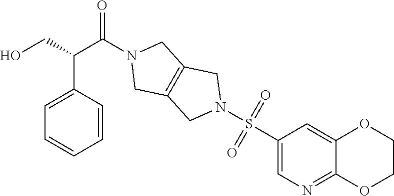 [SMILES: O=C([C@@H](CO)c1ccccc1)N1CC2=C(C1)CN(S(=O)(=O)c1cnc3c(c1)OCCO3)C2] | (R)-1-(5-((2,3-dihydro- [1,4]dioxino[2,3-b]pyridin-7- yl)sulfonyl)-3,4,5,6- tetrahydropyrrolo[3,4-c]pyrrol- 2(1H)-yl)-3-hydroxy-2-phenylpropan- 1-one |
| 3^{a} | 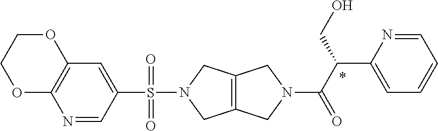 [SMILES: O=C([C@H](CO)c1ccccn1)N1CC2=C(C1)CN(S(=O)(=O)c1cnc3c(c1)OCCO3)C2] | (R or S)-1-(5-((2,3-dihydro- [1,4]dioxino[2,3-b]pyridin-7- yl)sulfonyl)-3,4,5,6- tetrahydropyrrolo[3,4-c]pyrrol- 2(1H)-yl)-3-hydroxy-2-(pyridin-2- yl)propan-1-one |
| 4^{a} | 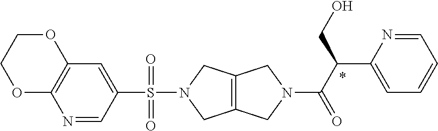 [SMILES: O=C([C@@H](CO)c1ccccn1)N1CC2=C(C1)CN(S(=O)(=O)c1cnc3c(c1)OCCO3)C2] | (S or R)-1-(5-((2,3-dihydro- [1,4]dioxino[2,3-b]pyridin-7- yl)sulfonyl)-3,4,5,6- tetrahydropyrrolo[3,4-c]pyrrol- 2(1H)-yl)-3-hydroxy-2-(pyridin-2- yl)propan-1-one |
| 5^{b} | 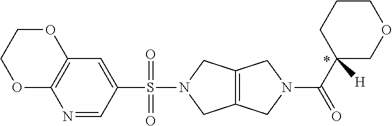 [SMILES: O=C([C@@H]1CCCOC1)N1CC2=C(C1)CN(S(=O)(=O)c1cnc3c(c1)OCCO3)C2] | (R or S)-(5-((2,3-dihydro- [1,4]dioxino[2,3-b]pyridin-7- yl)sulfonyl)-3,4,5,6- tetrahydropyrrolo[3,4-c]pyrrol- 2(1H)-yl)(tetrahydro-2H-pyran-3- yl)methanone |
| 6^{b} | 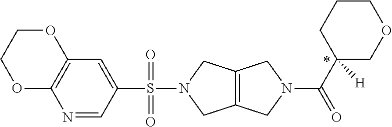 [SMILES: O=C([C@H]1CCCOC1)N1CC2=C(C1)CN(S(=O)(=O)c1cnc3c(c1)OCCO3)C2] | (S or R)-(5-((2,3-dihydro- [1,4]dioxino[2,3-b]pyridin-7- yl)sulfonyl)-3,4,5,6- tetrahydropyrrolo[3,4-c]pyrrol- 2(1H)-yl)(tetrahydro-2H-pyran-3- yl)methanone |
| 7 | 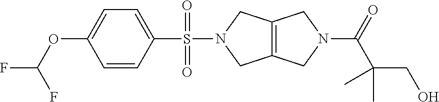 [SMILES: CC(C)(CO)C(=O)N1CC2=C(C1)CN(S(=O)(=O)c1ccc(OC(F)F)cc1)C2] | 1-(5-((4- (difluoromethoxy)phenyl)sulfonyl)- 3,4,5,6-tetrahydropyrrolo[3,4- c]pyrrol-2(1H)-yl)-3-hydroxy-2,2- dimethylpropan-1-one |
| 8 | 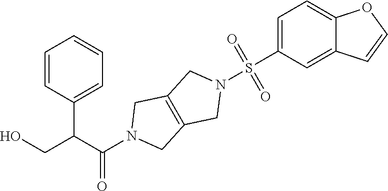 [SMILES: O=C(C(CO)c1ccccc1)N1CC2=C(C1)CN(S(=O)(=O)c1ccc3occc3c1)C2] | 1-(5-(benzofuran-5-ylsulfonyl)- 3,4,5,6-tetrahydropyrrolo[3,4- c]pyrrol-2(1H)-yl)-3-hydroxy-2- phenylpropan-1-one |
| 9 | 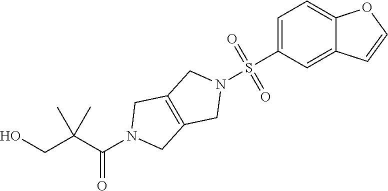 [SMILES: CC(C)(CO)C(=O)N1CC2=C(C1)CN(S(=O)(=O)c1ccc3occc3c1)C2] | 1-(5-(benzofuran-5-ylsulfonyl)- 3,4,5,6-tetrahydropyrrolo[3,4- c]pyrrol-2(1H)-yl)-3-hydroxy-2,2- dimethylpropan-1-one |
| 10 | 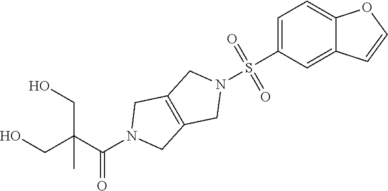 [SMILES: CC(CO)(CO)C(=O)N1CC2=C(C1)CN(S(=O)(=O)c1ccc3occc3c1)C2] | 1-(5-(benzofuran-5-ylsulfonyl)- 3,4,5,6-tetrahydropyrrolo[3,4- c]pyrrol-2(1H)-yl)-3-hydroxy-2- (hydroxymethyl)-2-methylpropan-1- one |
| 11 | 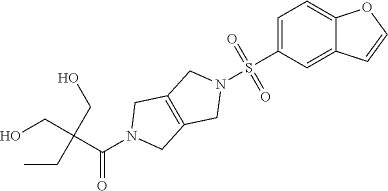 [SMILES: CCC(CO)(CO)C(=O)N1CC2=C(C1)CN(S(=O)(=O)c1ccc3occc3c1)C2] | 1-(5-(benzofuran-5-ylsulfonyl)- 3,4,5,6-tetrahydropyrrolo[3,4- c]pyrrol-2(1H)-yl)-2,2- bis(hydroxymethyl)butan-1-one |
| 12 | 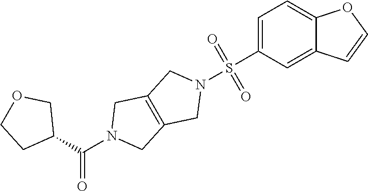 [SMILES: O=C([C@@H]1CCOC1)N1CC2=C(C1)CN(S(=O)(=O)c1ccc3occc3c1)C2] | (R)-(5-(benzofuran-5-ylsulfonyl)- 3,4,5,6-tetrahydropyrrolo[3,4- c]pyrrol-2(1H)-yl)(tetrahydrofuran-3- yl)methanone |
| 13 | 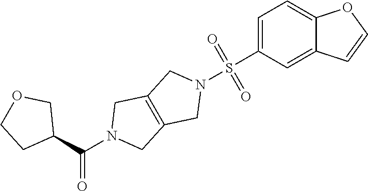 [SMILES: O=C([C@H]1CCOC1)N1CC2=C(C1)CN(S(=O)(=O)c1ccc3occc3c1)C2] | (S)-(5-(benzofuran-5-ylsulfonyl)- 3,4,5,6-tetrahydropyrrolo[3,4- c]pyrrol-2(1H)-yl)(tetrahydrofuran-3- yl)methanone |
| 14 | 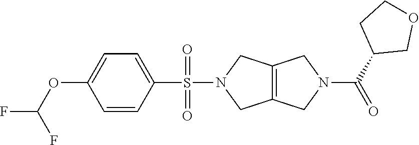 [SMILES: O=C([C@@H]1CCOC1)N1CC2=C(C1)CN(S(=O)(=O)c1ccc(OC(F)F)cc1)C2] | (R)-(5-((4- (difluoromethoxy)phenyl)sulfonyl)- 3,4,5,6-tetrahydropyrrolo[3,4- c]pyrrol-2(1H)-yl)(tetrahydrofuran-3- yl)methanone |
| 15 | 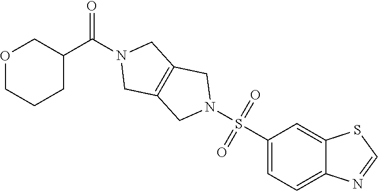 [SMILES: O=C(C1CCCOC1)N1CC2=C(C1)CN(S(=O)(=O)c1ccc3ncsc3c1)C2] | (5-(benzo[d]thiazol-6-ylsulfonyl)- 3,4,5,6-tetrahydropyrrolo[3,4- c]pyrrol-2(1H)-yl)(tetrahydro-2H- pyran-3-yl)methanone |
| 16 | 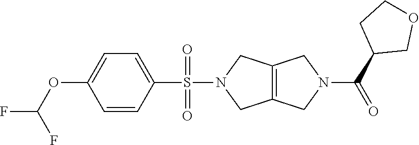 [SMILES: O=C([C@H]1CCOC1)N1CC2=C(C1)CN(S(=O)(=O)c1ccc(OC(F)F)cc1)C2] | (S)-(5-((4- (difluoromethoxy)phenyl)sulfonyl)- 3,4,5,6-tetrahydropyrrolo[3,4- c]pyrrol-2(1H)-yl)(tetrahydrofuran-3- yl)methanone |
| 17 | 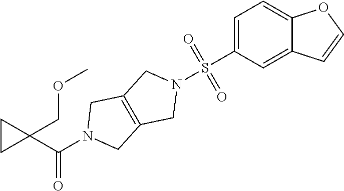 [SMILES: COCC1(C(=O)N2CC3=C(C2)CN(S(=O)(=O)c2ccc4occc4c2)C3)CC1] | (5-(benzofuran-5-ylsulfonyl)-3,4,5,6- tetrahydropyrrolo[3,4-c]pyrrol- 2(1H)-yl)(1- (methoxymethyl)cyclopropyl) methanone |
| 18 | 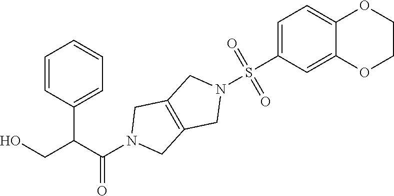 [SMILES: O=C(C(CO)c1ccccc1)N1CC2=C(C1)CN(S(=O)(=O)c1ccc3c(c1)OCCO3)C2] | 1-(5-((2,3- dihydrobenzo[b][1,4]dioxin-6- yl)sulfonyl)-3,4,5,6- tetrahydropyrrolo[3,4-c]pyrrol- 2(1H)-yl)-3-hydroxy-2-phenylpropan- 1-one |
| 19 | 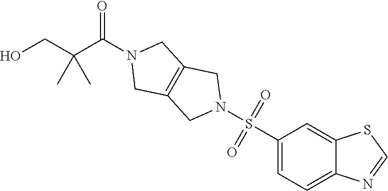 [SMILES: CC(C)(CO)C(=O)N1CC2=C(C1)CN(S(=O)(=O)c1ccc3ncsc3c1)C2] | 1-(5-(benzo[d]thiazol-6-ylsulfonyl)- 3,4,5,6-tetrahydropyrrolo[3,4- c]pyrrol-2(1H)-yl)-3-hydroxy-2,2- dimethylpropan-1-one |
| 20 | 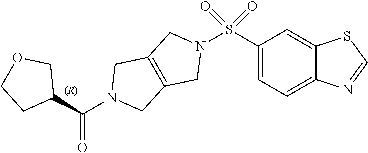 | (R)-(5-(benzo[d]thiazol-6-ylsulfonyl)- 3,4,5,6-tetrahydropyrrolo[3,4- c]pyrrol-2(1H)-yl)(tetrahydrofuran-3- yl)methanone |
| 21 | 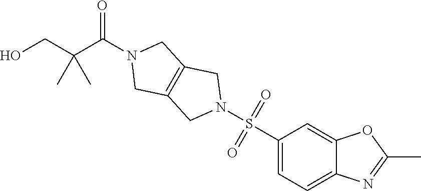 [SMILES: Cc1nc2ccc(S(=O)(=O)N3CC4=C(CN(C(=O)C(C)(C)CO)C4)C3)cc2o1] | 3-hydroxy-2,2-dimethyl-1-(5-((2- methylbenzo[d]oxazol-6-yl)sulfonyl)- 3,4,5,6-tetrahydropyrrolo[3,4- c]pyrrol-2(1H)-yl)propan-1-one |
| 22 | 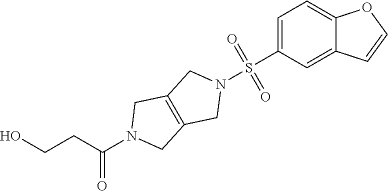 [SMILES: O=C(CCO)N1CC2=C(C1)CN(S(=O)(=O)c1ccc3occc3c1)C2] | 1-(5-(benzofuran-5-ylsulfonyl)- 3,4,5,6-tetrahydropyrrolo[3,4- c]pyrrol-2(1H)-yl)-3-hydroxypropan- 1-one |
| 23 | 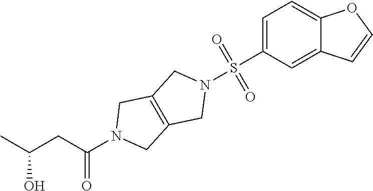 [SMILES: C[C@@H](O)CC(=O)N1CC2=C(C1)CN(S(=O)(=O)c1ccc3occc3c1)C2] | (R)-1-(5-(benzofuran-5-ylsulfonyl)- 3,4,5,6-tetrahydropyrrolo[3,4- c]pyrrol-2(1H)-yl)-3-hydroxybutan-1- one |
| 24 | 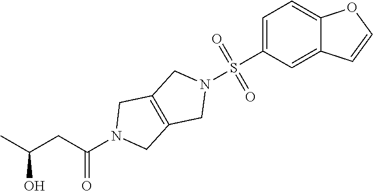 [SMILES: C[C@H](O)CC(=O)N1CC2=C(C1)CN(S(=O)(=O)c1ccc3occc3c1)C2] | (S)-1-(5-(benzofuran-5-ylsulfonyl)- 3,4,5,6-tetrahydropyrrolo[3,4- c]pyrrol-2(1H)-yl)-3-hydroxybutan-1- one |
| 25 | 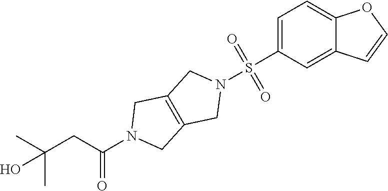 [SMILES: CC(C)(O)CC(=O)N1CC2=C(C1)CN(S(=O)(=O)c1ccc3occc3c1)C2] | 1-(5-(benzofuran-5-ylsulfonyl)- 3,4,5,6-tetrahydropyrrolo[3,4- c]pyrrol-2(1H)-yl)-3-hydroxy-3- methylbutan-1-one |
| 26 | 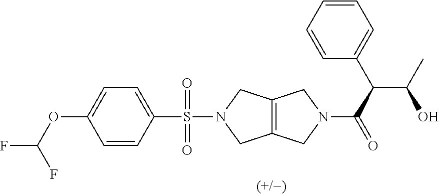 [SMILES: C[C@@H](O)[C@@H](C(=O)N1CC2=C(C1)CN(S(=O)(=O)c1ccc(OC(F)F)cc1)C2)c1ccccc1] | (2S,3R and 2R,3S)-1-(5-((4- (difluoromethoxy)phenyl)sulfonyl)- 3,4,5,6-tetrahydropyrrolo[3,4- c]pyrrol-2(1H)-yl)-3-hydroxy-2- phenylbutan-1-one |
| 27 | 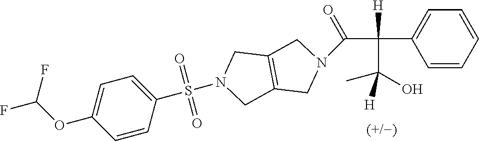 [SMILES: C[C@@H](O)[C@H](C(=O)N1CC2=C(C1)CN(S(=O)(=O)c1ccc(OC(F)F)cc1)C2)c1ccccc1] | (2R,3R and 2S,3S)-1-(5-((4- (difluoromethoxy)phenyl)sulfonyl)- 3,4,5,6-tetrahydropyrrolo[3,4- c]pyrrol-2(1H)-yl)-3-hydroxy-2- phenylbutan-1-one |
| 28 | 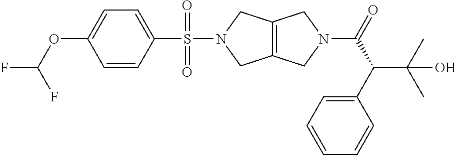 [SMILES: CC(C)(O)[C@@H](C(=O)N1CC2=C(C1)CN(S(=O)(=O)c1ccc(OC(F)F)cc1)C2)c1ccccc1] | (S)-1-(5-((4- (difluoromethoxy)phenyl)sulfonyl)- 3,4,5,6-tetrahydropyrrolo[3,4- c]pyrrol-2(1H)-yl)-3-hydroxy-3- methyl-2-phenylbutan-1-one |
| 29 | 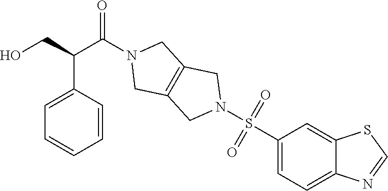 [SMILES: O=C([C@H](CO)c1ccccc1)N1CC2=C(C1)CN(S(=O)(=O)c1ccc3ncsc3c1)C2] | (S)-1-(5-(benzo[d]thiazol-6- ylsulfonyl)-3,4,5,6- tetrahydropyrrolo[3,4-c]pyrrol- 2(1H)-yl)-3-hydroxy-2-phenylpropan- 1-one |
| 30 | 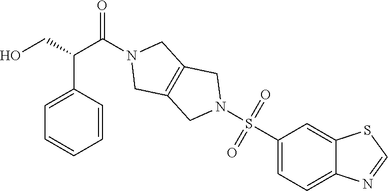 [SMILES: O=C([C@@H](CO)c1ccccc1)N1CC2=C(C1)CN(S(=O)(=O)c1ccc3ncsc3c1)C2] | (R)-1-(5-(benzo[d]thiazol-6- ylsulfonyl)-3,4,5,6- tetrahydropyrrolo[3,4-c]pyrrol- 2(1H)-yl)-3-hydroxy-2-phenylpropan- 1-one |
| 31 | 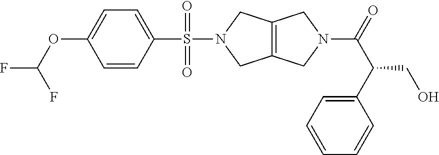 [SMILES: O=C([C@H](CO)c1ccccc1)N1CC2=C(C1)CN(S(=O)(=O)c1ccc(OC(F)F)cc1)C2] | (S)-1-(5-((4- (difluoromethoxy)phenyl)sulfonyl)- 3,4,5,6-tetrahydropyrrolo[3,4- c]pyrrol-2(1H)-yl)-3-hydroxy-2- phenylpropan-1-one |
| 32 | 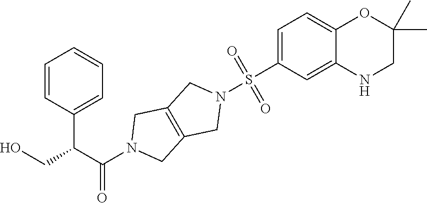 [SMILES: CC1(C)CNc2cc(S(=O)(=O)N3CC4=C(CN(C(=O)[C@H](CO)c5ccccc5)C4)C3)ccc2O1] | (S)-1-(5-((2,2-dimethyl-3,4-dihydro- 2H-benzo[b][1,4]oxazin-6- yl)sulfonyl)-3,4,5,6- tetrahydropyrrolo[3,4-c]pyrrol- 2(1H)-yl)-3-hydroxy-2-phenylpropan- 1-one |
| 33^{c} | 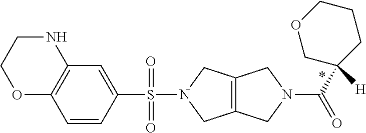 [SMILES: O=C([C@H]1CCCOC1)N1CC2=C(C1)CN(S(=O)(=O)c1ccc3c(c1)NCCO3)C2] | (S or R)-(5-((3,4-dihydro-2H- benzo[b][1,4]oxazin-6-yl)sulfonyl)- 3,4,5,6-tetrahydropyrrolo[3,4- c]pyrrol-2(1H)-yl)(tetrahydro-2H- pyran-3-yl)methanone |
| 34^{c} | 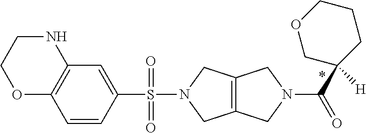 [SMILES: O=C([C@@H]1CCCOC1)N1CC2=C(C1)CN(S(=O)(=O)c1ccc3c(c1)NCCO3)C2] | (R or S)-(5-((3,4-dihydro-2H- benzo[b][1,4]oxazin-6-yl)sulfonyl)- 3,4,5,6-tetrahydropyrrolo[3,4- c]pyrrol-2(1H)-yl)(tetrahydro-2H- pyran-3-yl)methanone |
| 35 | 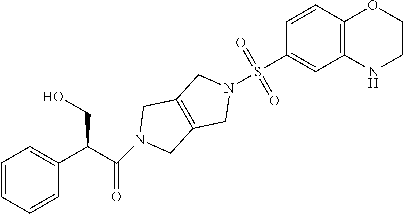 [SMILES: O=C([C@H](CO)c1ccccc1)N1CC2=C(C1)CN(S(=O)(=O)c1ccc3c(c1)NCCO3)C2] | (S)-1-(5-((3,4-dihydro-2H- benzo[b][1,4]oxazin-6-yl)sulfonyl)- 3,4,5,6-tetrahydropyrrolo[3,4- c]pyrrol-2(1H)-yl)-3-hydroxy-2- phenylpropan-1-one |
| 36 | 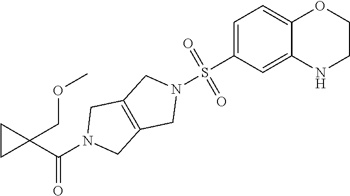 [SMILES: COCC1(C(=O)N2CC3=C(C2)CN(S(=O)(=O)c2ccc4c(c2)NCCO4)C3)CC1] | (5-((3,4-dihydro-2H- benzo[b][1,4]oxazin-6-yl)sulfonyl)- 3,4,5,6-tetrahydropyrrolo[3,4- c]pyrrol-2(1H)-yl)(1- (methoxymethyl)cyclopropyl) methanone |
| 37 | 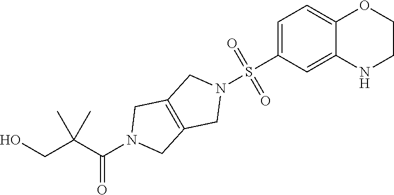 [SMILES: CC(C)(CO)C(=O)N1CC2=C(C1)CN(S(=O)(=O)c1ccc3c(c1)NCCO3)C2] | 1-(5-((3,4-dihydro-2H- benzo[b][1,4]oxazin-6-yl)sulfonyl)- 3,4,5,6-tetrahydropyrrolo[3,4- c]pyrrol-2(1H)-yl)-3-hydroxy-2,2- dimethylpropan-1-one |
| 38 | 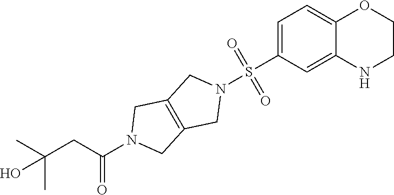 [SMILES: CC(C)(O)CC(=O)N1CC2=C(C1)CN(S(=O)(=O)c1ccc3c(c1)NCCO3)C2] | 1-(5-((3,4-dihydro-2H- benzo[b][1,4]oxazin-6-yl)sulfonyl)- 3,4,5,6-tetrahydropyrrolo[3,4- c]pyrrol-2(1H)-yl)-3-hydroxy-3- methylbutan-1-one |
| 39 | 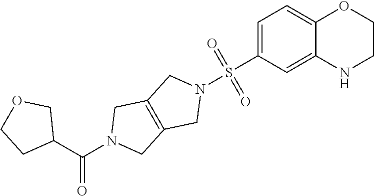 [SMILES: O=C(C1CCOC1)N1CC2=C(C1)CN(S(=O)(=O)c1ccc3c(c1)NCCO3)C2] | (5-((3,4-dihydro-2H- benzo[b][1,4]oxazin-6-yl)sulfonyl)- 3,4,5,6-tetrahydropyrrolo[3,4- c]pyrrol-2(1H)-yl)(tetrahydrofuran-3- yl)methanone |
| 40 | 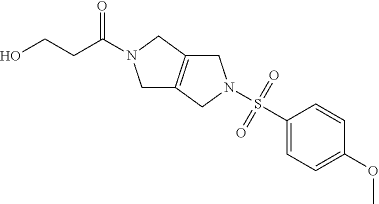 [SMILES: COc1ccc(S(=O)(=O)N2CC3=C(CN(C(=O)CCO)C3)C2)cc1] | 3-hydroxy-1-(5-((4- methoxyphenyl)sulfonyl)-3,4,5,6- tetrahydropyrrolo[3,4-c]pyrrol- 2(1H)-yl)propan-1-one |
| 41^{d} | 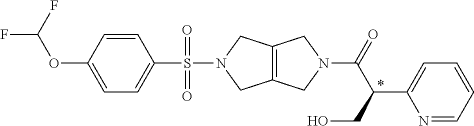 [SMILES: O=C([C@H](CO)c1ccccn1)N1CC2=C(C1)CN(S(=O)(=O)c1ccc(OC(F)F)cc1)C2] | (S or R)-1-(5-((4- (difluoromethoxy)phenyl)sulfonyl)- 3,4,5,6-tetrahydropyrrolo[3,4- c]pyrrol-2(1H)-yl)-3-hydroxy-2- (pyridin-2-yl)propan-1-one |
| 42^{d} | 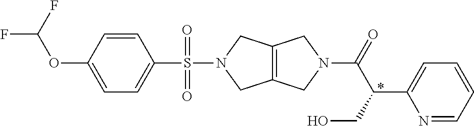 [SMILES: O=C([C@@H](CO)c1ccccn1)N1CC2=C(C1)CN(S(=O)(=O)c1ccc(OC(F)F)cc1)C2] | (R or S)-1-(5-((4- (difluoromethoxy)phenyl)sulfonyl)- 3,4,5,6-tetrahydropyrrolo[3,4- c]pyrrol-2(1H)-yl)-3-hydroxy-2- (pyridin-2-yl)propan-1-one |
| 43 | 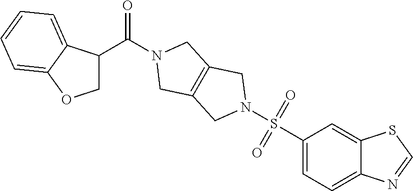 [SMILES: O=C(C1COc2ccccc21)N1CC2=C(C1)CN(S(=O)(=O)c1ccc3ncsc3c1)C2] | (5-(benzo[d]thiazol-6-ylsulfonyl)- 3,4,5,6-tetrahydropyrrolo[3,4- c]pyrrol-2(1H)-yl)(2,3- dihydrobenzofuran-3-yl)methanone |
| 44^{e} | 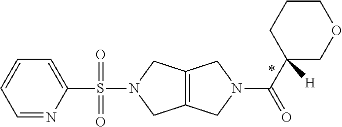 [SMILES: O=C([C@@H]1CCCOC1)N1CC2=C(C1)CN(S(=O)(=O)c1ccccn1)C2] | (R or S)-(5-(pyridin-2-ylsulfonyl)- 3,4,5,6-tetrahydropyrrolo[3,4- c]pyrrol-2(1H)-yl)(tetrahydro-2H- pyran-3-yl)methanone |
| 45^{e} | 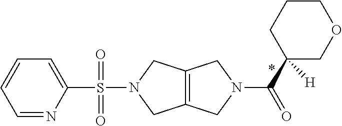 [SMILES: O=C([C@H]1CCCOC1)N1CC2=C(C1)CN(S(=O)(=O)c1ccccn1)C2] | (S or R)-(5-(pyridin-2-ylsulfonyl)- 3,4,5,6-tetrahydropyrrolo[3,4- c]pyrrol-2(1H)-yl)(tetrahydro-2H- pyran-3-yl)methanone |
| 46 | 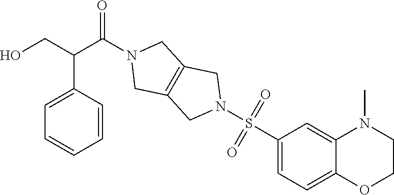 [SMILES: CN1CCOc2ccc(S(=O)(=O)N3CC4=C(CN(C(=O)C(CO)c5ccccc5)C4)C3)cc21] | 3-hydroxy-1-(5-((4-methyl-3,4- dihydro-2H-benzo[b][1,4]oxazin-6- yl)sulfonyl)-3,4,5,6- tetrahydropyrrolo[3,4-c]pyrrol- 2(1H)-yl)-2-phenylpropan-1-one |
| 47 | 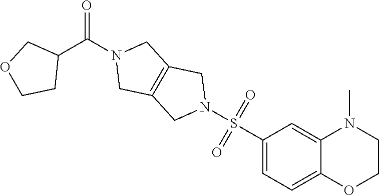 [SMILES: CN1CCOc2ccc(S(=O)(=O)N3CC4=C(CN(C(=O)C5CCOC5)C4)C3)cc21] | (5-((4-methyl-3,4-dihydro-2H- benzo[b][1,4]oxazin-6-yl)sulfonyl)- 3,4,5,6-tetrahydropyrrolo[3,4- c]pyrrol-2(1H)-yl)(tetrahydrofuran-3- yl)methanone |
| 48 | 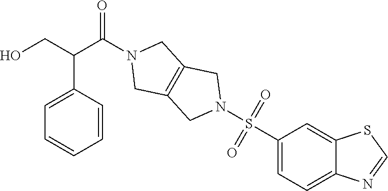 [SMILES: O=C(C(CO)c1ccccc1)N1CC2=C(C1)CN(S(=O)(=O)c1ccc3ncsc3c1)C2] | 1-(5-(benzo[d]thiazol-6-ylsulfonyl)- 3,4,5,6-tetrahydropyrrolo[3,4- c]pyrrol-2(1H)-yl)-3-hydroxy-2- phenylpropan-1-one |
| 49 | 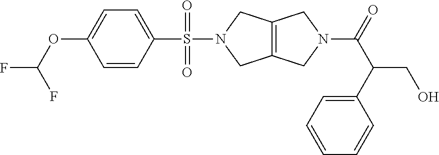 [SMILES: O=C(C(CO)c1ccccc1)N1CC2=C(C1)CN(S(=O)(=O)c1ccc(OC(F)F)cc1)C2] | 1-(5-((4- (difluoromethoxy)phenyl)sulfonyl)- 3,4,5,6-tetrahydropyrrolo[3,4- c]pyrrol-2(1H)-yl)-3-hydroxy-2- phenylpropan-1-one |
| 50 | 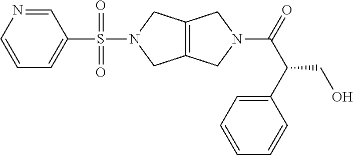 [SMILES: O=C([C@H](CO)c1ccccc1)N1CC2=C(C1)CN(S(=O)(=O)c1cccnc1)C2] | (2S)-3-hydroxy-2-phenyl-1-[5- (pyridine-3-sulfonyl)- 1H,2H,3H,4H,5H,6H-pyrrolo[3,4- c]pyrrol-2-yl]propan-1-one |
| 51 | 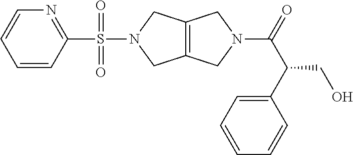 [SMILES: O=C([C@H](CO)c1ccccc1)N1CC2=C(C1)CN(S(=O)(=O)c1ccccn1)C2] | (2S)-3-hydroxy-2-phenyl-1-[5- (pyridine-2-sulfonyl)- 1H,2H,3H,4H,5H,6H-pyrrolo[3,4- c]pyrrol-2-yl]propan-1-one |
| 52 | 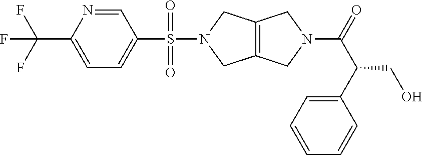 [SMILES: O=C([C@H](CO)c1ccccc1)N1CC2=C(C1)CN(S(=O)(=O)c1ccc(C(F)(F)F)nc1)C2] | (2S)-3-hydroxy-2-phenyl-1-(5-{[6- (trifluoromethyl)pyridin-3- yl]sulfonyl}-1H,2H,3H,4H,5H,6H- pyrrolo[3,4-c]pyrrol-2-yl)propan-1- one |
| 53 | 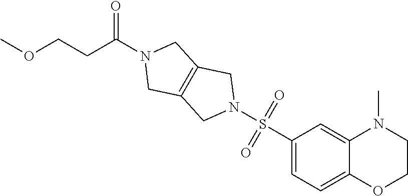 [SMILES: COCCC(=O)N1CC2=C(C1)CN(S(=O)(=O)c1ccc3c(c1)N(C)CCO3)C2] | 3-methoxy-1-(5-((4-methyl-3,4- dihydro-2H-benzo[b][1,4]oxazin-6- yl)sulfonyl)-3,4,5,6- tetrahydropyrrolo[3,4-c]pyrrol- 2(1H)-yl)propan-1-one |
| 54 | 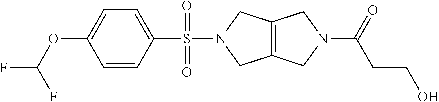 [SMILES: O=C(CCO)N1CC2=C(C1)CN(S(=O)(=O)c1ccc(OC(F)F)cc1)C2] | 1-(5-((4- (difluoromethoxy)phenyl)sulfonyl)- 3,4,5,6-tetrahydropyrrolo[3,4- c]pyrrol-2(1H)-yl)-3-hydroxypropan- 1-one |
| 55 | 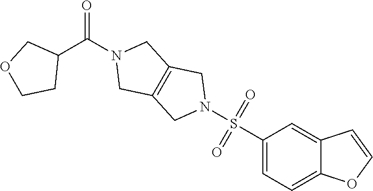 [SMILES: O=C(C1CCOC1)N1CC2=C(C1)CN(S(=O)(=O)c1ccc3occc3c1)C2] | (5-(benzofuran-5-ylsulfonyl)-3,4,5,6- tetrahydropyrrolo[3,4-c]pyrrol- 2(1H)-yl)(tetrahydrofuran-3- yl)methanone |
| 56 | 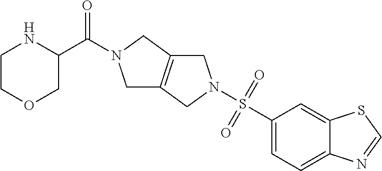 [SMILES: O=C(C1COCCN1)N1CC2=C(C1)CN(S(=O)(=O)c1ccc3ncsc3c1)C2] | (5-(benzo[d]thiazol-6-ylsulfonyl)- 3,4,5,6-tetrahydropyrrolo[3,4- c]pyrrol-2(1H)-yl)(morpholin-3- yl)methanone |
| 57 | 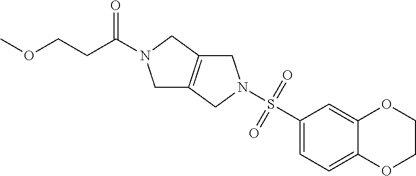 [SMILES: COCCC(=O)N1CC2=C(C1)CN(S(=O)(=O)c1ccc3c(c1)OCCO3)C2] | 1-(5-((2,3- dihydrobenzo[b][1,4]dioxin-6- yl)sulfonyl)-3,4,5,6- tetrahydropyrrolo[3,4-c]pyrrol- 2(1H)-yl)-3-methoxypropan-1-one |
| 58 | 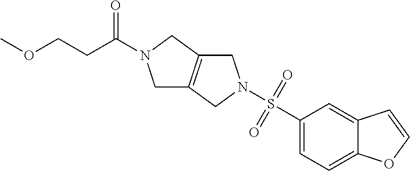 [SMILES: COCCC(=O)N1CC2=C(C1)CN(S(=O)(=O)c1ccc3occc3c1)C2] | 1-(5-(benzofuran-5-ylsulfonyl)- 3,4,5,6-tetrahydropyrrolo[3,4- c]pyrrol-2(1H)-yl)-3-methoxypropan- 1-one |

^{a}Compounds 3 and 4 are enantiomers, but absolute stereochemistry is undetermined (*);
^{b}Compounds 5 and 6 are enantiomers, but absolute stereochemistry is undetermined (*);
^{c}Compounds 33 and 34 are enantiomers, but absolute stereochemistry is undetermined (*);
^{d}Compounds 41 and 42 are enantiomers, but absolute stereochemistry is undetermined (*);
^{e}Compounds 44 and 45 are enantiomers, but absolute stereochemistry is undetermined (*).

### Method of Synthesizing the Compounds

The compounds of the present disclosure may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of Formula (I).

Those skilled in the art will recognize if a stereocenter exists in the compounds of Formula (I). When a compound is desired as a single enantiomer or diastereomer, it may be obtained by stereospecific synthesis or by resolution of the final product or of any convenient intermediate. For example, enantiomerically pure compounds of Formula (I) can be prepared using enantiomerically pure chiral building blocks. Alternatively, racemic mixtures of the final compounds or a racemic mixture of an advanced intermediate can be subjected to chiral purification as described herein below to deliver the desired enantiomerically pure intermediates or final compounds. In the instances where an advanced intermediate is purified into its individual enantiomers, each individual enantiomer can be carried on separately to deliver the final enantiomerically pure compounds of Formula (I). Resolution of the final product, an intermediate, or a starting material may be effected by any suitable method known in the art. See, for example, “Stereochemistry of Organic Compounds” by E. L. Eliel, S. H. Wilen, and L. N. Mander (Wiley-Interscience, 1994). The absolute stereochemistry of compounds obtained by chiral resolution or chiral purification may or may not be determined. Enantiomerically pure compounds with undetermined absolute stereochemistry have been drawn as a single enantiomer chosen arbitrarily and are marked with an asterisk (*) at the chiral carbon herein.

The compounds described herein may be made from commercially available starting materials or synthesized using known organic, inorganic, and/or enzymatic processes.

### Preparation of Compounds

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereof as appreciated by those skilled in the art. Preferred methods include but are not limited to those methods described in the synthetic examples below.

It should be understood that in the description and formula shown above, the various groups Y, R¹, R^{1′}, R², R^{2′}, R³, R⁴, R⁸, R^{8′}, R⁹, R^{9′}, R¹⁰, R^{10′}, R¹¹, R^{11′} and other variables are as defined above, except where otherwise indicated.

### Methods of Identifying and Characterizing PKR Activating Compounds

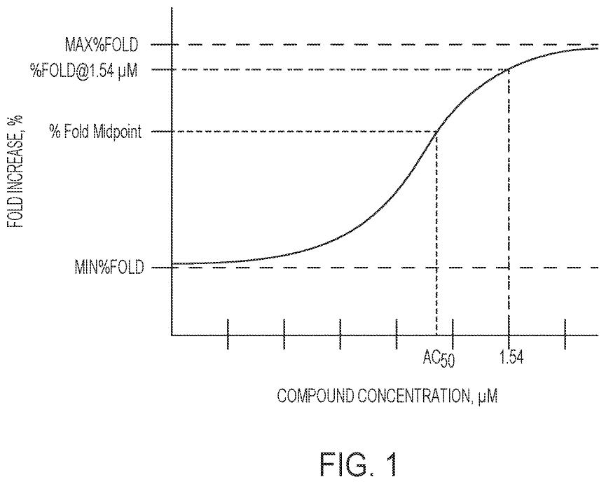

In certain embodiments, specific PKR Activating Compounds (including compounds of Formula (I), as well as additional examples of such compounds) can be identified using the Luminescence Assay Protocol described in Example 47. The PKR Activating Compounds can be selected by obtaining and analyzing data from a dose-response curve for a compound in accordance with the Luminescence Assay Protocol. FIG. 1 shows an exemplary dose-response curve for compounds disclosed herein. The values of AC₅₀ and MAX % Fold are independent of each other (i.e., the value of one does not affect the other). In some embodiments, PKR Activating Compounds can be selected based on the % Fold increase at a given concentration of compound (e.g., 1.54 μM) in the Luminescence Assay Protocol. The % Fold increase at a given concentration is a value which will be impacted by both the potency (AC₅₀) and activity (MAX % Fold).

In some embodiments, the PKR Activating Compound can be selected as a compound of Formula (I) having a % Fold value at 1.54 μM concentration of compound (% Fold@1.54 μM) of at least 75% (e.g., 75%-500%, 75%-250%, or 250%-500%) in an assay (e.g., the Luminescence Assay of Example 47) using a PKR enzyme (e.g., wild type PKR enzyme, or a clinically relevant mutant PKR, such as PKR G332S or PKR R510Q).

In some embodiments, PKR Activating Compounds have a % Fold@1.54 μM of at least 75% (e.g., 75%-500% or 250%-500%) obtained using the Luminescence Assay Protocol of Example 47. PKR Activating Compounds can be identified in accordance with Example 47 by a method comprising the steps of (a) incubating a mixture of phosphoenolpyruvic acid (PEP) and PKR enzyme (e.g., wild type PKR or a clinically relevant PKR mutant enzyme) with a test compound at a concentration of 1.54 μM; (b) adding adenosine-5′-diphosphate (ADP) and a kinase luminescence reporter composition (e.g., Kinase Glo Plus) to the mixture in step (a) under conditions effective to induce luminescence in the presence of a test compound that is a PKR Activating Compound; (c) measuring the luminescence values of the mixture obtained in step (b); (d) determining the % Fold@1.54 μM value for the test compound; and (e) identifying the test compound as a PKR Activating Compound when the test compound has a % Fold@1.54 μM value of at least 75% (e.g., 75-500%, or 250-500%).

### Methods of Using the Disclosed Compounds

In another aspect, the present disclosure relates to a method of activating PKR, including methods of treating a disease or disorder in a patient by administering a therapeutically effective amount of a PKR Activating Compound disclosed herein. For example, the method can comprise administering to a patient in need thereof a therapeutically effective amount of a compound of Formula (I). In some embodiments, the disease or disorder is selected from the group consisting of PKD, SCD (e.g., sickle cell anemia), and thalassemia (e.g., beta-thalassemia). A method of treating a patient diagnosed with a disease, selected from the group consisting of PKD, SCD, and thalassemia, comprises administering a therapeutically effective amount of a compound disclosed herein, including a therapeutically effective amount of a PKR Activating Compound of Formula (I). A method of treating PKD comprises administering a therapeutically effective amount of a compound disclosed herein, including a PKR Activating Compound of Formula (I). A method of treating SCD comprises administering a therapeutically effective amount of a compound disclosed herein, including a PKR Activating Compound of Formula (I). A method of treating thalassemia comprises administering a therapeutically effective amount of a compound disclosed herein, including a PKR Activating Compound of Formula (I).

In other embodiments, the method comprises administering a therapeutically effective amount of a compound of Formula (I) for the treatment of a patient diagnosed with a condition selected from the group consisting of: hereditary non-spherocytic hemolytic anemia, hemolytic anemia (e.g., chronic hemolytic anemia caused by phosphoglycerate kinase deficiency), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), or anemia of chronic diseases. In some embodiments, the disease or disorder is hereditary non-sperocytic hemolytic anemia. In some embodiments, the disease or disorder is SCD (e.g., sickle cell anemia) or thalassemia (e.g., beta-thalassemia). In some embodiments, the disease or disorder is hemolytic anemia (e.g., in a patient diagnosed with PKD). In some embodiments, the disease or disorder is beta thalassemia. In some embodiments, the disease or disorder is SCD.

Another aspect of the disclosure relates to of the use of a PKR Activating Compound for treating a disease or disorder associated with modulation of PKR and/or PKM2. The present disclosure also relates to the use of an activator of PKR and/or PKM2 for the preparation of a medicament used in the treatment of a disease or condition, wherein the medicament comprises a compound of Formula (I). In other embodiments, the present disclosure relates to the use of an activator of PKR and/or PKM2 for the preparation of a medicament used in the treatment of a disease or condition mediated by PKR and/or PKM2, wherein the medicament comprises a compound of Formula (I). The method can comprise administering to a patient in need of a treatment for diseases or disorders associated with modulation of PKR and/or PKM2 an effective amount of the compositions and/or compounds of Formula (I). The method can comprise the use of a PKR Activating Compound and/or a compound of Formula (I) in the preparation of a medicament for the treatment of diseases or disorders associated with modulation (e.g., activation) of PKR and/or PKM2.

In another aspect, the present disclosure is directed to of the use of a PKR Activating Compound treating a disease or disorder associated with activation of PKR and/or PKM2. The use can comprise administering to a patient in need of a treatment for diseases or disorders associated with modulation of PKR and/or PKM2 an effective amount of the compositions and/or compounds of Formula (I). In some embodiments, the disease or disorder is selected from the group consisting of SCD, sickle cell anemia, thalassemia (e.g., beta-thalassemia), hereditary non-spherocytic hemolytic anemia, hemolytic anemia (e.g., chronic hemolytic anemia caused by phosphoglycerate kinase deficiency), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), or anemia of chronic diseases.

In another aspect, the present disclosure is directed to a method of activating PKR and/or PKM2. The method involves administering to a patient in need thereof an effective amount of a compound of Formula (I).

In another aspect, the present disclosure is directed to a method of increasing the lifetime of red blood cells in a patient or ex vivo using an effective amount of a PKR Activating Compound, such as a compound of Formula (I), or to the use of a PKR Activating Compound, such as a compound of Formula (I), in the preparation of a medicament or a composition (e.g., reagent) for increasing the lifetime of red blood cells in a patient or ex vivo using an effective amount of a PKR Activating Compound, such as the compound Formula (I).

In another aspect, the present disclosure is directed to a method of regulating 2,3-diphosphoglycerate levels in blood in a patient or ex vivo using an effective amount of a PKR Activating Compound, such as a compound Formula (I), or to the use of a PKR Activating Compound, such as a compound Formula (I), in the preparation of a medicament or a composition (e.g., reagent) for regulating 2,3-diphosphoglycerate levels in blood in a patient or ex vivo.

In another aspect, the present disclosure is directed to a method of regulating ATP levels in blood in a patient or ex vivo using an effective amount of a PKR Activating Compound, such as a compound Formula (I), or to the use of a PKR Activating Compound, such as a compound Formula (I), in the preparation of a medicament or a composition (e.g., reagent) for regulating ATP levels in blood in a patient or ex vivo.

In another aspect, the present disclosure relates to a method of treating a disease or disorder associated with decreased activity of PKR and/or PKM2 in a subject in need thereof, the method comprising administering to a patient in need thereof an effective amount of a compound of Formula (I). In some embodiments, the disease or disorder is selected from the group consisting of PKD, SCD, sickle cell anemia, thalassemia (e.g., beta-thalassemia), hereditary non-spherocytic hemolytic anemia, hemolytic anemia (e.g., chronic hemolytic anemia caused by phosphoglycerate kinase deficiency), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), or anemia of chronic diseases.

In another embodiment, the present disclosure relates to a compound of Formula (I) or a pharmaceutical composition comprising a compound of the present disclosure and a pharmaceutically acceptable carrier used for the treatment of SCD, sickle cell anemia, thalassemia (e.g., beta-thalassemia), hereditary non-spherocytic hemolytic anemia, hemolytic anemia (e.g., chronic hemolytic anemia caused by phosphoglycerate kinase deficiency), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), or anemia of chronic diseases.

Another aspect of the disclosure is directed to pharmaceutical compositions comprising a compound of Formula (I) and a pharmaceutically acceptable carrier.

In another aspect, the present disclosure relates to a method of treating cancer. The method comprises administering to a patient in need of a treatment for cancer an effective amount of a compound of Formula (I), or a pharmaceutically acceptable salt thereof.

In another aspect, the present disclosure relates to a method for the manufacture of a medicament for treating a disease or condition mediated by PKR and/or PKM2, wherein the medicament comprises a compound of Formula (I). Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The pharmaceutical acceptable carrier may further include an excipient, diluent, or surfactant. Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener, f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection. Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds. The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564 which is hereby incorporated by reference in its entirety. The disclosed compounds can be also formulated as a suppository that can be prepared from fatty emulsions or suspensions; using polyalkylene glycols such as propylene glycol, as the carrier.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

The compounds of the present disclosure can be administered in effective amounts to treat a disease or disorder in subjects. The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

The following numbered embodiments, while non-limiting, are exemplary of certain aspects of the present disclosure:
1. A compound of the Formula I:

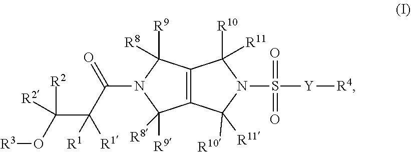

or a pharmaceutically acceptable salt thereof,
wherein:
Y is a bond, —(CR⁵R^{5′})ₜ—, —NR⁵(CR⁵R^{5′})ₜ—, or —O—;
   each R¹, R^{1′}, R², and R^{2′} is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   or R¹ and R⁷, or R² and R^{2′}, together with the atom to which they are attached, can combine to form —(C₃-C₈)cycloalkyl ring, heterocycle, (C₅-C₈)spirocycle or 5- to 8-membered spiroheterocycle;
   or R¹ and R², together with the atoms to which they are attached, can combine to form a —(C₃-C₅)cycloalkyl or a 3- to 8-membered heterocycle;
   R³ is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, —S(O)₂R⁵, —S(O)₂NR⁵R^{5′}, —S(O)R⁵, —S(O)NR⁵R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   or R² and R³, together with the atoms to which they are attached, can combine to form a 5 to 8-membered heterocyclic ring;
   or R¹ and R³, together with the atoms to which they are attached, can combine to form a 5 to 8-membered heterocyclic ring, optionally fused to an aryl or heteroaryl ring;
   R⁴ is —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   each R⁵ and R^{5′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, or —C(O)OR⁶, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁶, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, and —C(O)OR⁶;
   or two R⁵ on adjacent atoms together with the atoms to which they are attached form an aryl ring optionally substituted with one or more R⁶; or two R⁵ on adjacent atoms together with the atoms to which they are attached form a heteroaryl ring optionally substituted with one or more R⁶; or two R⁵ on adjacent atoms together with the atoms to which they are attached form a (C₃-C₈)cycloalkyl ring optionally substituted with one or more R⁶; or two R⁵ on adjacent atoms together with the atoms to which they are attached form a heterocycloalkyl ring optionally substituted with one or more R⁶;
   or two R^{5′} on adjacent atoms together with the atoms to which they are attached form an aryl ring optionally substituted with one or more R⁶; or two R^{5′} on adjacent atoms together with the atoms to which they are attached form a heteroaryl ring optionally substituted with one or more R⁶; or two R^{5′} on adjacent atoms together with the atoms to which they are attached form a (C₃—C₈)cycloalkyl ring optionally substituted with one or more R⁶; or two R^{5′} on adjacent atoms together with the atoms to which they are attached form a heterocycloalkyl ring optionally substituted with one or more R⁶;
   each R⁶ and R^{6′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, or —C(O)OR⁷, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁷, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, and —C(O)OR⁷;
   each R⁷ and R^{7′} is independently, at each occurrence, —H, —(C₁-C₆)alkyl, —(C₂-C₆)alkenyl, —(C₂-C₆)alkynyl, —(C₃-C₅)cycloalkyl, —(C₄-C₈)cycloalkenyl, heterocyclyl, aryl, heteroaryl, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, or —C(O)OH, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, and —C(O)OH;
   each R⁸, R^{8′}, R⁹, R^{9′}, R¹⁰, R^{10′}, R¹¹, and R^{11′} is independently, at each occurrence, —H, —(C₁-C₆)alkyl, —(C₂-C₆)alkenyl, —(C₂-C₆)alkynyl, —(C₃-C₈)cycloalkyl, or —(C₄-C₈)cycloalkenyl, wherein each alkyl, alkenyl, alkynyl, cycloalkyl and cycloalkenyl, is optionally substituted with one or more substituents selected from the group consisting of oxo, halogen, —CN, —R⁷, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, and —C(O)OR⁷;
and
t is 0, 1, 2, or 3.
2. The compound of embodiment 1, having Formula (Ia):

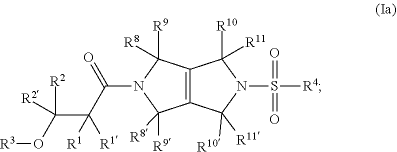

or a pharmaceutically acceptable salt thereof.
3. The compound of embodiment 1 or 2, having Formula (Ib):

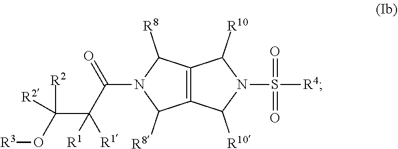

or a pharmaceutically acceptable salt thereof.
4. The compound of any one of embodiments 1-3, having Formula (Ic):

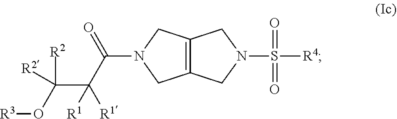

or a pharmaceutically acceptable salt thereof.
5. The compound of any one of embodiments 1-4, wherein R¹ and R^{1′} are each independently hydrogen, optionally substituted —(C₁-C₆)alkyl, optionally substituted aryl, or optionally substituted heteroaryl, or wherein R¹ and R^{1′}, taken together with the atoms to which they are attached, form an optionally substituted —(C₃-C₄)cycloalkyl.
6. The compound of any one of embodiments 1-5, wherein R² and R^{2′} are each independently hydrogen or optionally substituted —(C₁-C₆)alkyl.
7. The compound of any one of embodiments 1-6, wherein R³ is hydrogen or optionally substituted —(C₁-C₆)alkyl.
8. The compound of any one of embodiments 1-7, wherein R³ is hydrogen.
9. The compound of any one of embodiments 1-4, wherein R³ and one of R¹ or R^{1′}, taken together with the atoms to which they are attached, form an optionally substituted 5-6-membered heterocyclic ring.
10. The compound of any one of embodiments 1-4, wherein R¹ and R^{1′} are each independently hydrogen or optionally substituted phenyl or pyridyl; R² and R^{2′} are each independently hydrogen; and R³ is hydrogen or optionally substituted —(C₁-C₆)alkyl.
11. The compound of any one of embodiments 1-4, wherein R¹ and R^{1′} are each independently hydrogen or optionally substituted phenyl or pyridyl; R² and R^{2′} are each independently hydrogen; and R³ is hydrogen.
12. The compound of any one of embodiments 1-4, wherein R⁴ is optionally substituted aryl or heteroaryl.
13. The compound of embodiment 12, wherein the aryl or heteroaryl is optionally substituted with one or more substituents selected from the group consisting of oxo, halogen, —CN, —R⁵, —OR⁵, —S(O)₂NR⁵R^{5′}, —S(O)NR⁵R^{5′}, and —C(O)R⁵.
14. The compound of embodiment 12, wherein two R⁵ on adjacent atoms together with the atoms to which they are attached form an aryl ring optionally substituted with one or more R⁶.
15. The compound of embodiment 12, wherein two R⁵ on adjacent atoms together with the atoms to which they are attached form a heteroaryl ring optionally substituted with one or more R⁶.
16. The compound of embodiment 12, wherein two R⁵ on adjacent atoms together with the atoms to which they are attached form a (C₃-C₈)cycloalkyl ring optionally substituted with one or more R⁶.
17. The compound of embodiment 12, wherein two R⁵ on adjacent atoms together with the atoms to which they are attached form a heterocycloalkyl ring optionally substituted with one or more R⁶.
18. The compound of embodiment 1, wherein Y is —CR⁵R^{5′}—.
19. The compound of embodiment 1, wherein Y is —NR⁵(CR⁵R^{5′})ₜ—.
20. The compound of embodiment 1, wherein Y is —O—.
21. A compound of the Formula (Id-1):

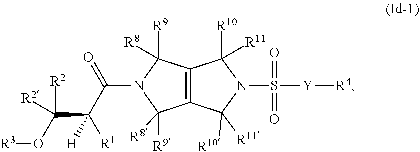

or a pharmaceutically acceptable salt thereof,
wherein:
   Y is a bond, —(CR⁵R^{5′})ₜ—, —NR⁵(CR⁵R^{5′})ₜ—, or —O—;
   R¹ is —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   each R² and R^{2′} is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵. —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵; or R² and R^{2′}, together with the atom to which they are attached, can combine to form —(C₃-C₅)cycloalkyl ring, heterocycle, (C₅-C₈)spirocycle or 5- to 8-membered spiroheterocycle;
   or R¹ and R², together with the atoms to which they are attached, can combine to form a —(C₃-C₅)cycloalkyl or a 3- to 8-membered heterocycle;
   R³ is independently —H, —(C₁-C₆)alkyl, —(C₂-C₆)alkenyl, —(C₂-C₆)alkynyl, —(C₃-C₈)cycloalkyl, —(C₄-C₅)cycloalkenyl, heterocyclyl, aryl, heteroaryl, —S(O)₂R⁵, —S(O)₂NR⁵R^{5′}, —S(O)R⁵, —S(O)NR⁵R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   or R² and R³, together with the atoms to which they are attached, can combine to form a 5 to 8-membered heterocyclic ring;
   or R¹ and R³, together with the atoms to which they are attached, can combine to form a 5 to 8-membered heterocyclic ring, optionally fused to an aryl or heteroaryl ring;
   R⁴ is —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   each R⁵ and R^{5′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, or —C(O)OR⁶, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁶, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, and —C(O)OR⁶;
   or two R⁵ on adjacent atoms together with the atoms to which they are attached form an aryl ring optionally substituted with one or more R⁶; or two R⁵ on adjacent atoms together with the atoms to which they are attached form a heteroaryl ring optionally substituted with one or more R⁶; or two R⁵ on adjacent atoms together with the atoms to which they are attached form a (C₃-C₈)cycloalkyl ring optionally substituted with one or more R⁶; or two R⁵ on adjacent atoms together with the atoms to which they are attached form a heterocycloalkyl ring optionally substituted with one or more R⁶;
   or two R^{5′} on adjacent atoms together with the atoms to which they are attached form an aryl ring optionally substituted with one or more R⁶; or two R^{5′} on adjacent atoms together with the atoms to which they are attached form a heteroaryl ring optionally substituted with one or more R⁶; or two R^{5′} on adjacent atoms together with the atoms to which they are attached form a (C₃-C₈)cycloalkyl ring optionally substituted with one or more R⁶; or two R^{5′} on adjacent atoms together with the atoms to which they are attached form a heterocycloalkyl ring optionally substituted with one or more R⁶;
   each R⁶ and R^{6′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, or —C(O)OR⁷, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁷, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{T}, —NR⁷S(O)R^{7′}, —C(O)R⁷, and —C(O)OR⁷;
   each R⁷ and R^{7′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, or —C(O)OH, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, and —C(O)OH;
   each R⁸, R^{8′}, R⁹, R^{9′}, R¹⁰, R^{10′}, R¹¹, and R^{11′} is independently, at each occurrence, —H, —(C₁-C₆)alkyl, —(C₂-C₆)alkenyl, —(C₂-C₆)alkynyl, —(C₃-C₅)cycloalkyl, or —(C₄-C₅)cycloalkenyl, wherein each alkyl, alkenyl, alkynyl, cycloalkyl and cycloalkenyl, is optionally substituted with one or more substituents selected from the group consisting of oxo, halogen, —CN, —R⁷, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, and —C(O)OR⁷;
and
t is 0, 1, 2, or 3.
22. The compound of embodiment 21, having Formula (Ia-1):

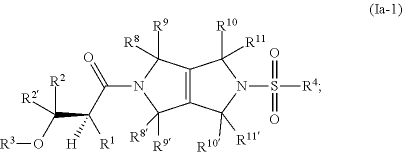

or a pharmaceutically acceptable salt thereof.
23. The compound of embodiments 21 or 22, having Formula (Ib-1):

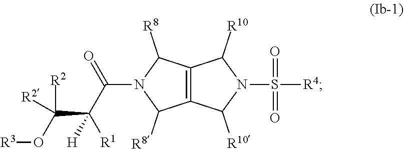

or a pharmaceutically acceptable salt thereof.
24. The compound of any one of embodiments 21-23, having Formula (Ic-1):

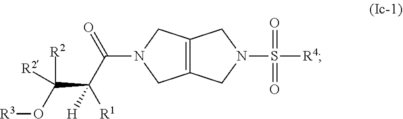

or a pharmaceutically acceptable salt thereof.
25. The compound of any one of embodiments 21-24, wherein R¹ is optionally substituted —(C₁-C₆)alkyl, optionally substituted aryl, or optionally substituted heteroaryl.
26. The compound of any one of embodiments 21-25, wherein R² and R^{2′} are each independently hydrogen or optionally substituted —(C₁-C₆)alkyl.
27. The compound of any one of embodiments 21-26, wherein R³ is hydrogen or optionally substituted —(C₁-C₆)alkyl.
28. The compound of any one of embodiments 21-27, wherein R³ is hydrogen.
29. The compound of any one of embodiments 21-24, wherein R³ and R¹ taken together with the atoms to which they are attached, form an optionally substituted 5-6-membered heterocyclic ring.
30. The compound of any one of embodiments 21-24, wherein R¹ is optionally substituted phenyl or pyridyl; R² and R^{2′} are each independently hydrogen; and R³ is hydrogen or optionally substituted —(C₁-C₆)alkyl.
31. The compound of any one of embodiments 21-24, wherein R¹ is optionally substituted phenyl or pyridyl; R² and R^{2′} are each independently hydrogen; and R³ is hydrogen.
32. The compound of any one of embodiments 21-31, wherein R⁴ is optionally substituted aryl or heteroaryl.
33. The compound of embodiment 32, wherein the aryl or heteroaryl is optionally substituted with one or more substituents selected from the group consisting of oxo, halogen, —CN, —R⁵, —OR⁵, —S(O)₂NR⁵R^{5′}, —S(O)NR⁵R^{5′}, and —C(O)R⁵.
34. The compound of embodiment 32, wherein two R⁵ on adjacent atoms together with the atoms to which they are attached form an aryl ring optionally substituted with one or more R⁶.
35. The compound of embodiment 32, wherein two R⁵ on adjacent atoms together with the atoms to which they are attached form a heteroaryl ring optionally substituted with one or more R⁶.
36. The compound of embodiment 32, wherein two R⁵ on adjacent atoms together with the atoms to which they are attached form a (C₃-C₈)cycloalkyl ring optionally substituted with one or more R⁶.
37. The compound of embodiment 32, wherein two R⁵ on adjacent atoms together with the atoms to which they are attached form a heterocycloalkyl ring optionally substituted with one or more R⁶.
38. The compound of embodiment 21, wherein Y is —CR⁵R^{5′}—.
39. The compound of embodiment 21, wherein Y is —NR⁵(CR⁵R^{5′})ₜ—.
40. The compound of embodiment 21, wherein Y is —O—.
41. A compound of the Formula (Id-2):

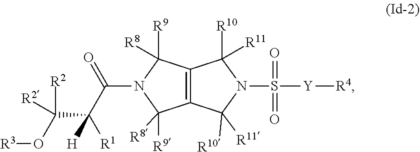

or a pharmaceutically acceptable salt thereof,
wherein:
   Y is a bond, —(CR⁵R^{5′})ₜ—, —NR⁵(CR⁵R^{5′})ₜ—, or —O—;
   R¹ is —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   each R² and R^{2′} is independently —H, —(C₁-C₆)alkyl, —(C₂-C₆)alkenyl, —(C₂-C₆)alkynyl, —(C₃-C₅)cycloalkyl, —(C₄-C₅)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵; or R² and R^{2′}, together with the atom to which they are attached, can combine to form —(C₃-C₈)cycloalkyl ring, heterocycle, (C₅-C₈)spirocycle or 5- to 8-membered spiroheterocycle;
   or R¹ and R², together with the atoms to which they are attached, can combine to form a —(C₃-C₅)cycloalkyl or a 3- to 8-membered heterocycle;
   R³ is independently —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, —S(O)₂R⁵, —S(O)₂NR⁵R^{5′}, —S(O)R⁵, —S(O)NR⁵R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   or R² and R³, together with the atoms to which they are attached, can combine to form a 5 to 8-membered heterocyclic ring;
   or R¹ and R³, together with the atoms to which they are attached, can combine to form a 5 to 8-membered heterocyclic ring, optionally fused to an aryl or heteroaryl ring;
   R⁴ is —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, or —C(O)OR⁵, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁵, —OR⁵, —SR⁵, —NO₂, —NR⁵R^{5′}, —S(O)₂R⁵, —S(O)₂NR⁵R^{5′}, —S(O)R⁵, —S(O)NR⁵R^{5′}, —NR⁵S(O)₂R^{5′}, —NR⁵S(O)R^{5′}, —C(O)R⁵, and —C(O)OR⁵;
   each R⁵ and R^{5′} is independently, at each occurrence, —H, —(C₁-C₆)alkyl, —(C₂-C₆)alkenyl, —(C₂-C₆)alkynyl, —(C₃-C₅)cycloalkyl, —(C₄-C₈)cycloalkenyl, heterocyclyl, aryl, heteroaryl, halogen, —CN, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, or —C(O)OR⁶, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁶, —OR⁶, —SR⁶, —NO₂, —NR⁶R^{6′}, —S(O)₂R⁶, —S(O)₂NR⁶R^{6′}, —S(O)R⁶, —S(O)NR⁶R^{6′}, —NR⁶S(O)₂R^{6′}, —NR⁶S(O)R^{6′}, —C(O)R⁶, and —C(O)OR⁶;
   or two R⁵ on adjacent atoms together with the atoms to which they are attached form an aryl ring optionally substituted with one or more R⁶; or two R⁵ on adjacent atoms together with the atoms to which they are attached form a heteroaryl ring optionally substituted with one or more R⁶; or two R⁵ on adjacent atoms together with the atoms to which they are attached form a (C₃-C₈)cycloalkyl ring optionally substituted with one or more R⁶; or two R⁵ on adjacent atoms together with the atoms to which they are attached form a heterocycloalkyl ring optionally substituted with one or more R⁶;
   or two R^{5′} on adjacent atoms together with the atoms to which they are attached form an aryl ring optionally substituted with one or more R⁶; or two R^{5′} on adjacent atoms together with the atoms to which they are attached form a heteroaryl ring optionally substituted with one or more R⁶; or two R^{5′} on adjacent atoms together with the atoms to which they are attached form a (C₃-C₈)cycloalkyl ring optionally substituted with one or more R⁶; or two R^{5′} on adjacent atoms together with the atoms to which they are attached form a heterocycloalkyl ring optionally substituted with one or more R⁶;
   each R⁶ and R^{6′} is independently, at each occurrence, —H, —(C₁-C₆)alkyl, —(C₂-C₆)alkenyl, —(C₂-C₆)alkynyl, —(C₃-C₈)cycloalkyl, —(C₄-C₈)cycloalkenyl, heterocyclyl, aryl, heteroaryl, halogen, —CN, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R^{7′}, —C(O)R⁷, or —C(O)OR⁷, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —R⁷, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{T}, —NR⁷S(O)R^{7′}, —C(O)R⁷, and —C(O)OR⁷;
   each R⁷ and R^{7′} is independently, at each occurrence, —H, —(C₁-C₆)alkyl, —(C₂-C₆)alkenyl, —(C₂-C₆)alkynyl, —(C₃-C₅)cycloalkyl, —(C₄-C₈)cycloalkenyl, heterocyclyl, aryl, heteroaryl, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, or —C(O)OH, wherein each alkyl, alkenyl, alkynyl, cycloalkyl, cycloalkenyl, heterocyclyl, aryl, or heteroaryl is optionally substituted with one or more substituents selected from the group consisting of oxo, halogen, —CN, —OH, —SH, —NO₂, —NH₂, —S(O)₂H, —S(O)₂NH₂, —S(O)H, —S(O)NH₂, —NHS(O)₂H, —NHS(O)H, —C(O)H, and —C(O)OH;
   each R⁸, R^{8′}, R⁹, R^{9′}, R¹⁰, R^{10′}, R¹¹, and R^{11′} is independently, at each occurrence, —H, —(C₁-C₆)alkyl, —(C₂-C₆)alkenyl, —(C₂-C₆)alkynyl, —(C₃-C₈)cycloalkyl, or —(C₄-C₈)cycloalkenyl, wherein each alkyl, alkenyl, alkynyl, cycloalkyl and cycloalkenyl, is optionally substituted with one or more substituents selected from the group consisting of oxo, halogen, —CN, —R⁷, —OR⁷, —SR⁷, —NO₂, —NR⁷R^{7′}, —S(O)₂R⁷, —S(O)₂NR⁷R^{7′}, —S(O)R⁷, —S(O)NR⁷R^{7′}, —NR⁷S(O)₂R^{7′}, —NR⁷S(O)R⁷—, —C(O)R⁷, and —C(O)OR⁷;
and
t is 0, 1, 2, or 3.
42. The compound of embodiment 41, having Formula (la-2):

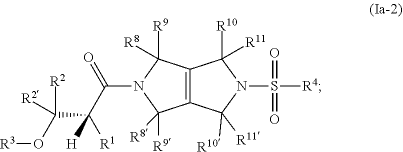

or a pharmaceutically acceptable salt thereof.
43. The compound of embodiment 41 or 42, having Formula (Ib-2):

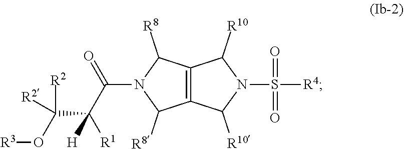

or a pharmaceutically acceptable salt thereof.
44. The compound of any one of embodiments 41-43, having Formula (Ic-2):

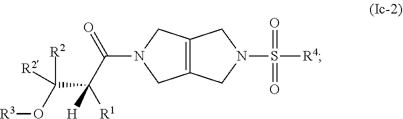

or a pharmaceutically acceptable salt thereof.
45. The compound of any one of embodiments 41-44, wherein R¹ is optionally substituted —(C₁-C₆)alkyl, optionally substituted aryl, or optionally substituted heteroaryl.
46. The compound of any one of embodiments 41-45, wherein R² and R^{2′} are each independently hydrogen or optionally substituted —(C₁-C₆)alkyl.
47. The compound of any one of embodiments 41-46, wherein R³ is hydrogen or optionally substituted —(C₁-C₆)alkyl.
48. The compound of any one of embodiments 41-47, wherein R³ is hydrogen.
49. The compound of any one of embodiments 41-44, wherein R³ and R¹ taken together with the atoms to which they are attached, form an optionally substituted 5-6-membered heterocyclic ring.
50. The compound of any one of embodiments 41-44, wherein R¹ is optionally substituted phenyl or pyridyl; R² and R^{2′} are each independently hydrogen; and R³ is hydrogen or optionally substituted —(C₁-C₆)alkyl.
51. The compound of any one of embodiments 41-44, wherein R¹ is optionally substituted phenyl or pyridyl; R² and R^{2′} are each independently hydrogen; and R³ is hydrogen.
52. The compound of any one of embodiments 41-51, wherein R⁴ is optionally substituted aryl or heteroaryl.
53. The compound of embodiment 52, wherein the aryl or heteroaryl is optionally substituted with one or more substituents selected from the group consisting of oxo, halogen, —CN, —R⁵, —OR⁵, —S(O)₂NR⁵R^{5′}, —S(O)NR⁵R^{5′}, and —C(O)R⁵.
54. The compound of embodiment 52, wherein two R⁵ on adjacent atoms together with the atoms to which they are attached form an aryl ring optionally substituted with one or more R⁶.
55. The compound of embodiment 52, wherein two R⁵ on adjacent atoms together with the atoms to which they are attached form a heteroaryl ring optionally substituted with one or more R⁶.
56. The compound of embodiment 52, wherein two R⁵ on adjacent atoms together with the atoms to which they are attached form a (C₃-C₈)cycloalkyl ring optionally substituted with one or more R⁶.
57. The compound of embodiment 52, wherein two R⁵ on adjacent atoms together with the atoms to which they are attached form a heterocycloalkyl ring optionally substituted with one or more R⁶.
58. The compound of embodiment 41, wherein Y is —CR⁵R^{5′}—.
59. The compound of embodiment 41, wherein Y is —NR⁵(CR⁵R^{5′})ₜ—.
60. The compound of embodiment 41, wherein Y is —O—.
61. A compound selected from the group consisting of:

| Example | Structure | Name |
|---|---|---|
| 1 | 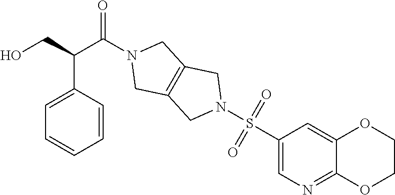 [SMILES: O=C([C@H](CO)c1ccccc1)N1CC2=C(C1)CN(S(=O)(=O)c1cnc3c(c1)OCCO3)C2] | (S)-1-(5-((2,3-dihydro-[1,4]dioxino[2,3- b]pyridin-7-yl)sulfonyl)-3,4,5,6- tetrahydropyrrolo[3,4-c]pyrrol-2(1H)- yl)-3-hydroxy-2-phenylpropan-1-one |
| 2 | 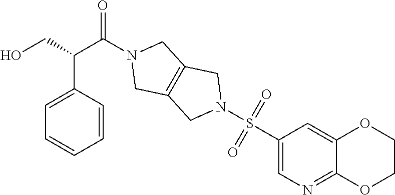 [SMILES: O=C([C@@H](CO)c1ccccc1)N1CC2=C(C1)CN(S(=O)(=O)c1cnc3c(c1)OCCO3)C2] | (R)-1-(5-((2,3-dihydro-[1,4]dioxino[2,3- b]pyridin-7-yl)sulfonyl)-3,4,5,6- tetrahydropyrrolo[3,4-c]pyrrol-2(1H)- yl)-3-hydroxy-2-phenylpropan-1-one |
| 3 | 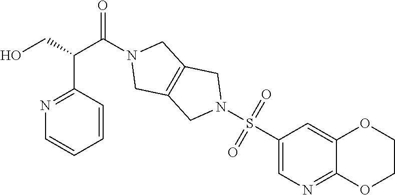 [SMILES: O=C([C@@H](CO)c1ccccn1)N1CC2=C(C1)CN(S(=O)(=O)c1cnc3c(c1)OCCO3)C2] | (R)-1-(5-((2,3-dihydro-[1,4]dioxino[2,3- b]pyridin-7-yl)sulfonyl)-3,4,5,6- tetrahydropyrrolo[3,4-c]pyrrol-2(1H)- yl)-3-hydroxy-2-(pyridin-2-yl)propan-1- one |
| 4 | 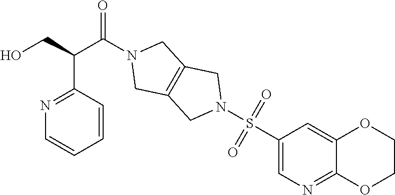 [SMILES: O=C([C@H](CO)c1ccccn1)N1CC2=C(C1)CN(S(=O)(=O)c1cnc3c(c1)OCCO3)C2] | (S)-1-(5-((2,3-dihydro-[1,4]dioxino[2,3- b]pyridin-7-yl)sulfonyl)-3,4,5,6- tetrahydropyrrolo[3,4-c]pyrrol-2(1H)- yl)-3-hydroxy-2-(pyridin-2-yl)propan-1- one |
| 5 | 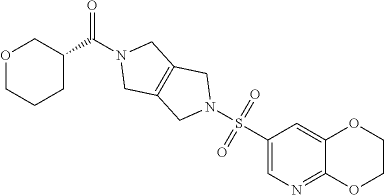 [SMILES: O=C([C@@H]1CCCOC1)N1CC2=C(C1)CN(S(=O)(=O)c1cnc3c(c1)OCCO3)C2] | (R)-(5-((2,3-dihydro-[1,4]dioxino[2,3- b]pyridin-7-yl)sulfonyl)-3,4,5,6- tetrahydropyrrolo[3,4-c]pyrrol-2(1H)- yl)(tetrahydro-2H-pyran-3-yl)methanone |
| 6 | 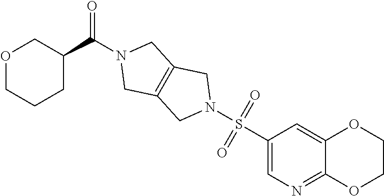 [SMILES: O=C([C@H]1CCCOC1)N1CC2=C(C1)CN(S(=O)(=O)c1cnc3c(c1)OCCO3)C2] | (S)-(5-((2,3-dihydro-[1,4]dioxino[2,3- b]pyridin-7-yl)sulfonyl)-3,4,5,6- tetrahydropyrrolo[3,4-c]pyrrol-2(1H)- yl)(tetrahydro-2H-pyran-3-yl)methanone |
| 7 | 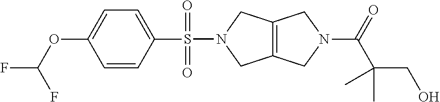 [SMILES: CC(C)(CO)C(=O)N1CC2=C(C1)CN(S(=O)(=O)c1ccc(OC(F)F)cc1)C2] | 1-(5-((4- (difluoromethoxy)phenyl)sulfonyl)- 3,4,5,6-tetrahydropyrrolo[3,4-c]pyrrol- 2(1H)-yl)-3-hydroxy-2,2- dimethylpropan-1-one |
| 8 | 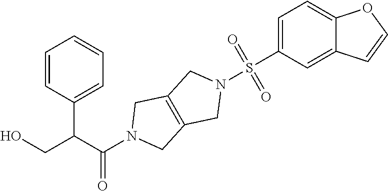 [SMILES: O=C(C(CO)c1ccccc1)N1CC2=C(C1)CN(S(=O)(=O)c1ccc3occc3c1)C2] | 1-(5-(benzofuran-5-ylsulfonyl)-3,4,5,6- tetrahydropyrrolo[3,4-c]pyrrol-2(1H)- yl)-3-hydroxy-2-phenylpropan-1-one |
| 9 | 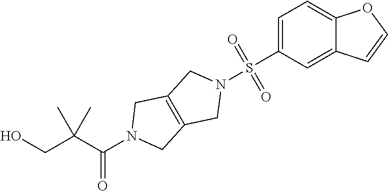 [SMILES: CC(C)(CO)C(=O)N1CC2=C(C1)CN(S(=O)(=O)c1ccc3occc3c1)C2] | 1-(5-(benzofuran-5-ylsulfonyl)-3,4,5,6- tetrahydropyrrolo[3,4-c]pyrrol-2(1H)- yl)-3-hydroxy-2,2-dimethylpropan-1- one |
| 10 | 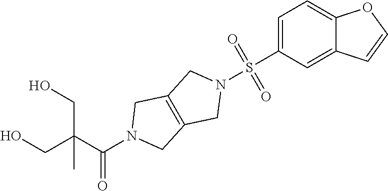 [SMILES: CC(CO)(CO)C(=O)N1CC2=C(C1)CN(S(=O)(=O)c1ccc3occc3c1)C2] | 1-(5-(benzofuran-5-ylsulfonyl)-3,4,5,6- tetrahydropyrrolo[3,4-c]pyrrol-2(1H)- yl)-3-hydroxy-2-(hydroxymethyl)-2- methylpropan-1-one |
| 11 | 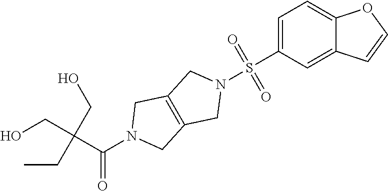 [SMILES: CCC(CO)(CO)C(=O)N1CC2=C(C1)CN(S(=O)(=O)c1ccc3occc3c1)C2] | 1-(5-(benzofuran-5-ylsulfonyl)-3,4,5,6- tetrahydropyrrolo[3,4-c]pyrrol-2(1H)- yl)-2,2-bis(hydroxymethyl)butan-1-one |
| 12 | 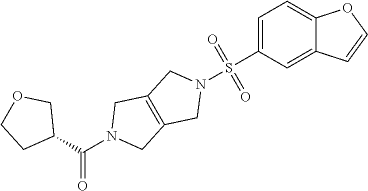 [SMILES: O=C([C@@H]1CCOC1)N1CC2=C(C1)CN(S(=O)(=O)c1ccc3occc3c1)C2] | (R)-(5-(benzofuran-5-ylsulfonyl)- 3,4,5,6-tetrahydropyrrolo[3,4-c]pyrrol- 2(1H)-yl)(tetrahydrofuran-3- yl)methanone |
| 13 | 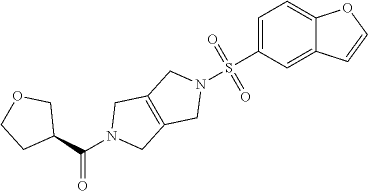 [SMILES: O=C([C@H]1CCOC1)N1CC2=C(C1)CN(S(=O)(=O)c1ccc3occc3c1)C2] | (S)-(5-(benzofuran-5-ylsulfonyl)- 3,4,5,6-tetrahydropyrrolo[3,4-c]pyrrol- 2(1H)-yl)(tetrahydrofuran-3- yl)methanone |
| 14 | 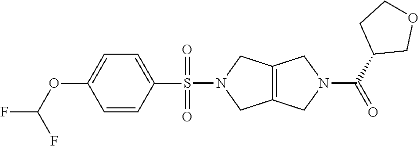 [SMILES: O=C([C@@H]1CCOC1)N1CC2=C(C1)CN(S(=O)(=O)c1ccc(OC(F)F)cc1)C2] | (R)-(5-((4- (difluoromethoxy)phenyl)sulfonyl)- 3,4,5,6-tetrahydropyrrolo[3,4-c]pyrrol- 2(1H)-yl)(tetrahydrofuran-3- yl)methanone |
| 15 | 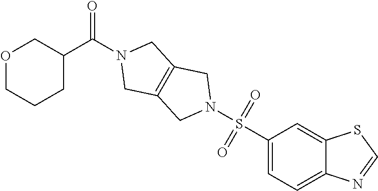 [SMILES: O=C(C1CCCOC1)N1CC2=C(C1)CN(S(=O)(=O)c1ccc3ncsc3c1)C2] | (5-(benzo[d]thiazol-6-ylsulfonyl)- 3,4,5,6-tetrahydropyrrolo[3,4-c]pyrrol- 2(1H)-yl)(tetrahydro-2H-pyran-3- yl)methanone |
| 16 | 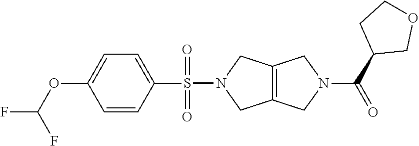 [SMILES: O=C([C@H]1CCOC1)N1CC2=C(C1)CN(S(=O)(=O)c1ccc(OC(F)F)cc1)C2] | (S)-(5-((4- (difluoromethoxy)phenyl)sulfonyl)- 3,4,5,6-tetrahydropyrrolo[3,4-c]pyrrol- 2(1H)-yl)(tetrahydrofuran-3- yl)methanone |
| 17 | 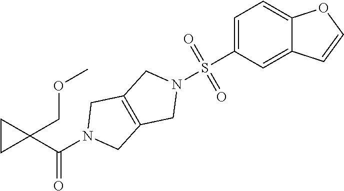 [SMILES: COCC1(C(=O)N2CC3=C(C2)CN(S(=O)(=O)c2ccc4occc4c2)C3)CC1] | (5-(benzofuran-5-ylsulfonyl)-3,4,5,6- tetrahydropyrrolo[3,4-c]pyrrol-2(1H)- yl)(1- (methoxymethyl)cyclopropyl)methanone |
| 18 | 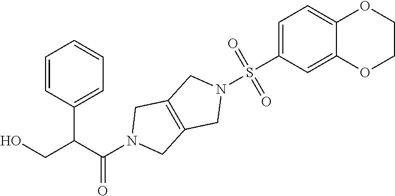 [SMILES: O=C(C(CO)c1ccccc1)N1CC2=C(C1)CN(S(=O)(=O)c1ccc3c(c1)OCCO3)C2] | 1-(5-((2,3-dihydrobenzo[b][1,4]dioxin- 6-yl)sulfonyl)-3,4,5,6- tetrahydropyrrolo[3,4-c]pyrrol-2(1H)- yl)-3-hydroxy-2-phenylpropan-1-one |
| 19 | 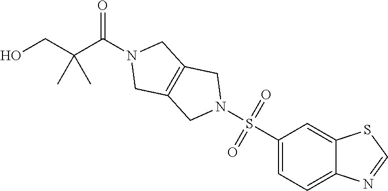 [SMILES: CC(C)(CO)C(=O)N1CC2=C(C1)CN(S(=O)(=O)c1ccc3ncsc3c1)C2] | 1-(5-(benzo[d]thiazol-6-ylsulfonyl)- 3,4,5,6-tetrahydropyrrolo[3,4-c]pyrrol- 2(1H)-yl)-3-hydroxy-2,2- dimethylpropan-1-one |
| 20 | 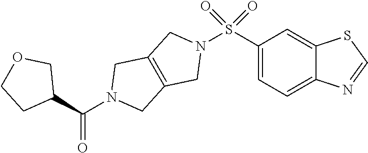 | (R)-(5-(benzo[d]thiazol-6-ylsulfonyl)- 3,4,5,6-tetrahydropyrrolo[3,4-c]pyrrol- 2(1H)-yl)(tetrahydrofuran-3- yl)methanone |
| 21 | 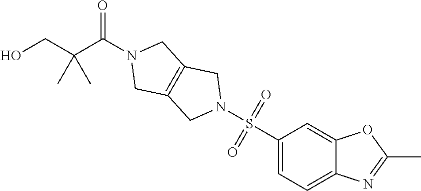 [SMILES: Cc1nc2ccc(S(=O)(=O)N3CC4=C(CN(C(=O)C(C)(C)CO)C4)C3)cc2o1] | 3-hydroxy-2,2-dimethyl-1-(5-((2- methylbenzo[d]oxazol-6-yl)sulfonyl)- 3,4,5,6-tetrahydropyrrolo[3,4-c]pyrrol- 2(1H)-yl)propan-1-one |
| 22 | 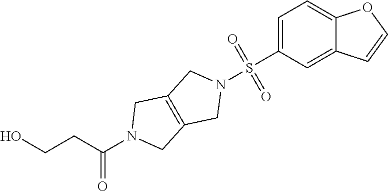 [SMILES: O=C(CCO)N1CC2=C(C1)CN(S(=O)(=O)c1ccc3occc3c1)C2] | 1-(5-(benzofuran-5-ylsulfonyl)-3,4,5,6- tetrahydropyrrolo[3,4-c]pyrrol-2(1H)- yl)-3-hydroxypropan-1-one |
| 23 | 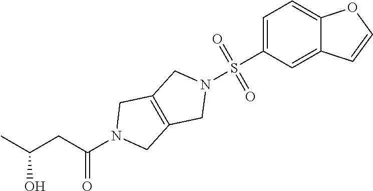 [SMILES: C[C@@H](O)CC(=O)N1CC2=C(C1)CN(S(=O)(=O)c1ccc3occc3c1)C2] | (R)-1-(5-(benzofuran-5-ylsulfonyl)- 3,4,5,6-tetrahydropyrrolo[3,4-c]pyrrol- 2(1H)-yl)-3-hydroxybutan-1-one |
| 24 | 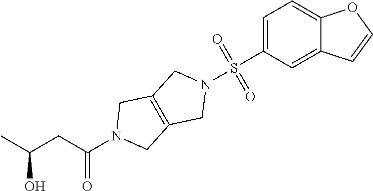 [SMILES: C[C@H](O)CC(=O)N1CC2=C(C1)CN(S(=O)(=O)c1ccc3occc3c1)C2] | (S)-1-(5-(benzofuran-5-ylsulfonyl)- 3,4,5,6-tetrahydropyrrolo[3,4-c]pyrrol- 2(1H)-yl)-3-hydroxybutan-1-one |
| 25 | 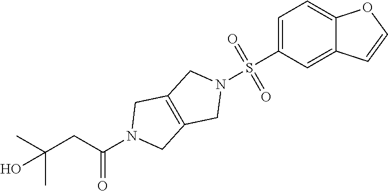 [SMILES: CC(C)(O)CC(=O)N1CC2=C(C1)CN(S(=O)(=O)c1ccc3occc3c1)C2] | 1-(5-(benzofuran-5-ylsulfonyl)-3,4,5,6- tetrahydropyrrolo[3,4-c]pyrrol-2(1H)- yl)-3-hydroxy-3-methylbutan-1-one |
| 26 | 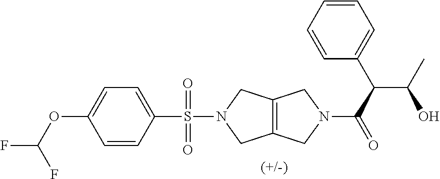 [SMILES: C[C@@H](O)[C@@H](C(=O)N1CC2=C(C1)CN(S(=O)(=O)c1ccc(OC(F)F)cc1)C2)c1ccccc1] | (2S,3R and 2R,3S)-1-(5-((4- (difluoromethoxy)phenyl)sulfonyl)- 3,4,5,6-tetrahydropyrrolo[3,4-c]pyrrol- 2(1H)-yl)-3-hydroxy-2-phenylbutan-1- one |
| 27 | 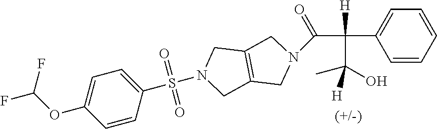 [SMILES: C[C@@H](O)[C@H](C(=O)N1CC2=C(C1)CN(S(=O)(=O)c1ccc(OC(F)F)cc1)C2)c1ccccc1] | (2R,3R and 2S,3S)-1-(5-((4- (difluoromethoxy)phenyl)sulfonyl)- 3,4,5,6-tetrahydropyrrolo[3,4-c]pyrrol- 2(1H)-yl)-3-hydroxy-2-phenylbutan-1- one |
| 28 | 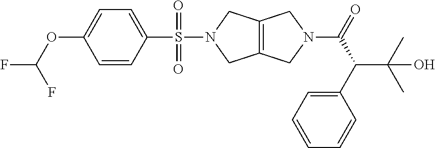 [SMILES: CC(C)(O)[C@@H](C(=O)N1CC2=C(C1)CN(S(=O)(=O)c1ccc(OC(F)F)cc1)C2)c1ccccc1] | (S)-1-(5-((4- (difluoromethoxy)phenyl)sulfonyl)- 3,4,5,6-tetrahydropyrrolo[3,4-c]pyrrol- 2(1H)-yl)-3-hydroxy-3-methyl-2- phenylbutan-1-one |
| 29 | 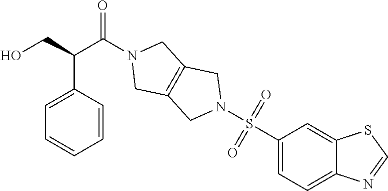 [SMILES: O=C([C@H](CO)c1ccccc1)N1CC2=C(C1)CN(S(=O)(=O)c1ccc3ncsc3c1)C2] | (S)-1-(5-(benzo[d]thiazol-6-ylsulfonyl)- 3,4,5,6-tetrahydropyrrolo[3,4-c]pyrrol- 2(1H)-yl)-3-hydroxy-2-phenylpropan-1- one |
| 30 | 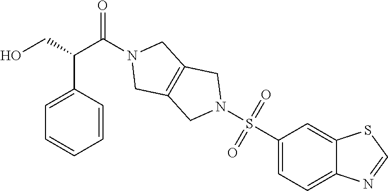 [SMILES: O=C([C@@H](CO)c1ccccc1)N1CC2=C(C1)CN(S(=O)(=O)c1ccc3ncsc3c1)C2] | (R)-1-(5-(benzo[d]thiazol-6-ylsulfonyl)- 3,4,5,6-tetrahydropyrrolo[3,4-c]pyrrol- 2(1H)-yl)-3-hydroxy-2-phenylpropan-1- one |
| 31 | 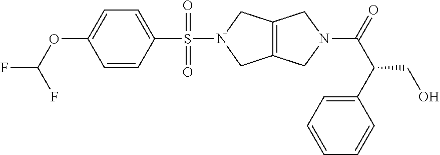 [SMILES: O=C([C@H](CO)c1ccccc1)N1CC2=C(C1)CN(S(=O)(=O)c1ccc(OC(F)F)cc1)C2] | (S)-1-(5-((4- (difluoromethoxy)phenyl)sulfonyl)- 3,4,5,6-tetrahydropyrrolo[3,4-c]pyrrol- 2(1H)-yl)-3-hydroxy-2-phenylpropan-1- one |
| 32 | 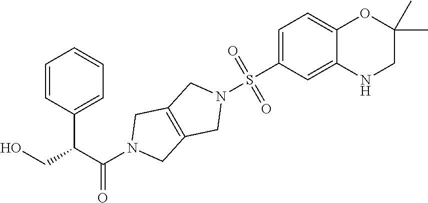 [SMILES: CC1(C)CNc2cc(S(=O)(=O)N3CC4=C(CN(C(=O)[C@H](CO)c5ccccc5)C4)C3)ccc2O1] | (S)-1-(5-((2,2-dimethyl-3,4-dihydro-2H- benzo[b][1,4]oxazin-6-yl)sulfonyl)- 3,4,5,6-tetrahydropyrrolo[3,4-c]pyrrol- 2(1H)-yl)-3-hydroxy-2-phenylpropan-1- one |
| 33 | 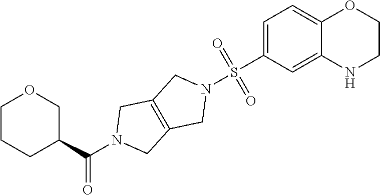 [SMILES: O=C([C@H]1CCCOC1)N1CC2=C(C1)CN(S(=O)(=O)c1ccc3c(c1)NCCO3)C2] | (S)-(5-((3,4-dihydro-2H- benzo[b][1,4]oxazin-6-yl)sulfonyl)- 3,4,5,6-tetrahydropyrrolo[3,4-c]pyrrol- 2(1H)-yl)(tetrahydro-2H-pyran-3- yl)methanone |
| 34 | 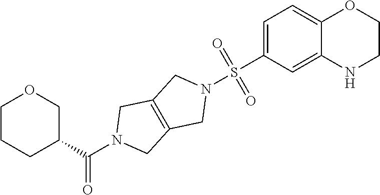 [SMILES: O=C([C@@H]1CCCOC1)N1CC2=C(C1)CN(S(=O)(=O)c1ccc3c(c1)NCCO3)C2] | (R)-(5-((3,4-dihydro-2H- benzo[b][1,4]oxazin-6-yl)sulfonyl)- 3,4,5,6-tetrahydropyrrolo[3,4-c]pyrrol- 2(1H)-yl)(tetrahydro-2H-pyran-3- yl)methanone |
| 35 | 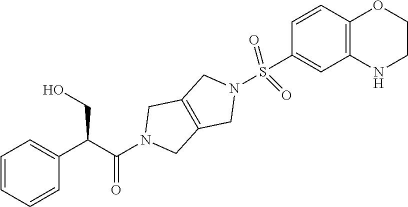 [SMILES: O=C([C@H](CO)c1ccccc1)N1CC2=C(C1)CN(S(=O)(=O)c1ccc3c(c1)NCCO3)C2] | (S)-1-(5-((3,4-dihydro-2H- benzo[b][1,4]oxazin-6-yl)sulfonyl)- 3,4,5,6-tetrahydropyrrolo[3,4-c]pyrrol- 2(1H)-yl)-3-hydroxy-2-phenylpropan-1- one |
| 36 | 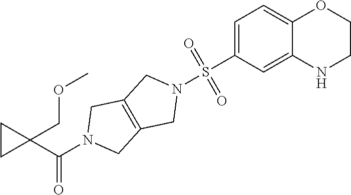 [SMILES: COCC1(C(=O)N2CC3=C(C2)CN(S(=O)(=O)c2ccc4c(c2)NCCO4)C3)CC1] | (5-((3,4-dihydro-2H- benzo[b][1,4]oxazin-6-yl)sulfonyl)- 3,4,5,6-tetrahydropyrrolo[3,4-c]pyrrol- 2(1H)-yl)(1- (methoxymethyl)cyclopropyl)methanone |
| 37 | 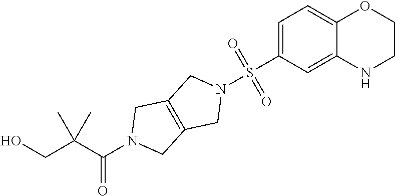 [SMILES: CC(C)(CO)C(=O)N1CC2=C(C1)CN(S(=O)(=O)c1ccc3c(c1)NCCO3)C2] | 1-(5-((3,4-dihydro-2H- benzo[b][1,4]oxazin-6-yl)sulfonyl)- 3,4,5,6-tetrahydropyrrolo[3,4-c]pyrrol- 2(1H)-yl)-3-hydroxy-2,2- dimethylpropan-1-one |
| 38 | 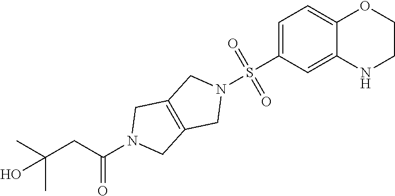 [SMILES: CC(C)(O)CC(=O)N1CC2=C(C1)CN(S(=O)(=O)c1ccc3c(c1)NCCO3)C2] | 1-(5-((3,4-dihydro-2H- benzo[b][1,4]oxazin-6-yl)sulfonyl)- 3,4,5,6-tetrahydropyrrolo[3,4-c]pyrrol- 2(1H)-yl)-3-hydroxy-3-methylbutan-1- one |
| 39 | 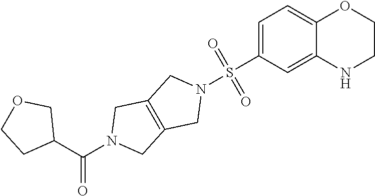 [SMILES: O=C(C1CCOC1)N1CC2=C(C1)CN(S(=O)(=O)c1ccc3c(c1)NCCO3)C2] | (5-((3,4-dihydro-2H- benzo[b][1,4]oxazin-6-yl)sulfonyl)- 3,4,5,6-tetrahydropyrrolo[3,4-c]pyrrol- 2(1H)-yl)(tetrahydrofuran-3- yl)methanone |
| 40 | 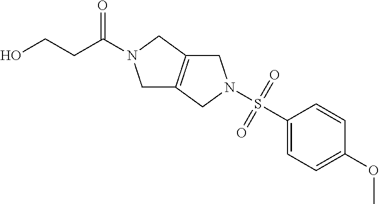 [SMILES: COc1ccc(S(=O)(=O)N2CC3=C(CN(C(=O)CCO)C3)C2)cc1] | 3-hydroxy-1-(5-((4- methoxyphenyl)sulfonyl)-3,4,5,6- tetrahydropyrrolo[3,4-c]pyrrol-2(1H)- yl)propan-1-one |
| 41 | 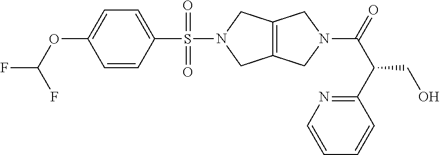 [SMILES: O=C([C@H](CO)c1ccccn1)N1CC2=C(C1)CN(S(=O)(=O)c1ccc(OC(F)F)cc1)C2] | (S)-1-(5-((4- (difluoromethoxy)phenyl)sulfonyl)- 3,4,5,6-tetrahydropyrrolo[3,4-c]pyrrol- 2(1H)-yl)-3-hydroxy-2-(pyridin-2- yl)propan-1-one |
| 42 | 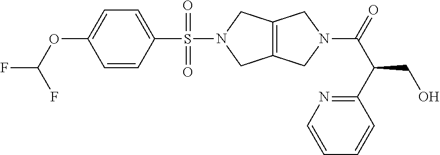 [SMILES: O=C([C@@H](CO)c1ccccn1)N1CC2=C(C1)CN(S(=O)(=O)c1ccc(OC(F)F)cc1)C2] | (R)-1-(5-((4- (difluoromethoxy)phenyl)sulfonyl)- 3,4,5,6-tetrahydropyrrolo[3,4-c]pyrrol- 2(1H)-yl)-3-hydroxy-2-(pyridin-2- yl)propan-1-one |
| 43 | 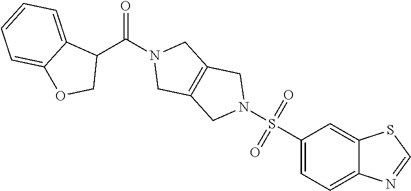 [SMILES: O=C(C1COc2ccccc21)N1CC2=C(C1)CN(S(=O)(=O)c1ccc3ncsc3c1)C2] | (5-(benzo[d]thiazol-6-ylsulfonyl)- 3,4,5,6-tetrahydropyrrolo[3,4-c]pyrrol- 2(1H)-yl)(2,3-dihydrobenzofuran-3- yl)methanone |
| 44 | 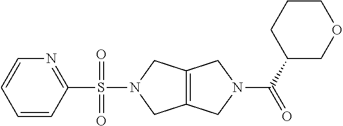 [SMILES: O=C([C@@H]1CCCOC1)N1CC2=C(C1)CN(S(=O)(=O)c1ccccn1)C2] | (R)-(5-(pyridin-2-ylsulfonyl)-3,4,5,6- tetrahydropyrrolo[3,4-c]pyrrol-2(1H)- yl)(tetrahydro-2H-pyran-3-yl)methanone |
| 45 | 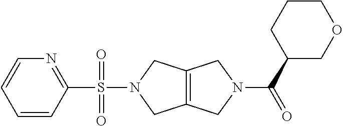 [SMILES: O=C([C@H]1CCCOC1)N1CC2=C(C1)CN(S(=O)(=O)c1ccccn1)C2] | (S)-(5-(pyridin-2-ylsulfonyl)-3,4,5,6- tetrahydropyrrolo[3,4-c]pyrrol-2(1H)- yl)(tetrahydro-2H-pyran-3-yl)methanone |
| 46 | 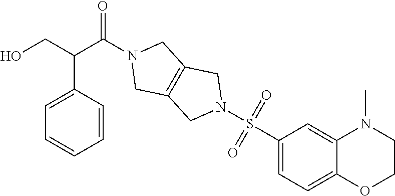 [SMILES: CN1CCOc2ccc(S(=O)(=O)N3CC4=C(CN(C(=O)C(CO)c5ccccc5)C4)C3)cc21] | 3-hydroxy-1-(5-((4-methyl-3,4-dihydro- 2H-benzo[b][1,4]oxazin-6-yl)sulfonyl)- 3,4,5,6-tetrahydropyrrolo[3,4-c]pyrrol- 2(1H)-yl)-2-phenylpropan-1-one |
| 47 | 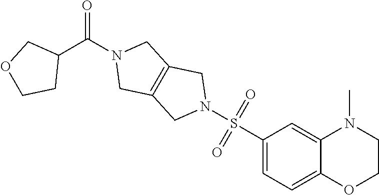 [SMILES: CN1CCOc2ccc(S(=O)(=O)N3CC4=C(CN(C(=O)C5CCOC5)C4)C3)cc21] | (5-((4-methyl-3,4-dihydro-2H- benzo[b][1,4]oxazin-6-yl)sulfonyl)- 3,4,5,6-tetrahydropyrrolo[3,4-c]pyrrol- 2(1H)-yl)(tetrahydrofuran-3- yl)methanone |
| 48 | 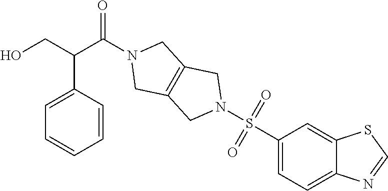 [SMILES: O=C(C(CO)c1ccccc1)N1CC2=C(C1)CN(S(=O)(=O)c1ccc3ncsc3c1)C2] | 1-(5-(benzo[d]thiazol-6-ylsulfonyl)- 3,4,5,6-tetrahydropyrrolo[3,4-c]pyrrol- 2(1H)-yl)-3-hydroxy-2-phenylpropan-1- one |
| 49 | 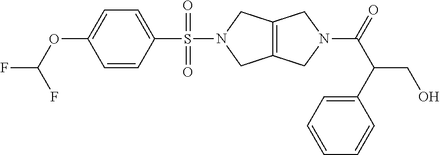 [SMILES: O=C(C(CO)c1ccccc1)N1CC2=C(C1)CN(S(=O)(=O)c1ccc(OC(F)F)cc1)C2] | 1-(5-((4- (difluoromethoxy)phenyl)sulfonyl)- 3,4,5,6-tetrahydropyrrolo[3,4-c]pyrrol- 2(1H)-yl)-3-hydroxy-2-phenylpropan-1- one |
| 50 | 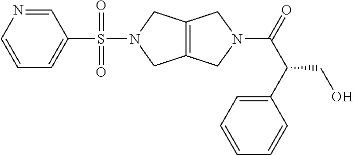 [SMILES: O=C([C@H](CO)c1ccccc1)N1CC2=C(C1)CN(S(=O)(=O)c1cccnc1)C2] | (2S)-3-hydroxy-2-phenyl-1-[5-(pyridine- 3-sulfonyl)-1H,2H,3H,4H,5H,6H- pyrrolo[3,4-c]pyrrol-2-yl]propan-1-one |
| 51 | 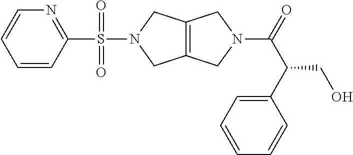 [SMILES: O=C([C@H](CO)c1ccccc1)N1CC2=C(C1)CN(S(=O)(=O)c1ccccn1)C2] | (2S)-3-hydroxy-2-phenyl-1-[5-(pyridine- 2-sulfonyl)-1H,2H,3H,4H,5H,6H- pyrrolo[3,4-c]pyrrol-2-yl]propan-1-one |
| 52 | 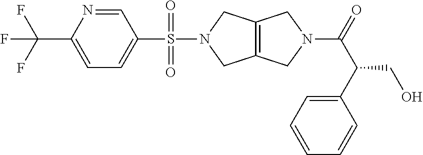 [SMILES: O=C([C@H](CO)c1ccccc1)N1CC2=C(C1)CN(S(=O)(=O)c1ccc(C(F)(F)F)nc1)C2] | (2S)-3-hydroxy-2-phenyl-1-(5-{[6- (trifluoromethyl)pyridin-3-yl]sulfonyl}- 1H,2H,3H,4H,5H,6H-pyrrolo[3,4- c]pyrrol-2-yl)propan-1-one |
| 53 | 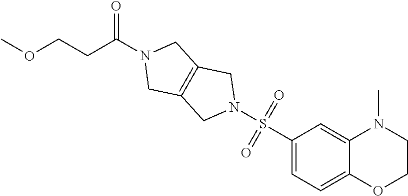 [SMILES: COCCC(=O)N1CC2=C(C1)CN(S(=O)(=O)c1ccc3c(c1)N(C)CCO3)C2] | 3-methoxy-1-(5-((4-methyl-3,4-dihydro- 2H-benzo[b][1,4]oxazin-6-yl)sulfonyl)- 3,4,5,6-tetrahydropyrrolo[3,4-c]pyrrol- 2(1H)-yl)propan-1-one; and |
| 54 | 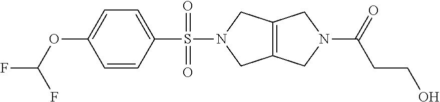 [SMILES: O=C(CCO)N1CC2=C(C1)CN(S(=O)(=O)c1ccc(OC(F)F)cc1)C2] | 1-(5-((4- (difluoromethoxy)phenyl)sulfonyl)- 3,4,5,6-tetrahydropyrrolo[3,4-c]pyrrol- 2(1H)-yl)-3-hydroxypropan-1-one. |
| 55 | 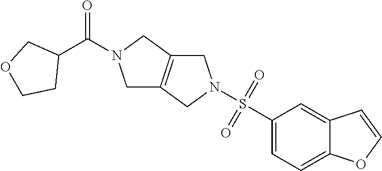 [SMILES: O=C(C1CCOC1)N1CC2=C(C1)CN(S(=O)(=O)c1ccc3occc3c1)C2] | (5-(benzofuran-5-ylsulfonyl)-3,4,5,6- tetrahydropyrrolo[3,4-c]pyrrol-2(1H)- yl)(tetrahydrofuran-3-yl)methanone |
| 56 | 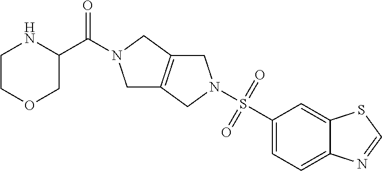 [SMILES: O=C(C1COCCN1)N1CC2=C(C1)CN(S(=O)(=O)c1ccc3ncsc3c1)C2] | (5-(benzo[d]thiazol-6-ylsulfonyl)- 3,4,5,6-tetrahydropyrrolo[3,4-c]pyrrol- 2(1H)-yl)(morpholin-3-yl)methanone |
| 57 | 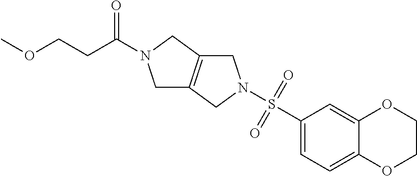 [SMILES: COCCC(=O)N1CC2=C(C1)CN(S(=O)(=O)c1ccc3c(c1)OCCO3)C2] | 1-(5-((2,3-dihydrobenzo[b][1,4]dioxin- 6-yl)sulfonyl)-3,4,5,6- tetrahydropyrrolo[3,4-c]pyrrol-2(1H)- yl)-3-methoxypropan-1-one |
| 58 | 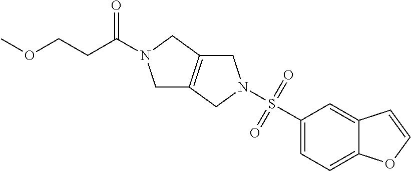 [SMILES: COCCC(=O)N1CC2=C(C1)CN(S(=O)(=O)c1ccc3occc3c1)C2] | 1-(5-(benzofuran-5-ylsulfonyl)-3,4,5,6- tetrahydropyrrolo[3,4-c]pyrrol-2(1H)- yl)-3-methoxypropan-1-one |

62. A pharmaceutical composition comprising a compound of any one of embodiments 1-61 and a pharmaceutically acceptable carrier.
63. A method of treating a disease or disorder associated with modulation of pyruvate kinase (PKR), comprising administering to a patient in need thereof an effective amount of a compound of any one of embodiments 1-61 or a composition of embodiment 62.
64. A method of treating a disease associated with decreased activity of PKR in a subject in need thereof, comprising administering to the subject an effective amount of a compound of any one of embodiments 1-61 or a composition of embodiment 62.
65. A method of activating PKR, comprising administering to a subject in need thereof an effective amount of a compound of any one of embodiments 1-61 or a composition of embodiment 62.
66. A method of increasing the lifetime of red blood cells comprising administering to a subject in need thereof an effective amount of a compound of any one of embodiments 1-61 or a composition of embodiment 62.
67. A method of regulating 2,3-diphosphoglycerate levels in blood comprising administering to a subject in need thereof an effective amount of a compound of any one of embodiments 1-61 or a composition of embodiment 62.
68. A method of regulating ATP levels in blood comprising administering to a subject in need thereof an effective amount of a compound of any one of embodiments 1-61 or a composition of embodiment 62.
69. A method of treating hereditary non-spherocytic hemolytic anemia comprising administering to a subject in need thereof an effective amount of a compound of any one of embodiments 1-61 or a composition of embodiment 62.
70. A method of treating a disease or disorder associated with increased 2,3-diphosphoglycerate levels comprising administering to a subject in need thereof an effective amount of a compound of any one of embodiments 1-61 or a composition of embodiment 62.
71. A method of a treating disease or disorder associated with decreased ATP levels comprising administering to a subject in need thereof an effective amount of a compound of any one of embodiments 1-61 or a composition of embodiment 62.
72. The method of any of embodiments 63-64 or 70-71, wherein the disease or disorder is selected from the group consisting of sickle cell disease, sickle cell anemia, thalassemia (e.g., beta-thalassemia), hereditary non-spherocytic hemolytic anemia, hemolytic anemia (e.g., chronic hemolytic anemia caused by phosphoglycerate kinase deficiency), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), and anemia of chronic diseases.
73. A method of treating a disease or disorder comprising administering to a patient in need thereof an effective amount of a compound of any one of embodiments 1-61 or a composition of embodiment 62.
74. The method of embodiment 73, wherein the disease or disorder is selected from the group consisting of sickle cell disease, sickle cell anemia, thalassemia (e.g., beta-thalassemia), hereditary non-spherocytic hemolytic anemia, hemolytic anemia (e.g., chromic hemolytic anemia caused by phosphoglycerate kinase deficiency), hereditary spherocytosis, hereditary elliptocytosis, abetalipoproteinemia (or Bassen-Kornzweig syndrome), paroxysmal nocturnal hemoglobinuria, acquired hemolytic anemia (e.g., congenital anemias (e.g., enzymopathies)), and anemia of chronic diseases.
75. The method of embodiment 74, wherein the disease or disorder is sickle cell anemia.
76. The method of embodiment 74, wherein the disease or disorder is hemolytic anemia.
77. The method of embodiment 74, wherein the disease or disorder is beta thalassemia.
78. A PKR Activating Compound having a % Fold@1.54 μM of at least 75%, according to the Luminescence Assay Protocol of Example 47.
79. The PKR Activating Compound of embodiment 78 having a % Fold@1.54 μM of 75-500%,
80. The PKR Activating Compound of embodiment 79 having a % Fold@1.54 μM of 250-500%.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims

The following are illustrative, but non-limiting, examples of certain embodiments of the present disclosure. The synthetic schemes are presented for the synthesis of certain compounds herein disclosed.

Definitions used in the following Schemes and elsewhere herein are:
ACN acetonitrile
AcOH acetic acid
AIBN azobisisobutyronitrile
AlCl₃ trichloroaluminum
Boc₂O di-tert-butyl dicarbonate
NaBH₄ sodium borohydride
BOP ammonium 4-(3-(pyridin-3-ylmethyl)ureido)benzenesulfinate
Brine saturated aqueous sodium chloride solution
CDCl₃ deuterated chloroform
δ chemical shift
DCM dichloromethane or methylene chloride
DCE dichloroethane
DIEA N,N-diisopropylethylamine
DMA N,N-dimethylacetamide
DMF N,N-dimethylformamide
DMSO dimethylsulfoxide
DMT dimercaptotriazine
EDCI N₁-((ethylimino)methylene)-N₃,N₃-dimethylpropane-1,3-diamine hydrochloride
equiv equivalents
EtOAc, EA ethyl acetate
EtOH ethanol
h hour
HCl hydrochloric acid
¹H NMR proton nuclear magnetic resonance
HOAc acetic acid
HATU 2-(3H-[1,2,3]triazolo[4,5-b]pyridin-3-yl)-1,1,3,3-tetramethylisouronium hexafluorophosphate
HBTU O-(Benzotriazol-yl)N,N,N′,N′-tetramethyluronium hexafluorophosphate
HOBT 1H-benzo[d][1,2,3]triazol-1-ol hydrate
HPLC high performance liquid chromatography
Hz hertz
KOAc potassium acetate
LCMS liquid chromatography/mass spectrometry
LDA lithium diisopropylamide
(M+1) mass+1
m-CPBA m-chloroperbenzoic acid
MeOH methanol
min minute(s)
n-BuLi n-butyl lithium
NCS N-chlorosuccinimide
NaH sodium hydride
NaHCO₃ sodium bicarbonate
NaOH sodium hydroxide
Na₂SO₄ sodium sulfate
Pd₂(dba)₃ tris(dibenzylideneacetone)dipalladium(0)
PFA paraformaldehyde
PTLC preparative thin layer chromatography
RT room temperature
Rt retention time
SFC supercritical fluid chromatography
SPE solid phase extraction
TEA triethylamine
TFAA trifluoroacetic anhydride
TMSCN trimethylsilyl cyanide
THF tetrahydrofuran
TLC thin layer chromatography
Xantphos 4,5-Bis(diphenylphosphino)-9,9-dimethylxanthene
ZnI₂ zinc iodide

### Materials

Unless otherwise noted, all materials were obtained from commercial suppliers and were used without further purification. Anhydrous solvents were obtained from Sigma-Aldrich (Milwaukee, WI) and used directly. All reactions involving air- or moisture-sensitive reagents were performed under a nitrogen atmosphere.

Intermediate 1: 2-[2H,3H-[1,4]dioxino[2,3-b]pyridine-7-sulfonyl]-1H,2H,3H,4H,5H,6H-pyrrolo[3,4-c]pyrrole

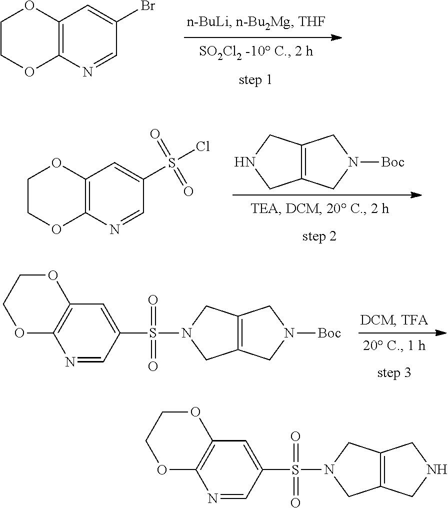

### Step 1. 2H,3H-[1,4]dioxino[2,3-b]pyridine-7-sulfonyl chloride

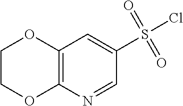 [SMILES: O=S(=O)(Cl)c1cnc2c(c1)OCCO2]

Into a 100 mL round-bottom flask purged and maintained with an inert atmosphere of nitrogen was placed a solution of n-BuLi in hexane (2.5 M, 2 mL, 5.0 mmol, 0.54 equiv) and a solution of n-Bu₂Mg in heptanes (1.0 M, 4.8 mL, 4.8 mmol, 0.53 equiv). The resulting solution was stirred for 10 min at RT (20° C.). This was followed by the dropwise addition of a solution of 7-bromo-2H,3H-[1,4]dioxino[2,3-b]pyridine (2 g, 9.26 mmol, 1.00 equiv) in tetrahydrofuran (16 mL) with stirring at −10° C. in 10 min. The resulting mixture was stirred for 1 h at −10° C. The reaction mixture was slowly added to a solution of thionyl chloride (16 mL) at −10° C. The resulting mixture was stirred for 0.5 h at −10° C. The reaction was then quenched by the careful addition of 30 mL of saturated ammonium chloride solution at 0° C. The resulting mixture was extracted with 3×50 mL of dichloromethane. The organic layers were combined, dried over anhydrous sodium sulfate, filtered and concentrated under vacuum. The residue was purified by silica gel column chromatography, eluting with ethyl acetate/petroleum ether (1:3). This provided 1.3 g (60%) of 2H,3H-[1,4]dioxino[2,3-b]pyridine-7-sulfonyl chloride as a white solid. LCMS m/z: calculated for C₇H₆C₁NO₄S: 235.64; found: 236 [M+H]⁺.

### Step 2. tert-Butyl 5-[2H,3H-[1,4]dioxin[2,3-b]pyridine-7-sulfonyl]-1H,2H,3H,4H,5H,6H-pyrrolo[3,4-c]pyrrole-2-carboxylate

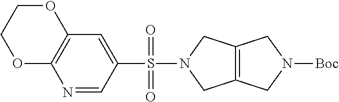 [SMILES: CC(C)(C)OC(=O)N1CC2=C(C1)CN(S(=O)(=O)c1cnc3c(c1)OCCO3)C2]

Into a 100-mL round-bottom flask was placed 2H,3H-[1,4]dioxino[2,3-b]pyridine-7-sulfonyl chloride (1.3 g, 5.52 mmol, 1.00 equiv), tert-butyl 1H,2H,3H,4H,5H,6H-pyrrolo[3,4-c]pyrrole-2-carboxylate (1.16 g, 5.52 mmol), dichloromethane (40 mL), and triethylamine (1.39 g, 13.74 mmol, 2.49 equiv). The solution was stirred for 2 h at 20° C., then diluted with 40 mL of water. The resulting mixture was extracted with 3×30 mL of dichloromethane. The organic layers were combined, dried over anhydrous sodium sulfate, filtered and concentrated under vacuum. The residue was purified by silica gel column chromatography, eluting with dichloromethane/methanol (10:1). This provided 1.2 g (53%) of tert-butyl 5-[2H,3H-[1,4]dioxino[2,3-b]pyridine-7-sulfonyl]-1H,2H,3H,4H,5H,6H-pyrrolo[3,4-c]pyrrole-2-carboxylate as a yellow solid. LCMS m/z: calculated for C₁₈H₂₃N₃O₆S: 409.46; found: 410 [M+H]⁺.

### Step 3. 2-[2H,3H-[1,4]dioxino[2,3-b]pyridine-7-sulfonyl]-1H,2H,3H,4H,5H,6H-pyrrolo[3,4-c]pyrrole

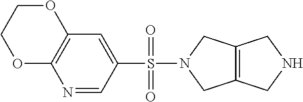 [SMILES: O=S(=O)(c1cnc2c(c1)OCCO2)N1CC2=C(CNC2)C1]

Into a 100-mL round-bottom flask was placed tert-butyl 5-[2H,3H-[1,4]dioxino[2,3-b]pyridine-7-sulfonyl]-1H,2H,3H,4H,5H,6H-pyrrolo[3,4-c]pyrrole-2-carboxylate (1.2 g, 2.93 mmol, 1.00 equiv), dichloromethane (30 mL), and trifluoroacetic acid (6 mL). The solution was stirred for 1 h at 20° C. The resulting mixture was concentrated under vacuum. The residue was dissolved in 10 ml of methanol and the pH was adjusted to 8 with sodium bicarbonate (2 mol/L). The resulting solution was extracted with 3×10 mL of dichloromethane. The organic layers were combined, dried over anhydrous sodium sulfate, filtered and concentrated under vacuum. The crude product was purified by silica gel column chromatography, eluting with dichloromethane/methanol (10:1). This provided 650 mg (72%) of 2-[2H,3H-[1,4]dioxino[2,3-b]pyridine-7-sulfonyl]-1H,2H,3H,4H,5H,6H-pyrrolo[3,4-c]pyrrole as a yellow solid. LCMS m/z: calculated for C₁₃H₁₅N₃O₄S: 309.34; found: 310 [M+H]⁺.

### Intermediate 2: 2-((2,3-dihydrobenzo[b][1,4]dioxin-6-yl)sulfonyl)-1,2,3,4,5,6-hexahydropyrrolo[3,4-c]pyrrole

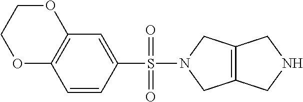 [SMILES: O=S(=O)(c1ccc2c(c1)OCCO2)N1CC2=C(CNC2)C1]

Prepared as described for Intermediate 1 (step 2 and step 3), using the appropriate synthetic precursors.

### Step 2: tert-butyl 5-(2,3-dihydro-1,4-benzodioxine-6-sulfonyl)-1H,2H,3H,4H,5H,6H-pyrrolo[3,4-c]pyrrole-2-carboxylate

This resulted in 170 mg (98%) of tert-butyl 5-(2,3-dihydro-1,4-benzodioxine-6-sulfonyl)-1H,2H,3H,4H,5H,6H-pyrrolo[3,4-c]pyrrole-2-carboxylate as a brown solid. LCMS: m/z=409 [M+H]⁺.

### Step 3: 2-(2,3-dihydro-1,4-benzodioxine-6-sulfonyl)-1H,2H,3H,4H,5H,6H-pyrrolo[3,4-c]pyrrole

This resulted in 200 mg (91%) of 2-(2,3-dihydro-1,4-benzodioxine-6-sulfonyl)-1H,2H,3H,4H,5H,6H-pyrrolo[3,4-c]pyrrole as brown oil. LCMS: m/z=309 [M+H]⁺.

Intermediates 3 and 4. (S)-3-hydroxy-2-phenylpropanoic acid and (R)-3-hydroxy-2-phenylpropanoic acid

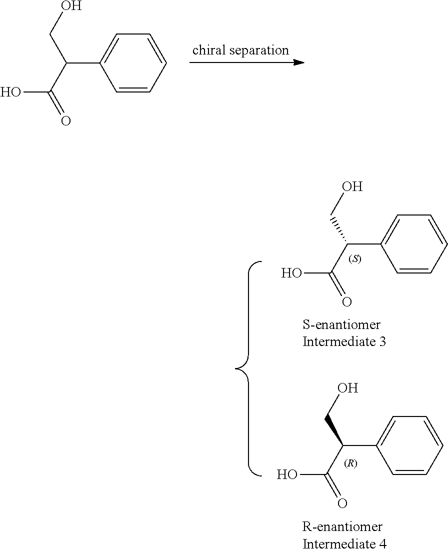

Materials 3-Hydroxy-2-phenylpropanoic acid (1 g) was separated by Prep-SFC with the following conditions: Instrument Name: SHIMADZU LC-20AD, LC parameters: Pump Mode: Binary gradient, Start Conc. of Pump B: 100.0%, Total Flow: 170 mL/min, Phase A, Phase B: MeOH (0.1% HAC), Column Name: CHIRALPAK AD-H, Length: 100 mm, Internal Diameter: 4.6 mm, Particle Size: 5 μm, Column Temp: 20° C., PDA Model: SPD-M20A, Wavelength: from 190 nm to 500 nm. This provided peak 1: (Rt=5.76 min) 380 mg of (S)-3-hydroxy-2-phenylpropanoic acid as a white solid, and peak 2: (Rt=6.87 min) 370 mg of (R)-3-hydroxy-2-phenylpropanoic acid as a white solid.

¹H NMR (300 MHz, DMSO-d₆): δ ppm 12.31 (br s, 1H), 7.40-7.20 (m, 5H), 4.94 (br s, 1H), 3.92 (t, J=9 Hz, 1H), 3.67-3.54 (m, 2H). S-enantiomer: α_{D}^{16.7}=−110 (C 0.02, water); [literature: −79] R-enantiomer: α_{D}^{16.7}=+125 (C 0.02, water).

### Intermediate 5: 1-(5-((2,3-dihydro-[1,4]dioxino[2,3-b]pyridin-7-yl)sulfonyl)-3,4,5,6-tetrahydropyrrolo[3,4-c]pyrrol-2(1H)-yl)-2-(pyridin-2-yl)ethan-1-one

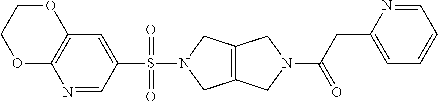 [SMILES: O=C(Cc1ccccn1)N1CC2=C(C1)CN(S(=O)(=O)c1cnc3c(c1)OCCO3)C2]

Prepared according to the reaction conditions described for Example 1 from the appropriate reagents. 1-(5-((2,3-Dihydro-[1,4]dioxino[2,3-b]pyridin-7-yl)sulfonyl)-3,4,5,6-tetrahydropyrrolo[3,4-c]pyrrol-2(1H)-yl)-2-(pyridin-2-yl)ethan-1-one was isolated as a white solid (300 mg, 76%). LC-MS: m/z: calculated for C₂₀H₂₀N₄O₅S: 428.12; found 429.10 [M+H]⁺.

### Intermediate 6: 1-(5-((4-(difluoromethoxy)phenyl)sulfonyl)-3,4,5,6-tetrahydropyrrolo[3,4-c]pyrrol-2(1H)-yl)-2-(pyridin-2-yl)ethan-1-one

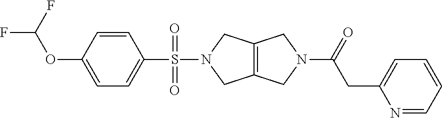 [SMILES: O=C(Cc1ccccn1)N1CC2=C(C1)CN(S(=O)(=O)c1ccc(OC(F)F)cc1)C2]

Prepared according to the reaction conditions described for Example 7 from the appropriate reagents. The crude material was purified by prep-HPLC: Column: SunFire Prep C_{18 5} μm 19*150 mm; mobile phase: water (contains 0.1% TFA) and CH₃CN with a gradient of 43% to 73% CH₃CN in 7 min; detector UV wavelength: 220 nm. This resulted in 25.6 mg (21%) of 1-(5-((4-(difluoromethoxy)phenyl)sulfonyl)-3,4,5,6-tetrahydropyrrolo[3,4-c]pyrrol-2(1H)-yl)-2-(pyridin-2-yl)ethan-1-one as a white solid.

¹H NMR (300 MHz, DMSO-d₆, ppm): δ8.50-8.51 (d, J=4.2 Hz, 1H), 7.84-7.89 (m, 2H), 7.71-7.76 (t, J=7.5 Hz, 1H), 7.44-7.46 (d, J=8.1 Hz, 1H), 7.23-7.29 (m, 3H), 6.37-6.85 (t, J=72.6 Hz, 1H), 4.40 (br, 2H), 3.97-4.14 (br, 6H), 3.90-3.94 (br, 2H). LC-MS m/z: Calculated for C₂₀H₁₉F₂N₃O₄S: 435.11; found: 436 [M+H]⁺.

### Intermediate 7: 2-(Benzofuran-5-ylsulfonyl)-1,2,3,4,5,6-hexahydropyrrolo[3,4-c]pyrrole hydrochloride

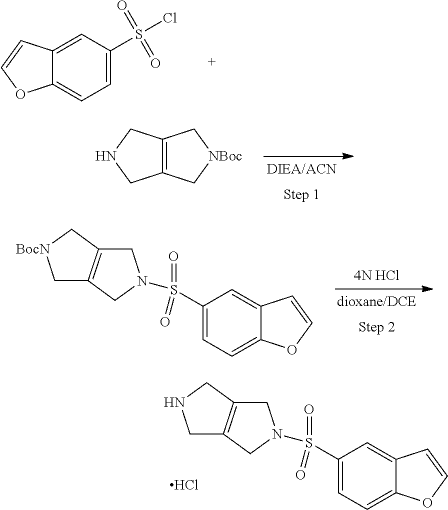

### Step 1. tert-Butyl 5-(benzofuran-5-ylsulfonyl)-3,4,5,6-tetrahydropyrrolo[3,4-c]pyrrole-2(1H)-carboxylate

To a solution of tert-butyl 3,4,5,6-tetrahydropyrrolo[3,4-c]pyrrole-2(1H)-carboxylate (0.7 g, 3.33 mmol) in acetonitrile (20 mL) and DIEA (1.70 mL, 9.76 mmol) was added benzofuran-5-sulfonyl chloride (17.48 ml, 3.50 mmol) in 1,4 dioxane (17 mL). The resulting mixture was stirred at RT overnight. The reaction mixture was worked up with saturated ammonium chloride solution and EtOAc. The combined organics were washed with brine, dried over Na₂SO₄, filtered, and concentrated under reduced pressure to provide tert-butyl 5-(benzofuran-5-ylsulfonyl)-3,4,5,6-tetrahydropyrrolo[3,4-c]pyrrole-2(1H)-carboxylate (1.3 g, 3.33 mmol, 100%) as an oil. LCMS: m/z=413 [M+Na]⁺.

### Step 2. 2-(Benzofuran-5-ylsulfonyl)-1,2,3,4,5,6-hexahydropyrrolo[3,4-c]pyrrole hydrochloride

Tert-butyl 5-(benzofuran-5-ylsulfonyl)-3,4,5,6-tetrahydropyrrolo[3,4-c]pyrrole-2(1H)-carboxylate (1.3 g, 3.33 mmol) was dissolved in a mixture of methanol (3.0 mL), DCE (10.0 mL) and 4 M HCl in 1,4-dioxane (5.0 mL). The reaction was heated at 50° C. for 2 h. The solvents were evaporated under reduced pressure and the reaction mixture was azeotropically dried with toluene and dried further under vacuum overnight to provide 2-(benzofuran-5-ylsulfonyl)-1,2,3,4,5,6-hexahydropyrrolo[3,4-c]pyrrole hydrochloride (0.95 mg, 3.33 mmol, 100%). LCMS: m/z=291 [M+H]⁺.

### Intermediate 8: 1-(6-((3,4,5,6-Tetrahydropyrrolo[3,4-c]pyrrol-2(1H)-yl)sulfonyl)-2,3-dihydro-4H-benzo[b][1,4]oxazin-4-yl)ethan-1-one hydrochloride

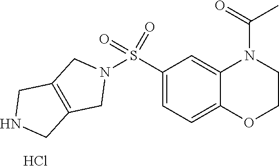 [SMILES: CC(=O)N1CCOc2ccc(S(=O)(=O)N3CC4=C(CNC4)C3)cc21 ; Cl]

Prepared as described for Intermediate 7, using the appropriate synthetic precursors. 94% overall yield. LCMS: m/z=350 [M+H]⁺.

### Intermediate 9: 2-(Pyridin-2-ylsulfonyl)-1,2,3,4,5,6-hexahydropyrrolo[3,4-c]pyrrole, hydrochloride salt

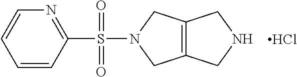 [SMILES: O=S(=O)(c1ccccn1)N1CC2=C(CNC2)C1 ; Cl]

Prepared as described for Intermediate 7, using the appropriate synthetic precursors.

### Step 1. tert-Butyl 5-(pyridine-2-sulfonyl)-1H,2H,3H,4H,5H,6H-pyrrolo[3,4-c]pyrrole-2-carboxylate

Isolated as a orange solid (570 mg, 36%). The material was used without further purification. LCMS: m/z=352 [M+H]⁺.

### Step 2. 2-(Pyridin-2-ylsulfonyl)-1,2,3,4,5,6-hexahydropyrrolo[3,4-c]pyrrole, hydrochloride salt

Isolated as a white solid (467 mg, quantitative yield). LCMS: m/z=252 [M+H]⁺.

### Intermediate 10: 3-Hydroxy-2,2-dimethyl-1-(3,4,5,6-tetrahydropyrrolo[3,4-c]pyrrol-2(1H)-yl)propan-1-one hydrochloride

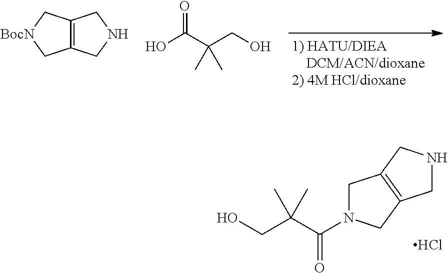

To a solution of tert-butyl 3,4,5,6-tetrahydropyrrolo[3,4-c]pyrrole-2(1H)-carboxylate (50.5 mg, 0.24 mmol) in DCM (1.2 mL) and DIEA (105 μL, 0.60 mmol) was added 3-hydroxy-2,2-dimethylpropanoic acid (28.4 mg, 0.240 mmol) in 1,4 dioxane (1.2 mL), followed by a solution of HATU (630 μl, 0.252 mmol) in acetonitrile (1.3 mL). The reaction mixture was stirred at RT for 3 hours and worked-up with 1 N NaOH (aqueous) and EtOAc. The resulting material was dissolved in DCM (0.9 mL) and 4 M HCl in 1,4-dioxane (0.36 mL) was added. The mixture was stirred at RT overnight. The reaction was concentrated, azeotropically dried with toluene and dried further under vacuum to give 3-hydroxy-2,2-dimethyl-1-(3,4,5,6-tetrahydropyrrolo[3,4-c]pyrrol-2(1H)-yl)propan-1-one hydrochloride (44.4 mg, 0.180 mmol, 75.0% yield).

Intermediate 11: (R)-(tetrahydrofuran-3-yl)(3,4,5,6-tetrahydropyrrolo[3,4-c]pyrrol-2(1H)-yl)methanone hydrochloride salt

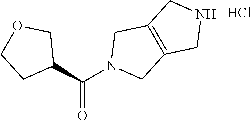

Prepared according to the procedure for Intermediate 10, using the appropriate synthetic precursors

### Intermediate 12: 6-((3,4,5,6-Tetrahydropyrrolo[3,4-c]pyrrol-2(1H)-yl)sulfonyl)benzo[d]thiazole hydrochloride

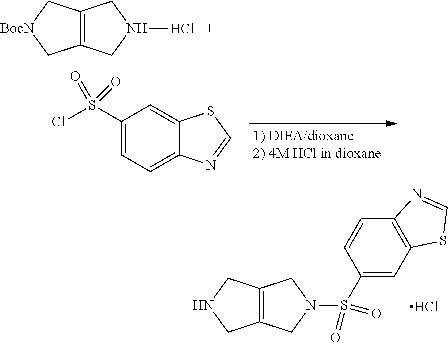

To a 50 mL round-bottomed flask was added tert-butyl 3,4,5,6-tetrahydropyrrolo[3,4-c]pyrrole-2(1H)-carboxylate hydrochloride (0.5 g, 2.026 mmol), DIEA (1.059 ml, 6.08 mmol), and dioxane (10 mL) to give a brown suspension. Benzo[d]thiazole-6-sulfonyl chloride (0.497 g, 2.128 mmol) was added. The reaction was heated at 50° C. with stirring for 2 hours. The volatiles were removed under reduced pressure. The residue was resuspended in dioxane (10 mL) and 4 M HCl in dioxane (5.07 ml, 20.26 mmol) was added. The reaction was heated at 50° C. with stirring for 2 hours. The volatiles were removed under reduced pressure to give 6-((3,4,5,6-tetrahydropyrrolo[3,4-c]pyrrol-2(1H)-yl)sulfonyl)benzo[d]thiazole hydrochloride (0.640 g, 1.865 mmol, 92%) as a brown oil that was used without further purification. LCMS: m/z=307.9 [M+H]⁺.

### Intermediate 13: 2-[[4-(difluoromethoxy)benzene]sulfonyl]-1H,2H,3H,4H,5H,6H-pyrrolo[3,4-c]pyrrole hydrochloride

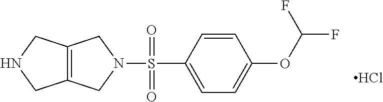 [SMILES: O=S(=O)(c1ccc(OC(F)F)cc1)N1CC2=C(CNC2)C1 ; Cl]

Prepared according to the procedure for Intermediate 12, using the appropriate synthetic precursors. Obtained 0.652 g (1.848 mmol, 91%). LCMS: m/z=317.1 [M+H]⁺.

Intermediate 14: 2-((6-(trifluoromethyl)pyridin-3-yl)sulfonyl)-1,2,3,4,5,6-hexahydropyrrolo[3,4-c]pyrrole HCl salt

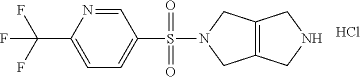 [SMILES: O=S(=O)(c1ccc(C(F)(F)F)nc1)N1CC2=C(CNC2)C1 ; Cl]

Prepared according to the procedure for Intermediate 12, using the appropriate synthetic precursors. LCMS: m/z=319.9 [M+H]⁺.

### Intermediate 15: 2,2-Dimethyl-6-[1H,2H,3H,4H,5H,6H-pyrrolo[3,4-c]pyrrole-2-sulfonyl]-3,4-dihydro-2H-1,4-benzoxazine, TFA salt

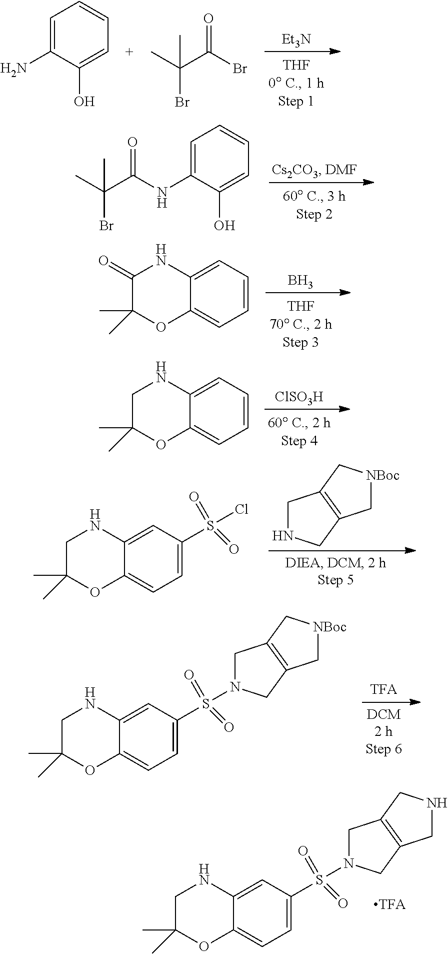

### Step 1. 2-Bromo-N-(2-hydroxyphenyl)-2-methylpropanamide

To a 500-mL 3-necked round-bottom flask was added a mixture of 2-aminophenol (5 g, 45.82 mmol, 1.00 equiv), THF (150 mL) and TEA (5.1 g, 50.40 mmol). 2-Bromo-2-methylpropanoyl bromide (11.6 g, 50.46 mmol, 1.10 equiv) was then added dropwise. The solution was stirred for 1 h at 0° C. The reaction was then quenched by the addition of water (15 mL). The solution was extracted with ethyl acetate (3×200 mL), then the extract was washed with brine (2×150 mL) and dried over anhydrous sodium sulfate to provide 2-bromo-N-(2-hydroxyphenyl)-2-methylpropanamide (11.0 g, 93%) as a yellow oil. LCMS: m/z=259 [M+H]⁺.

### Step 2. 2,2-Dimethyl-3,4-dihydro-2H-1,4-benzoxazin-3-one

To a 250-mL 3-necked round-bottom flask was added 2-bromo-N-(2-hydroxyphenyl)-2-methylpropanamide (6 g, 23.25 mmol, 1.00 equiv), Cs₂CO₃ (9.85 g, 30.23 mmol, 1.30 equiv), and DMF (180 mL). The reaction mixture was stirred for 3 h at 60° C., then quenched by the addition of water (200 mL). The mixture was extracted with ethyl acetate (3×200 mL), and the extract was washed with brine (2×150 mL) and dried over anhydrous sodium sulfate to provide 2,2-dimethyl-3,4-dihydro-2H-1,4-benzoxazin-3-one (2.2 g, 53%) as a white solid. LCMS: m/z=178 [M+H]⁺.

### Step 3. 2,2-Dimethyl-3,4-dihydro-2H-1,4-benzoxazine

A mixture of 2,2-dimethyl-3,4-dihydro-2H-1,4-benzoxazin-3-one (2.76 g, 15.58 mmol, 1.00 equiv) and THF (10 mL) was prepared in a 100 mL 3-necked round-bottom flask. The mixture was cooled to 0° C., then BH₃·THF (1 M, 23.4 mL, 1.50 equiv) was added dropwise with stirring. The reaction mixture was stirred for 2 h at 70° C. The reaction was quenched by addition of methanol (4 mL), then concentrated under vacuum. The pH was adjusted to 6.0 with 1 N HCl aqueous solution and stirred for 30 minutes at RT. It was then neutralized with saturated aqueous sodium carbonate solution and the pH was adjusted to 8.0. The solution was extracted with ethyl acetate (50 mL), then the organic phase washed with brine (30 mL), dried over anhydrous sodium sulfate, filtered and concentrated under vacuum to provide 2,2-dimethyl-3,4-dihydro-2H-1,4-benzoxazine (2.90 g) as a colorless oil. The material was used without further purification. LCMS: m/z=164 [M+H]⁺.

### Step 4. 2,2-Dimethyl-3,4-dihydro-2H-1,4-benzoxazine-6-sulfonyl chloride

2,2-Dimethyl-3,4-dihydro-2H-1,4-benzoxazine (500 mg, 3.06 mmol, 1.00 equiv) was placed in a 100-mL 3-necked round-bottom flask and cooled to 0° C. Sulfurochloridic acid (5 g, 42.91 mmol, 14.01 equiv) was added dropwise. The solution was stirred for 2 h at 60° C. The reaction was then quenched by the addition of water (50 mL) and extracted with dichloromethane (50 mL). The organic phase was washed with brine (3×20 mL), dried over anhydrous sodium sulfate and concentrated under vacuum to provide 2,2-dimethyl-3,4-dihydro-2H-1,4-benzoxazine-6-sulfonyl chloride (0.14 g, 17%) as a yellow oil.

### Step 5. tert-Butyl 5-(2,2-dimethyl-3,4-dihydro-2H-1,4-benzoxazine-6-sulfonyl)-1H,2H,3H,4H,5H,6H-pyrrolo[3,4-c]pyrrole-2-carboxylate

To a 25-mL round-bottom flask was added 2,2-dimethyl-3,4-dihydro-2H-1,4-benzoxazine-6-sulfonyl chloride (124 mg, 0.47 mmol, 1.00 equiv) and dichloromethane (2 mL), followed by addition of tert-butyl 1H,2H,3H,4H,5H,6H-pyrrolo[3,4-c]pyrrole-2-carboxylate (100 mg, 0.48 mmol, 1.00 equiv) and DIEA (110 mg, 0.85 mmol, 2.00 equiv). The solution was stirred for 2 h at 25° C. The reaction was then quenched by the addition of ethyl acetate (20 mL). The mixture was washed with brine (3×10 mL), dried over anhydrous sodium sulfate, filtered, and concentrated under vacuum. The residue was purified by flash chromatography on silica gel with dichloromethane/ethyl acetate (10:1) to provide tert-butyl 5-(2,2-dimethyl-3,4-dihydro-2H-1,4-benzoxazine-6-sulfonyl)-1H,2H,3H,4H,5H,6H-pyrrolo[3,4-c]pyrrole-2-carboxylate (0.102 g, 49%) as a light yellow oil. LCMS: m/z=436 [M+H]⁺.

### Step 6. 2,2-Dimethyl-6-[1H,2H,3H,4H,5H,6H-pyrrolo[3,4-c]pyrrole-2-sulfonyl]-3,4-dihydro-2H-1,4-benzoxazine, TFA salt

To a solution of tert-butyl 5-(2,2-dimethyl-3,4-dihydro-2H-1,4-benzoxazine-6-sulfonyl)-1H,2H,3H,4H,5H,6H-pyrrolo[3,4-c]pyrrole-2-carboxylate (102 mg, 0.23 mmol, 1.00 equiv) in dichloromethane (3 mL) was added TFA (600 mg, 5.31 mmol, 23.00 equiv). The solution was stirred for 2 h at 25° C. under an atmosphere of nitrogen. The reaction mixture was concentrated under vacuum to provide 2,2-dimethyl-6-[1H,2H,3H,4H,5H,6H-pyrrolo[3,4-c]pyrrole-2-sulfonyl]-3,4-dihydro-2H-1,4-benzoxazine, TFA salt (85 mg) as a light yellow oil. The material was used without further purification. LCMS: m/z=336 [M+H]⁺.

### Intermediates 17 and 18: (2R,3S and 2S,3R) 3-hydroxy-2-phenylbutanoic acid and (2S,3S and 2R,3R) 3-hydroxy-2-phenylbutanoic acid

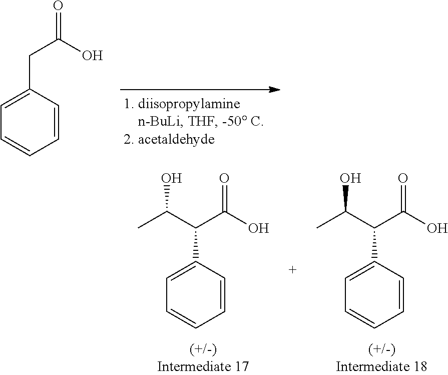

To a 250-mL 3-necked round-bottom flask purged and maintained with an inert atmosphere of nitrogen were added 2-phenylacetic acid (2 g, 14.69 mmol, 1.00 equiv) and tetrahydrofuran (50 mL). LDA (3.00 equiv, 22 mL, 2 N in THF) was added with stirring at −50° C. The reaction mixture was stirred for 1 h at −50° C., then acetaldehyde (1.94 g, 3.00 equiv) was added. The reaction was stirred for 1 h at −50° C. and then 1 h at RT. 3 N Aqueous hydrogen chloride solution (3 N, 20 mL) was added and the mixture was extracted with ethyl acetate (2×100 mL). The organic phase was dried over anhydrous sodium sulfate, filtered and concentrated under vacuum. The residue was purified by a flash column chromatography on silica gel eluted with dichloromethane/methanol (10:1). This provided:

Intermediate 17: A mixture of (2R,3S and 2S,3R)-3-hydroxy-2-phenylbutanoic acid (stereochemical configuration assumed). Obtained 700 mg (3.89 mmol, 26%) as an oil. LCMS: m/z=222 [M+1].

Intermediate 18: A mixture of (2R,3R and 2S,3S)-3-hydroxy-2-phenylbutanoic acid (stereochemical configuration assumed). Obtained 700 mg (3.89 mmol, 26%) as a white solid. LCMS: m/z=222 [M+1].

### Examples 1 and 2: (2S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (Example 1) and (2R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one (Example 2

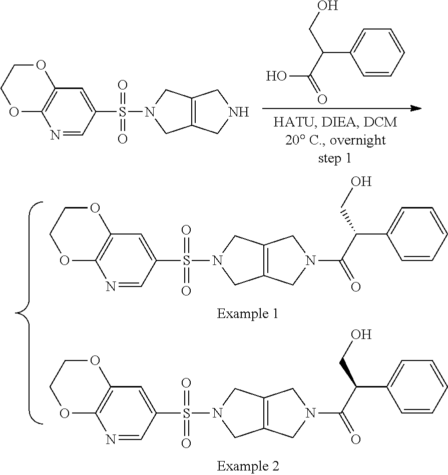 [SMILES: O=S(=O)(c1cnc2c(c1)OCCO2)N1CC2=C(CNC2)C1 ; O=C(O)C(CO)c1ccccc1 ; O=C([C@H](CO)c1ccccc1)N1CC2=C(C1)CN(S(=O)(=O)c1cnc3c(c1)OCCO3)C2 ; O=C([C@@H](CO)c1ccccc1)N1CC2=C(C1)CN(S(=O)(=O)c1cnc3c(c1)OCCO3)C2]

Into a 100 mL round-bottom flask was placed 2-[2H,3H-[1,4]dioxino[2,3-b]pyridine-7-sulfonyl]-1H,2H,3H,4H,5H,6H-pyrrolo[3,4-c]pyrrole (150 mg, 0.48 mmol, 1.00 equiv), 3-hydroxy-2-phenylpropanoic acid (97 mg, 0.58 mmol, 1.20 equiv), dichloromethane (10 mL), HATU (369 mg, 0.97 mmol, 2.00 equiv) and DIEA (188 mg, 1.46 mmol, 3.00 equiv). The resulting solution was stirred overnight at 20° C. The reaction mixture was diluted with 20 mL of water and was then extracted with 3×20 mL of dichloromethane. The organic layers were combined, dried over anhydrous sodium sulfate, filtered and concentrated under vacuum. The residue was purified by prep-TLC eluted with dichloromethane/methanol (20:1) and further purified by prep-HPLC (Column: XBridge C18 OBD Prep Column, 100 Å, 5 μm, 19 mm×250 mm; Mobile Phase A: water (10 mmol/L NH₄HCO₃), Mobile Phase B: MeCN; Gradient: 15% B to 45% B over 8 min; Flow rate: 20 mL/min; UV Detector. 254 nm). And then the two enantiomers were separated by prep-Chiral HPLC (Column, Daicel CHIRALPAK® IF, 2.0 cm×25 cm, 5 μm; mobile phase A: DCM, phase B: MeOH (hold 60% MeOH over 15 min); Flow rate: 16 ml/min; Detector, UV 254 & 220 nm). This resulted in peak 1 (Example 2, Rt: 8.47 min) 9.0 mg (4%) of (2R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one as a yellow solid. And peak 2 (Example 1, Rt: 11.83 min) 10.6 mg (5%) of (2S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-phenylpropan-1-one as a yellow solid.

Example 2 ¹H NMR (400 MHz, DMSO-d₆) δ8.13 (d, J=2.0 Hz, 1H), 7.60 (d, J=2.0 Hz, 1H), 7.31-7.18 (m, 5H), 4.75 (t, J=5.2 Hz, 1H), 4.52-4.45 (m, 2H), 4.40-4.36 (m, 1H), 4.34-4.26 (m, 2H), 4.11-3.87 (m, 8H), 3.80-3.78 (m, 1H), 3.44-3.43 (m, 1H). LC-MS (ESI) m/z: calculated for C₂₂H₂₃N₃O₆S: 457.13; found: 458.0 [M+H]⁺.

Example 1 ¹H NMR (400 MHz, DMSO-d₆) δ8.13 (d, J=2.0 Hz, 1H), 7.61 (d, J=2.0 Hz, 1H), 7.31-7.20 (m, 5H), 4.75 (t, J=5.2 Hz, 1H), 4.50-4.47 (m, 2H), 4.40-4.36 (m, 1H), 4.32-4.29 (m, 2H), 4.11-3.87 (m, 8H), 3.80-3.77 (m, 1H), 3.44-3.41 (m, 1H). LC-MS (ESI) m/z: calculated for C₂₂H₂₃N₃O₆S: 457.13; found: 458.0 [M+H]⁺.

### Examples 3 and 4: (25 or 2R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-(pyridin-2-yl)propan-1-one (Example 4) and (2R or 2S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-(pyridin-2-yl)propan-1-one (Example 3

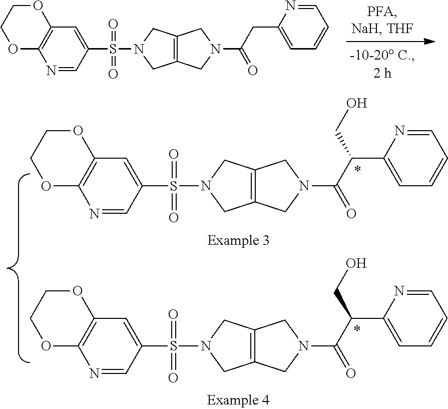 [SMILES: O=C(Cc1ccccn1)N1CC2=C(C1)CN(S(=O)(=O)c1cnc3c(c1)OCCO3)C2 ; O=C([C@H](CO)c1ccccn1)N1CC2=C(C1)CN(S(=O)(=O)c1cnc3c(c1)OCCO3)C2 ; O=C([C@@H](CO)c1ccccn1)N1CC2=C(C1)CN(S(=O)(=O)c1cnc3c(c1)OCCO3)C2]

Into a 25 mL round-bottom flask was placed 1-(5-((2,3-dihydro-[1,4]dioxino[2,3-b]pyridin-7-yl)sulfonyl)-3,4,5,6-tetrahydropyrrolo[3,4-c]pyrrol-2(1H)-yl)-2-(pyridin-2-yl)ethan-1-one (80 mg, 0.17 mmol, 1.00 equiv) and tetrahydrofuran (10 mL). Sodium hydride (60% dispersion in mineral oil, 8 mg, 0.20 mmol, 1.18 equiv) was added. The solution was stirred for 10 min at 20° C., then a solution of paraformaldehyde (8.8 mg) in tetrahydrofuran (1 mL) was added dropwise with stirring at −10° C. The mixture was stirred for 2 h at 20° C., then concentrated under vacuum. The residue was purified by prep-HPLC (Column: XBridge C18 OBD Prep Column, 100 Å, 5 μm, 19 mm×250 mm; Mobile Phase A: water (0.05% NH₃·H₂O), Mobile Phase B: MeCN; Gradient: 20% B to 45% B over 8 min; Flow rate: 20 mL/min; UV Detector. 254 nm). The enantiomers were separated by prep-Chiral HPLC (Column, Daicel CHIRALPAK® ID, 2.0 cm×25 cm, 5 μm; mobile phase A: MeOH, phase B: DCM (hold 30% DCM over 23 min); Detector, Flow rate: 15 ml/min; Detector, UV 254 & 220 nm) to provide (2S or 2R)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-(pyridin-2-yl)propan-1-one as a yellow solid (Example 4, Rt: 12.14 min., 19 mg, 24% yield), and (2R or 2S)-1-(5-[2H,3H-[1,4]dioxino[2,3-b]pyridine-7-sulfonyl]-1H,2H,3H,4H,5H,6H-pyrrolo[3,4-c]pyrrol-2-yl)-3-hydroxy-2-(pyridin-2-yl)propan-1-one as a yellow solid (Example 3, Rt: 18.44 min., 19.3 mg, 25% yield). Absolute stereochemistry was not determined (*).

(Example 4): ¹H NMR (400 MHz, DMSO-d₆): δ 8.45-8.43 (m, 1H), 8.14 (d, J=2.4 Hz, 1H), 7.74-7.62 (m, 1H), 7.62 (d, J=2.4 Hz, 1H), 7.30-7.22 (m, 2H), 4.80 (t, J=5.20 Hz, 1H), 4.50-4.48 (m, 2H), 4.40-4.37 (m, 1H), 4.32-4.30 (m, 2H), 4.05-3.91 (m, 9H), 3.70-3.65 (m, 1H). LC-MS (ESI) m/z: calculated for C₂₁H₂₂N₄O₆S: 458.49; found: 459.0 [M+H]⁺.

(Example 3): ¹H NMR (400 MHz, DMSO-d₆): δ8.45-8.43 (m, 1H), 8.14 (d, J=2.4 Hz, 1H), 7.74-7.62 (m, 1H), 7.61 (d, J=2.4 Hz, 1H), 7.30-7.22 (m, 2H), 4.80 (t, 1=5.2 Hz, 1H), 4.50-4.48 (m, 2H), 4.40-4.37 (m, 1H), 4.32-4.30 (m, 2H), 4.05-3.91 (m, 9H), 3.70-3.65 (m, 1H). LC-MS (ESI) m/z: calculated for C₂₁H₂₂N₄O₆S: 458.49; found: 459.0 [M+H]⁺.

### Examples 5 and 6: (R or S)-(5-((2,3-dihydro-[1,4]dioxino[2,3-b]pyridin-7-yl)sulfonyl)-3,4,5,6-tetrahydropyrrolo[3,4-c]pyrrol-2(1H)-yl)tetrahydro-2H-pyran-3-yl)methanone (Example 5) and (S or R)-(5-((2,3-dihydro-[1,4]dioxino[2,3-b]pyridin-7-yl)sulfonyl)-3,4,5,6-tetrahydropyrrolo[3,4-c]pyrrol-2(1H)-yl)tetrahydro-2H-pyran-3-yl)methanone (Example 6

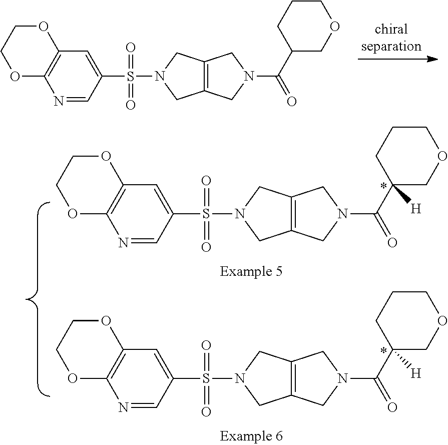 [SMILES: O=C(C1CCCOC1)N1CC2=C(C1)CN(S(=O)(=O)c1cnc3c(c1)OCCO3)C2 ; O=C([C@@H]1CCCOC1)N1CC2=C(C1)CN(S(=O)(=O)c1cnc3c(c1)OCCO3)C2 ; O=C([C@H]1CCCOC1)N1CC2=C(C1)CN(S(=O)(=O)c1cnc3c(c1)OCCO3)C2]

(5-((2,3-Dihydro-[1,4]dioxino[2,3-b]pyridin-7-yl)sulfonyl)-3,4,5,6-tetrahydropyrrolo[3,4-c]pyrrol-2(1H)-yl)tetrahydro-2H-pyran-3-yl)methanone was prepared using the reaction conditions described for Example 1 from the appropriate reagents. The residue was purified by silica gel chromatography eluted with dichloromethane/methanol (20:1) and further purified by prep-HPLC (Column: XBridge BEH C₁₋₈ OBD Prep Column, 130 Å, 5 μm, 19 mm×150 mm; Mobile phase: water (10 mmol NH₄HCO₃), MeCN (1% MeCN up to 40% over 8 min); Flow rate: 20 mL/min; Detector: 254 & 220 nm). The two enantiomers were separated by chiral-prep-HPLC (Column, Daicel CHIRALPAK®1B, 2.0 cm×25 cm, 5 μm; mobile phase A: DCM, phase B: Ethanol (hold 75% DCM over 13 min); Flow rate: 14 ml/min; Detector, UV 254 & 220 nm; Retention time: Example 5: 9.22 min, Example 6: 11.57 min) to provide (R or S)-(5-((2,3-dihydro-[1,4]dioxino[2,3-b]pyridin-7-yl)sulfonyl)-3,4,5,6-tetrahydropyrrolo[3,4-c]pyrrol-2(1H)-yl)(tetrahydro-2H-pyran-3-yl)methanone as a white solid (5.3 mg, 2%) and (S or R)-(5-((2,3-dihydro-[1,4]dioxino[2,3-b]pyridin-7-yl)sulfonyl)-3,4,5,6-tetrahydropyrrolo[3,4-c]pyrrol-2(1H)-yl)(tetrahydro-2H-pyran-3-yl)methanone as a white solid (4.9 mg, 2%). Absolute stereochemistry was not determined (*).

Example 5 ¹H NMR (400 MHz, CDCl₃): δ 8.31 (s, 1H), 7.60 (s, 1H), 4.57-4.50 (m, 2H), 4.36-4.25 (m, 4H), 4.15-4.09 (m, 6H), 3.94-3.88 (m, 2H), 3.56-3.50 (m, 1H), 3.49-3.33 (m, 1H), 2.63-2.60 (m, 1H), 1.95-1.78 (m, 2H), 1.67-1.61 (m, 2H). LC-MS (ESI) m/z: calculated for C₁₉H₂₃N₃O₆S: 421.13; found: 422 [M+H]⁺.

Example 6 ¹H NMR (400 MHz, CDCl₃): δ 8.30 (s, 1H), 7.61 (s, 1H), 4.54-4.52 (m, 2H), 4.35-4.27 (m, 4H), 4.15-4.09 (m, 6H), 3.95-3.90 (m, 2H), 3.56-3.50 (m, 1H), 3.42-3.35 (m, 1H), 2.65-2.60 (m, 1H), 1.95-1.78 (m, 2H), 1.67-1.62 (m, 2H). LC-MS (ESI) m/z: calculated for C₁₉H₂₃N₃O₆S: 421.13; found: 422 [M+H]⁺.

### Example 7: 1-(5-[[4-(Difluoromethoxy)benzene]sulfonyl]-1H,2H,3H,4H,5H,6H-pyrrolo[3,4-c]pyrrol-2-yl)-3-hydroxy-2,2-dimethylpropan-1-one

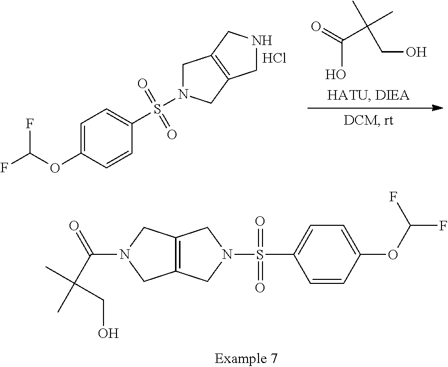

Into a 50-mL round-bottom flask was placed 2-[[4-(difluoromethoxy)benzene]sulfonyl]-1H,2H,3H,4H,5H,6H-pyrrolo[3,4-c]pyrrole hydrochloride (113 mg, 0.32 mmol, 1.00 equiv), dichloromethane (10 mL), 3-hydroxy-2,2-dimethylpropanoic acid (41 mg, 0.35 mmol, 1.10 equiv), DIEA (123 mg, 0.95 mmol, 3.00 equiv) and HATU (241 mg, 0.63 mmol, 2.00 equiv). The solution was stirred for 2 h at room temperature, then concentrated under vacuum. The crude product was purified by Prep-HPLC (Waters I: column: Xbridge Prep C18 5 μm 19×150 mm; mobile phase gradient: CH₃CN/water (0.05% NH₄OH) from 32% to 47% in 7 minute run; detector UV wavelength: 254 nm.) to provide 25.3 mg (19%) of 1-(5-[[4-(difluoromethoxy)benzene]sulfonyl]-1H,2H,3H,4H,5H,6H-pyrrolo[3,4-c]pyrrol-2-yl)-3-hydroxy-2,2-dimethylpropan-1-one as a white solid.

¹H NMR (300 MHz, DMSO-d₆): δ ppm 7.89-7.92 (m, 2H), 7.39-7.42 (d, J=7.8 Hz, 2H), 7.17-7.66 (t, J=73.2 Hz, 1H), 4.68-4.72 (t, J=5.4 Hz, 1H), 3.90-4.50 (m, 8H), 3.40-3.42 (d, J=5.4 Hz, 2H), 1.09 (s, 6H). LC-MS (ESI) m/z: Calculated for C₁₈H₂₂F₂N₂O₅S: 416.12; found: 417 [M+H]⁺.

The Examples in Table 1 below were prepared according to the procedures outlined above for Example 7, using the appropriate synthetic precursors.

TABLE 1

| Example | Structure, Name | LCMS | ¹H NMR |
|---|---|---|---|
| 8 | 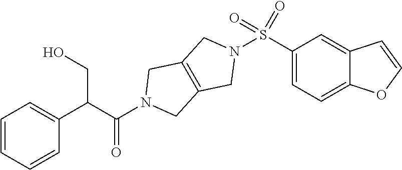 [SMILES: O=C(C(CO)c1ccccc1)N1CC2=C(C1)CN(S(=O)(=O)c1ccc3occc3c1)C2] | m/z: 439 | |
| | 1-(5-(Benzofuran-5-ylsulfonyl)-3,4,5,6- | | |
| | tetrahydropyrrolo[3,4-c]pyrrol-2(1H)-yl)-3- | | |
| | hydroxy-2-phenylpropan-1-one | | |
| 9 | 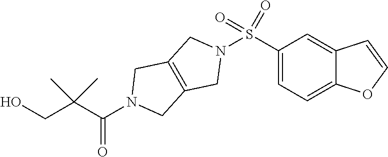 [SMILES: CC(C)(CO)C(=O)N1CC2=C(C1)CN(S(=O)(=O)c1ccc3occc3c1)C2] | m/z: 391 | |
| | 1-(5-(Benzofuran-5-ylsulfonyl)-3,4,5,6- | | |
| | tetrahydropyrrolo[3,4-c]pyrrol-2(1H)-yl)-3- | | |
| | hydroxy-2,2-dimethylpropan-1-one | | |
| 10 | 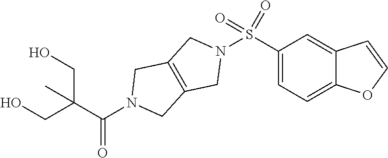 [SMILES: CC(CO)(CO)C(=O)N1CC2=C(C1)CN(S(=O)(=O)c1ccc3occc3c1)C2] | m/z: 407 | |
| | 1-(5-(Benzofuran-5-ylsulfonyl)-3,4,5,6- | | |
| | tetrahydropyrrolo[3,4-c]pyrrol-2(1H)-yl)-3- | | |
| | hydroxy-2-(hydroxymethyl)-2- | | |
| | methylpropan-1-one | | |
| 11 | 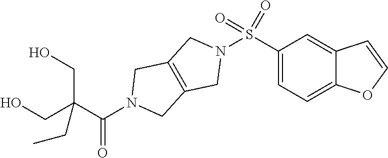 [SMILES: CCC(CO)(CO)C(=O)N1CC2=C(C1)CN(S(=O)(=O)c1ccc3occc3c1)C2] | m/z: 421 | |
| | 1-(5-(Benzofuran-5-ylsulfonyl)-3,4,5,6- | | |
| | tetrahydropyrrolo[3,4-c]pyrrol-2(1H)-yl)- | | |
| | 2,2-bis(hydroxymethyl)butan-1-one | | |
| 12 | 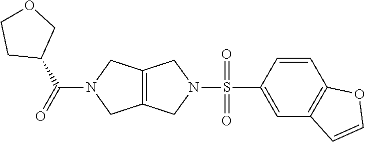 [SMILES: O=C([C@@H]1CCOC1)N1CC2=C(C1)CN(S(=O)(=O)c1ccc3occc3c1)C2] | m/z: 389.1 | |
| | 2-(1-Benzofuran-5-sulfonyl)-5-[(3R)- | | |
| | oxolane-3-carbonyl]-1H,2H,3H,4H,5H,6H- | | |
| | pyrrolo[3,4-c]pyrrole | | |
| 13 | 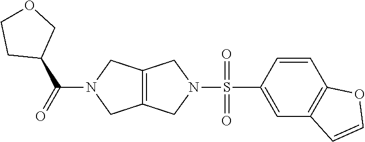 [SMILES: O=C([C@H]1CCOC1)N1CC2=C(C1)CN(S(=O)(=O)c1ccc3occc3c1)C2] | m/z : 389.1 | |
| | 2-(1-Benzofuran-5-sulfonyl)-5-[(3S)- | | |
| | oxolane-3-carbonyl]-1H,2H,3H,4H,5H,6H- | | |
| | pyrrolo[3,4-c]pyrrole | | |
| 14 | 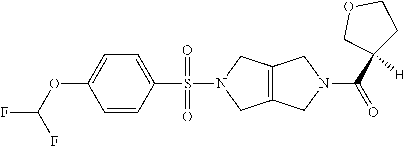 [SMILES: O=C([C@@H]1CCOC1)N1CC2=C(C1)CN(S(=O)(=O)c1ccc(OC(F)F)cc1)C2] | m/z: 415 | (300 MHz, DMSO-d₆): δ ppm 7.89-7.94 (m, 2H),7.40-7.42 (d, 2H), 7.18-7.66 (t, J = 72 Hz, 1H) 3.83-4.29 (m, 9H), 3.62-3.74 (m, 3H), 3.06-3.16(m, 1H), 1.94-2.07 (m, 2H). |
| | (R)-(5-((4- | | |
| | (difluoromethoxy)phenyl)sulfonyl)-3,4,5,6- | | |
| | tetrahydropyrrolo[3,4-c]pyrrol-2(1H)- | | |
| | yl)(tetrahydrofuran-3-yl)methanone | | |
| 15 | 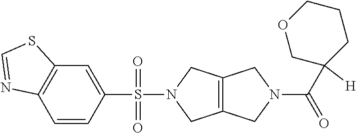 [SMILES: O=C(C1CCCOC1)N1CC2=C(C1)CN(S(=O)(=O)c1ccc3ncsc3c1)C2] | m/z: 420 | (300 MHz, CDCl₃): δ ppm 9.22 (s, 1H), 8.54 (d, J = J = 1.2 Hz, 1H), 8.29 (d,8.7 Hz, 1H), 7.97 (dd, J = 8.7 Hz, J = 1.8 Hz, 1H), 4.24-4.12 (m, 8H), 3.94-3.90 (m, 2H), 3.54-3.41(m, 2H), 2.65-2.55 (m, 1H), |
| | (5-(Benzo[d]thiazol-6-ylsulfonyl)-3,4,5,6- | | 1.86-1.81 (m, 2H), |
| | tetrahydropyrrolo[3,4-c]pyrrol-2(1H)- | | 1.67-1.49 (m, 2H). |
| | yl)(tetrahydro-2H-pyran-3-yl)methanone | | |
| 16 | 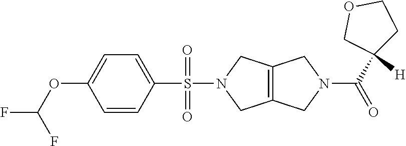 [SMILES: O=C([C@H]1CCOC1)N1CC2=C(C1)CN(S(=O)(=O)c1ccc(OC(F)F)cc1)C2] | m/z: 415 | (300 MHz, DMSO-d₆): δ ppm 7.95-7.98 (m, 2H), 7.35-7.42 (m, 2H), 6.79-7.28 (t, J = 72 Hz, 1H)3.80-4.32 (m, 14H),3.24-3.40 (m, 1H),2.22 (m, 2H). |
| | (S)-(5-((4- | | |
| | (difluoromethoxy)phenyl)sulfonyl)-3,4,5,6- | | |
| | tetrahydropyrrolo[3,4-c]pyrrol-2(1H)- | | |
| | yl)(tetrahydrofuran-3-yl)methanone | | |
| 17 | 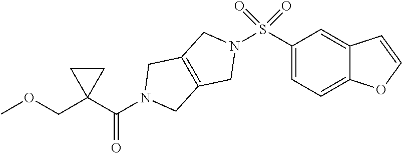 [SMILES: COCC1(C(=O)N2CC3=C(C2)CN(S(=O)(=O)c2ccc4occc4c2)C3)CC1] | m/z: 403 | |
| | (5-(benzofuran-5-ylsulfonyl)-3,4,5,6- | | |
| | tetrahydropyrrolo[3,4-c]pyrrol-2(1H)-yl)(1- | | |
| | (methoxymethyl)cyclopropyl)methanone | | |
| 18 | 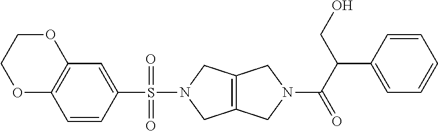 [SMILES: O=C(C(CO)c1ccccc1)N1CC2=C(C1)CN(S(=O)(=O)c1ccc3c(c1)OCCO3)C2] | m/z: 457 | |
| | 1-(5-((2,3-dihydrobenzo[b][1,4]dioxin-6- | | |
| | yl)sulfonyl)-3,4,5,6-tetrahydropyrrolo[3,4-c] | | |
| | pyrrol-2(1H)-yl)-3-hydroxy-2- | | |
| | phenylpropan-1-one | | |

### Example 19: 1-(5-(Benzo[d]thiazol-6-ylsulfonyl)-3,4,5,6-tetrahydropyrrolo[3,4-c]pyrrol-2(1H)-yl)-3-hydroxy-2,2-dimethylpropan-1-one

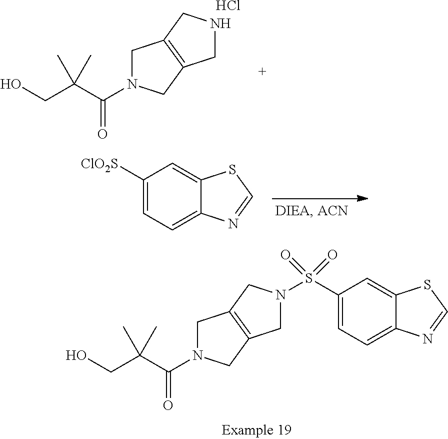

To a 0.2 M solution of 3-hydroxy-2,2-dimethyl-1-(3,4,5,6-tetrahydropyrrolo[3,4-c]pyrrol-2(1H)-yl)propan-1-one hydrochloride (300 μl, 0.060 mmol) in CH₃CN with 10% DIEA was added a 0.2 M solution of benzo[d]thiazole-6-sulfonyl chloride (300 μl, 0.06 mmol) in CH₃CN with 3% DIEA. The reaction was agitated at RT for 5 hours, then concentrated and partitioned between ethyl acetate and aqueous NaOH (1 N). The organic phase was concentrated under reduced pressure and the crude material was purified by prep-HPLC to provide 1-(5-(benzo[d]thiazol-6-ylsulfonyl)-3,4,5,6-tetrahydropyrrolo[3,4-c]pyrrol-2(1H)-yl)-3-hydroxy-2,2-dimethylpropan-1-one (2.6 mg, 10% yield). LCMS: m/z 408 [M+H]⁺.

The Examples in Table 2 below were prepared according to the procedure outlined above for Example 19, using the appropriate synthetic precursors.

TABLE 2

| Example | Structure, Name | LCMS |
|---|---|---|
| 20 | 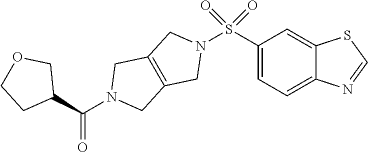 | m/z: 406 |
| | (R)-(5-(benzo[d]thiazol-6-ylsulfonyl)-3,4,5,6-tetrahydropyrrolo[3,4- | |
| | c]pyrrol-2(1H)-yl)(tetrahydrofuran-3-yl)methanone | |
| 21 | 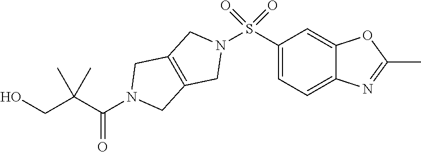 [SMILES: Cc1nc2ccc(S(=O)(=O)N3CC4=C(CN(C(=O)C(C)(C)CO)C4)C3)cc2o1] | m/z: 406 |
| | 3-Hydroxy-2,2-dimethyl-1-(5-((2-methylbenzo[d]oxazol-6-yl)sulfonyl)- | |
| | 3,4,5,6-tetrahydropyrrolo[3,4-c]pyrrol-2(1H)-yl)propan-1-one | |
| 22 | 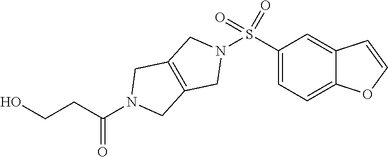 [SMILES: O=C(CCO)N1CC2=C(C1)CN(S(=O)(=O)c1ccc3occc3c1)C2] | m/z: 363 |
| | 1-(5-(Benzofuran-5-ylsulfonyl)-3,4,5,6-tetrahydropyrrolo[3,4-c]pyrrol- | |
| | 2(1H)-yl)-3-hydroxypropan-1-one | |
| 23 | 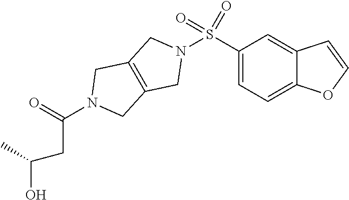 [SMILES: C[C@@H](O)CC(=O)N1CC2=C(C1)CN(S(=O)(=O)c1ccc3occc3c1)C2] | m/z: 377 |
| | (R)-1-(5-(benzofuran-5-ylsulfonyl)-3,4,5,6-tetrahydropyrrolo[3,4- | |
| | c]pyrrol-2(1H)-yl)-3-hydroxybutan-1-one | |
| 24 | 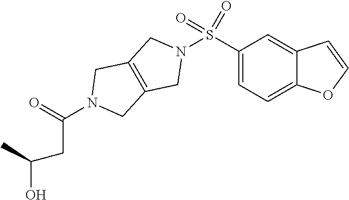 [SMILES: C[C@H](O)CC(=O)N1CC2=C(C1)CN(S(=O)(=O)c1ccc3occc3c1)C2] | m/z: 377 |
| | (S)-1-(5-(benzofuran-5-ylsulfonyl)-3,4,5,6-tetrahydropyrrolo[3,4- | |
| | c]pyrrol-2(1H)-yl)-3-hydroxybutan-1-one | |
| 25 | 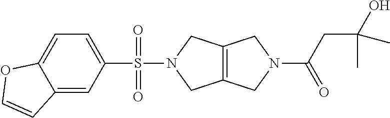 [SMILES: CC(C)(O)CC(=O)N1CC2=C(C1)CN(S(=O)(=O)c1ccc3occc3c1)C2] | m/z: 391.14 |
| | (5-(Benzofuran-5-ylsulfonyl)-3,4,5,6-tetrahydropyrrolo[3,4-c]pyrrol- | |
| | 2(1H)-yl)(1-(methoxymethyl)cyclopropyl)methanone | |

### Example 26: (2S,3R and 2R,3S)-1-(5-[[4-(difluoromethoxy)benzene]sulfonyl]-1H,2H,3H,4H,5H,6H-pyrrolo[3,4-c]pyrrol-2-yl)-3-hydroxy-2-phenylbutan-1-one

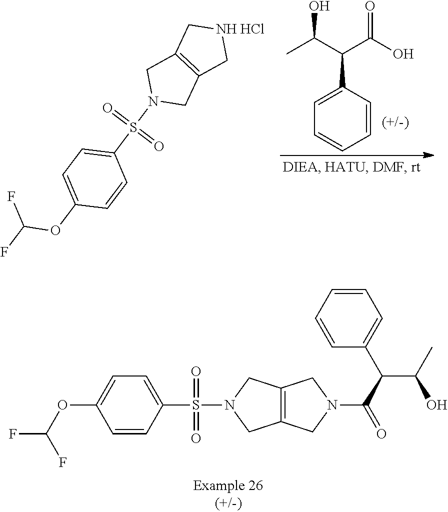

To an 8-mL vial was added 2-((4-(difluoromethoxy)phenyl)sulfonyl)-1,2,3,4,5,6-hexahydropyrrolo[3,4-c]pyrrole hydrochloride (64 mg, 0.18 mmol, 1.00 equiv), DMF (1.50 mL), DIEA (58 mg, 0.45 mmol, 2.50 equiv), (2S,3R and 2R,3S)-3-hydroxy-2-phenylbutanoic acid (40 mg, 0.22 mmol, 1.20 equiv), and HATU (84 mg, 0.22 mmol, 1.20 equiv). The solution was stirred for 16 h at RT. The solution was diluted with 50 mL of ethyl acetate, washed with 4×15 mL of brine, dried over anhydrous sodium sulfate and concentrated under vacuum. The crude product was purified by prep-TLC (DCM/EA=½) to provide (2S,3R and 2R,3S)-1-(5-[[4-(difluoromethoxy)benzene]sulfonyl]-1H,2H,3H,4H,5H,6H-pyrrolo[3,4-c]pyrrol-2-yl)-3-hydroxy-2-phenylbutan-1-one as a white solid (41 mg, 47%). ¹H NMR (300 MHz, CDCl₃): 5 ppm 7.85-7.80 (m, 2H), 7.37-7.23 (m, 7H), 6.59 (t, 1=72.6 Hz, 1H), 4.45-4.37 (m, 1H), 4.25-3.95 (m, 7H), 3.78-3.74 (m, 1H), 3.39 (d, J=3.9 Hz, 1H), 1.05 (d, J=6.3 Hz, 3H). LCMS: m/z=479.0 [M+H]⁺.

The Examples in Table 3 below were prepared according to the procedure outlined above for Example 26, using the appropriate synthetic precursors.

TABLE 3

| Example | Structure, Name | LCMS | ¹H NMR |
|---|---|---|---|
| 27 | 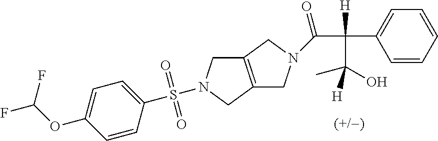 [SMILES: C[C@@H](O)[C@H](C(=O)N1CC2=C(C1)CN(S(=O)(=O)c1ccc(OC(F)F)cc1)C2)c1ccccc1] | m/z: 479 | (300 MHz, CDCl₃): δ ppm 7.84-7.81 (m, 2H), 7.35-7.26 (m, 7H), 6.59 (t, J = 72.6 Hz,1H), 4.35-3.97 (m, 8H), 3.76-3.71 (m, 1H), 3.36-3.34 (m, 1H), 1.06 (dd, J = 13.2 Hz, J = 6.3 Hz, 3H) |
| | (2R,3R and 2S,3S)-1-(5-[[4- | | |
| | (difluoromethoxy)benzene]sulfonyl]- | | |
| | 1H,2H,3H,4H,5H,6H-pyrrolo[3,4-c]pyrrol-2- | | |
| | yl)-3-hydroxy-2-phenylbutan-1-one | | |
| 28 | 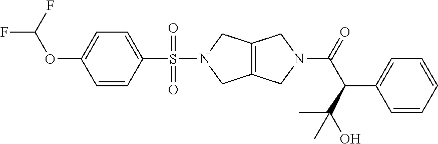 [SMILES: CC(C)(O)[C@@H](C(=O)N1CC2=C(C1)CN(S(=O)(=O)c1ccc(OC(F)F)cc1)C2)c1ccccc1] | m/z: 493 | (300 MHz, CDCl₃): δ ppm 7.83 (d, J = 8.7 Hz, 2H), 7.36-7.23 (m, 7H), 6.59 (t, J = 72.6 Hz, 1H), 4.28-3.85 (m, 8H), 3.32 (s, 1H), 1.39 (s, 3H), 0.92 (s,3H) |
| | (2S)-1-(5-[[4- | | |
| | (difluoromethoxy)benzene]sulfonyl]- | | |
| | 1H,2H,3H,4H,5H,6H-pyrrolo[3,4-c]pyrrol-2- | | |
| | yl)-3-hydroxy-3-methyl-2-phenylbutan-1- | | |
| | one | | |
| 29 | 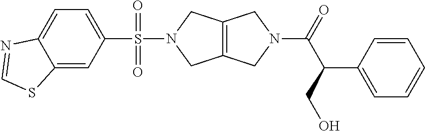 [SMILES: O=C([C@H](CO)c1ccccc1)N1CC2=C(C1)CN(S(=O)(=O)c1ccc3ncsc3c1)C2] | m/z: 456 | (300 MHz, CDCl₃): δ ppm 9.20 (s, 1H), 8.49 (s, 1H), 8.25 (d, J = 6.6 Hz, 1H), 7.94 (d, J = 8.7Hz, 1H), 7.31-7.20 (m, 5H), 4.26-4.03 (m, 8H), 3.73-3.64 (m, 3H) |
| | (2S)-1-[5-(1,3-benzothiazole-6-sulfonyl)- | | |
| | 1H,2H,3H,4H,5H,6H-pyrrolo[3,4-c]pyrrol-2- | | |
| | yl]-3-hydroxy-2-phenylpropan-1-one | | |
| 30 | 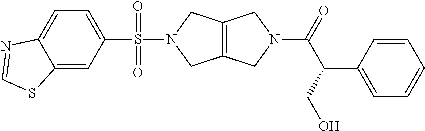 [SMILES: O=C([C@@H](CO)c1ccccc1)N1CC2=C(C1)CN(S(=O)(=O)c1ccc3ncsc3c1)C2] | m/z: 456 | (300 MHz, CDCl₃): δ ppm 9.22 (s, 1H), 8.49 (s, 1H), 8.26 (d, J = 8.4 Hz, 1H), 7.94 (dd, J = 8.4 Hz, J = 1.8 Hz, 1H), 7.34-7.20 (m, 5H), 4.30-4.03 (m,8H), 3.75-3.69 (m, 3H) |
| | (2R)-1-[5-(1,3-benzothiazole-6-sulfonyl)- | | |
| | 1H,2H,3H,4H,5H,6H-pyrrolo[3,4-c]pyrrol-2- | | |
| | yl]-3-hydroxy-2-phenylpropan-1-one | | |
| 31 | 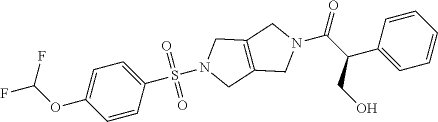 [SMILES: O=C([C@H](CO)c1ccccc1)N1CC2=C(C1)CN(S(=O)(=O)c1ccc(OC(F)F)cc1)C2] | m/z: 465 | (300 MHz, DMSO-d₆): δ ppm 7.89-7.85 (m, 2H), 7.64-7.15 (m, 8H), 4.76 (t, J = 5.1Hz, 1H), 4.40-4.36 (m, 1H), 4.04-3.82 (m, 8H), 3.80-3.77 (m, 1H), 3.48-3.41 (m, 1H) |
| | (S)-1-(5-[[4- | | |
| | (difluoromethoxy)benzene]sulfonyl]- | | |
| | 1H,2H,3H,4H,5H,6H-pyrrolo[3,4-c]pyrrol-2- | | |
| | yl)-3-hydroxy-2-phenylpropan-1-one | | |

### Example 32: (2S)-1-[5-(2,2-dimethyl-3,4-dihydro-2H-1,4-benzoxazine-6-sulfonyl)-1H,2H,3H,4H,5H,6H-pyrrolo[3,4-c]pyrrol-2-yl]-3-hydroxy-2-phenylpropan-1-one

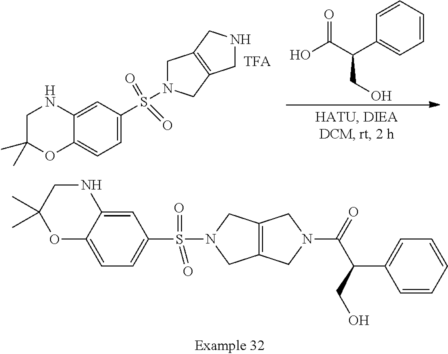

To a 25-mL round-bottom flask was added 2,2-dimethyl-6-[1H,2H,3H,4H,5H,6H-pyrrolo[3,4-c]pyrrole-2-sulfonyl]-3,4-dihydro-2H-1,4-benzoxazine TFA salt (112 mg, 0.25 mmol, 1.00 equiv), (2S)-3-hydroxy-2-phenylpropanoic acid (42 mg, 0.25 mmol, 1.00 equiv), HATU (80 mg, 0.21 mmol, 0.84 equiv), DCM (2.00 mL), and DIEA (58 mg, 0.45 mmol, 2.00 equiv). The solution was stirred for 2 h at 25° C., then extracted with 20 mL of ethyl acetate. The organic phase was washed with 20 mL of brine, dried over anhydrous sodium sulfate, and concentrated under vacuum. The residue was purified by silica gel column chromatography, eluting with dichloromethane/methanol (20/1) to provide (25)-1-[5-(2,2-dimethyl-3,4-dihydro-2H-1,4-benzoxazine-6-sulfonyl)-1H,2H,3H,4H,5H,6H-pyrrolo[3,4-c]pyrrol-2-yl]-3-hydroxy-2-phenylpropan-1-one as a white solid (18.7 mg, 15%). ¹H NMR (300 MHz, DMSO-d₆): δ ppm 7.29-7.28 (m, 5H), 7.04 (s, 1H), 6.90-6.85 (m, 1H), 6.76 (d, J=8.4 Hz, 1H), 6.35 (s, 1H), 4.85-4.70 (m, 1H), 4.50-4.30 (m, 1H), 3.97-3.93 (m, 8H), 3.90-3.80 (m, 1H), 3.35-3.50 (m, 1H), 3.02 (d, J=2.1 Hz, 2H), 1.24 (s, 6H). LCMS: m/z=484.0 [M+H]⁺.

### Examples 33 and 34: 6-(5-[[(3S or 3R)-oxan-3-yl]carbonyl]-1H,2H,3H,4H,5H,6H-pyrrolo[3,4-c]pyrrole-2-sulfonyl)-3,4-dihydro-2H-1,4-benzoxazine (Example 33) and 6-(5-[[(3R or 3S)-oxan-3-yl]carbonyl]-1H,2H,3H,4H,5H,6H-pyrrolo[3,4-c]pyrrole-2-sulfonyl)-3,4-dihydro-2H-1,4-benzoxazine (Example 34

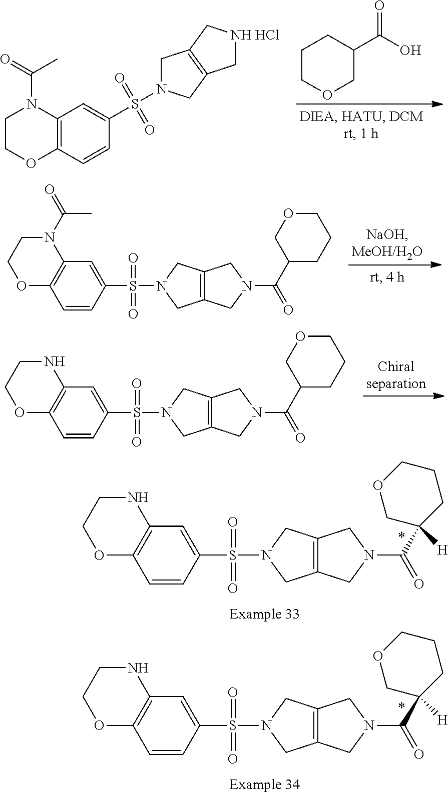

### Step 1. 1-(6-[5-[(Oxan-3-yl)carbonyl]-1H,2H,3H,4H,5H,6H-pyrrolo[3,4-c]pyrrole-2-sulfonyl]-3,4-dihydro-2H-1,4-benzoxazin-4-yl)ethan-1-one

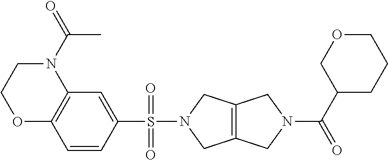 [SMILES: CC(=O)N1CCOc2ccc(S(=O)(=O)N3CC4=C(CN(C(=O)C5CCCOC5)C4)C3)cc21]

Into an 8-mL vial purged and maintained with an inert atmosphere of nitrogen was added oxane-3-carboxylic acid (62.4 mg, 0.48 mmol, 1.20 equiv), DIEA (154.8 mg, 1.20 mmol, 3.00 equiv), 1-(6-((3,4,5,6-tetrahydropyrrolo[3,4-c]pyrrol-2(1H)-yl)sulfonyl)-2,3-dihydro-4H-benzo[b][1,4]oxazin-4-yl)ethan-1-one hydrochloride salt (154.4 mg, 0.40 mmol, 1.00 equiv), HATU (167.2 mg, 0.44 mmol, 1.10 equiv), and dichloromethane (4 ml). The solution was stirred for 4 h at room temperature, then concentrated under vacuum. The crude product was purified by prep-TLC (DCM/MeOH=15/1) to provide 100 mg (54%) of 1-(6-[5-[(oxan-3-yl)carbonyl]-1H,2H,3H,4H,5H,6H-pyrrolo[3,4-c]pyrrole-2-sulfonyl]-3,4-dihydro-2H-1,4-benzoxazin-4-yl)ethan-1-one as a white solid. LCMS (ESI) m/z: Calculated for C₂₂H₂₇N₃O₆S: 461.16; found: 462.0 [M+H]⁺.

### Step 2. 6-[5-[(Oxen-3-yl)carbonyl]-1H,2H,3H,4H,5H,6H-pyrrolo[3,4-c]pyrrole-2-sulfonyl]-3,4-dihydro-2H-1,4-benzoxazine

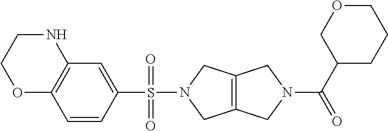 [SMILES: O=C(C1CCCOC1)N1CC2=C(C1)CN(S(=O)(=O)c1ccc3c(c1)NCCO3)C2]

Into an 8-mL vial was placed 1-(6-[5-[(oxan-3-yl)carbonyl]-1H,2H,3H,4H,5H,6H-pyrrolo[3,4-c]pyrrole-2-sulfonyl]-3,4-dihydro-2H-1,4-benzoxazin-4-yl)ethan-1-one (92 mg, 0.20 mmol, 1.00 equiv) and a solution of sodium hydroxide (32 mg, 0.80 mmol, 4.00 equiv) in methanol (2 ml) and water (0.5 ml). The solution was stirred for 4 h at room temperature, then the pH was adjusted to 9 with hydrochloric acid (2 mol/L). The mixture was concentrated under vacuum. The residue was purified by a silica gel column chromatography, eluting with dichloromethane/methanol (50/1). The crude product (100 mg) was further purified by Prep-HPLC (Column: Xbridge Prep C18 5 μm 19×150 mm; mobile phase: water (contains 0.05% NH₃·H₂O) and CH₃CN with a gradient of 16% to 34% CH₃CN in 10 min; detector UV wave length 220 & 254 nm) to provide 80 mg (96%) of 6-[5-[(oxan-3-yl)carbonyl]-1H,2H,3H,4H,5H,6H-pyrrolo[3,4-c]pyrrole-2-sulfonyl]-3,4-dihydro-2H-1,4-benzoxazine as a white solid. LCMS (ESI) m/z: Calculated for C₂₀H₂₅N₃O₅S: 419.15; found: 420 [M+H]⁺.

### Step 3. Examples 33 and 34: 6-(5-[[(3S or 3R)-oxan-3-yl]carbonyl]-1H,2H,3H,4H,5H,6H-pyrrolo[3,4-c]pyrrole-2-sulfonyl)-3,4-dihydro-2H-1,4-benzoxazine and 6-(5-[[(3R or 3S)-oxan-3-yl]carbonyl]-1H,2H,3H,4H,5H,6H-pyrrolo[3,4-c]pyrrole-2-sulfonyl)-3,4-dihydro-2H-1,4-benzoxazine

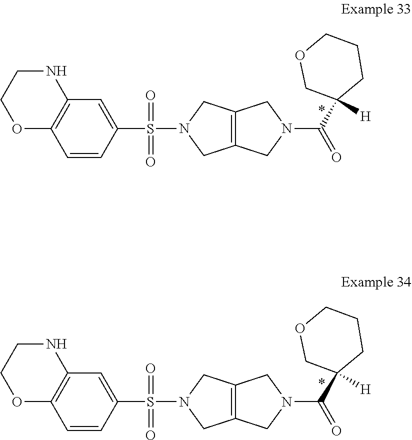 [SMILES: O=C([C@H]1CCCOC1)N1CC2=C(C1)CN(S(=O)(=O)c1ccc3c(c1)NCCO3)C2 ; O=C([C@@H]1CCCOC1)N1CC2=C(C1)CN(S(=O)(=O)c1ccc3c(c1)NCCO3)C2]

Chiral separation of racemic 6-[5-[(oxan-3-yl)carbonyl]-1H,2H,3H,4H,5H,6H-pyrrolo[3,4-c]pyrrole-2-sulfonyl]-3,4-dihydro-2H-1,4-benzoxazine (80 mg) was carried out by Chiral-Prep-HPLC (SHIMADZU LC-20AT: Column, CHIRALPAK IC; mobile phase, A: Ethanol [containing 0.1% DEA], Phase B: Methanol; detector UV wave length: 220 nm) to provide 22.3 mg (28%) of 6-(5-[[(3S or 3R)-oxan-3-yl]carbonyl]-1H,2H,3H,4H,5H,6H-pyrrolo[3,4-c]pyrrole-2-sulfonyl)-3,4-dihydro-2H-1,4-benzoxazine (Example 33) as a white solid, and 18.9 mg (24%) of 6-(5-[[(3S or 3R)-oxan-3-yl]carbonyl]-1H, 2H, 3H, 4H, 5H, 6H-pyrrolo[3,4-c]pyrrole-2-sulfonyl)-3,4-dihydro-2H-1,4-benzoxazine (Example 34) as a white solid. Absolute stereochemistry was not determined (*).

Example 33. Prep chiral HPLC Rt=24.2 min. ¹H NMR (300 MHz, CDCl₃): 5 ppm 7.24-7.19 (m, 2H), 6.89 (d, J=6.6 Hz, 1H), 4.44-4.34 (d, 2H), 4.25-4.18 (m, 2H), 4.12 (s, 6H), 3.95-3.91 (m, 2H), 3.56-3.37 (m, 4H), 2.66-2.62 (m, 1H), 1.89-1.68 (m, 4H). LC-MS (ESI) m/z: Calculated for C₂₀H₂₅N₃O₅S: 419.15; found: 420 [M+H]⁺.

Example 34 Prep chiral HPLC Rt=30.4 min. ¹H NMR (300 MHz, CDCl₃): δ ppm 7.17-7.11 (m, 2H), 6.88 (d, J=8.1 Hz, 1H), 4.33-4.25 (m, 4H), 4.12 (s, 6H), 3.95-3.91 (m, 2H), 3.56-3.37 (m, 4H), 2.67-2.57 (m, 1H), 1.89-1.66 (m, 4H). LC-MS (ESI) m/z: Calculated for C₂₀H₂₅N₃O₅S: 419.15; found: 420 [M+H]⁺.

The Examples in Table 4 below were prepared according to the procedures outlined above for Example 33 and 34, steps 1 and 2, using the appropriate synthetic precursors.

TABLE 4

| Example | Structure, Name | LCMS |
|---|---|---|
| 35 | 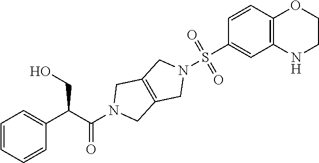 [SMILES: O=C([C@H](CO)c1ccccc1)N1CC2=C(C1)CN(S(=O)(=O)c1ccc3c(c1)NCCO3)C2] | m/z: 456 |
| | (S)-1-(5-((3,4-dihydro-2H-benzo[b][1,4]oxazin-6-yl)sulfonyl)-3,4,5,6- | |
| | tetrahydropyrrolo[3,4-c]pyrrol-2(1H)-yl)-3-hydroxy-2-phenylpropan-1-one | |
| 36 | 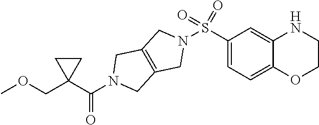 [SMILES: COCC1(C(=O)N2CC3=C(C2)CN(S(=O)(=O)c2ccc4c(c2)NCCO4)C3)CC1] | m/z: 420 |
| | (5-((3,4-Dihydro-2H-benzo[b][1,4]oxazin-6-yl)sulfonyl)-3,4,5,6- | |
| | tetrahydropyrrolo[3,4-c]pyrrol-2(1H)-yl)(1- | |
| | (methoxymethyl)cyclopropyl)methanone | |
| 37 | 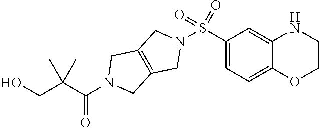 [SMILES: CC(C)(CO)C(=O)N1CC2=C(C1)CN(S(=O)(=O)c1ccc3c(c1)NCCO3)C2] | m/z: 408 |
| | 1-(5-((3,4-Dihydro-2H-benzo[b][1,4]oxazin-6-yl)sulfonyl)-3,4,5,6- | |
| | tetrahydropyrrolo[3,4-c]pyrrol-2(1H)-yl)-3-hydroxy-2,2-dimethylpropan- | |
| | 1-one | |
| 38 | 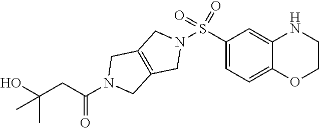 [SMILES: CC(C)(O)CC(=O)N1CC2=C(C1)CN(S(=O)(=O)c1ccc3c(c1)NCCO3)C2] | m/z: 408 |
| | 1-(5-((3,4-Dihydro-2H-benzo[b][1,4]oxazin-6-yl)sulfonyl)-3,4,5,6- | |
| | tetrahydropyrrolo[3,4-c]pyrrol-2(1H)-yl)-3-hydroxy-3-methylbutan-1-one | |
| 39 | 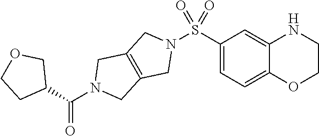 | m/z: 406 |
| | (R)-(5-((3,4-dihydro-2H-benzo[b][1,4]oxazin-6-yl)sulfonyl)-3,4,5,6- | |
| | tetrahydropyrrolo[3,4-c]pyrrol-2(1H)-yl)(tetrahydrofuran-3-yl)methanone | |

### Examples 41 and 42: (S or R-)-1-(5-(4-(Difluoromethoxy)phenylsulfonyl)-4,5-dihydropyrrolo[3,4-c]pyrrol-2(1H,3H,4H)-yl)-3-hydroxy-2-(pyridin-2-yl)propan-1-one (Example 41) and (R or S)-1-(5-(4-(difluoromethoxy)phenylsulfonyl)-4,5-dihydropyrrolo[3,4-c]pyrrol-2(1H,3H,4H)-yl)-3-hydroxy-2-(pyridin-2-yl)propan-1-one (Example 42

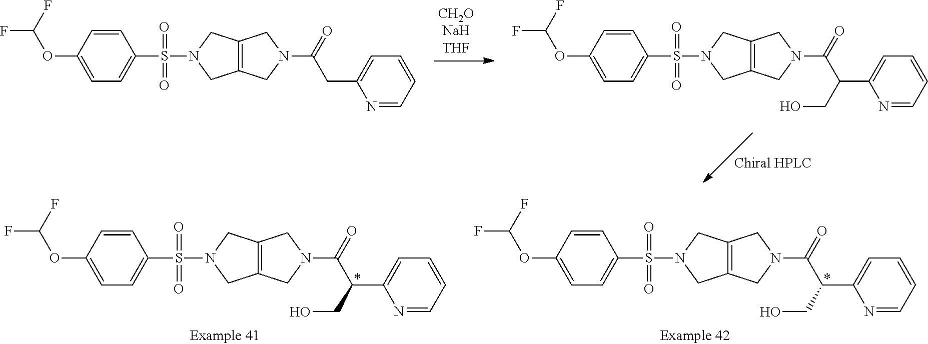 [SMILES: O=C(Cc1ccccn1)N1CC2=C(C1)CN(S(=O)(=O)c1ccc(OC(F)F)cc1)C2 ; O=C(C(CO)c1ccccn1)N1CC2=C(C1)CN(S(=O)(=O)c1ccc(OC(F)F)cc1)C2 ; O=C([C@H](CO)c1ccccn1)N1CC2=C(C1)CN(S(=O)(=O)c1ccc(OC(F)F)cc1)C2 ; O=C([C@@H](CO)c1ccccn1)N1CC2=C(C1)CN(S(=O)(=O)c1ccc(OC(F)F)cc1)C2]

To a 100-mL 3-necked round-bottom flask was added 1-(5-[[4-(difluoromethoxy)benzene]sulfonyl]-1H,2H,3H,4H,5H,6H-pyrrolo[3,4-c]pyrrol-2-yl)-2-(pyridin-2-yl)ethan-1-one hydrochloride salt (80 mg, 0.18 mmol, 1.00 equiv) and sodium hydride (60% oil dispersion, 4.4 mg, 0.18 mmol, 1.00 equiv) in tetrahydrofuran (10 mL). The reaction mixture was cooled down to −10° C. and formaldehyde (5.5 mg, 0.18 mmol, 1.00 equiv, 0.2 mL in THF) was added dropwise.

The mixture was stirred for 4 hours at 25° C., then quenched by addition of water (20 mL). The solution was extracted with dichloromethane (3×20 mL). The combined organic layers were evaporated under reduced pressure and the crude material was purified by Prep-HPLC with the following conditions: Column, X-bridge RP18, 5 μm, 19×150 mm; mobile phase: water (it contains 0.03% ammonia) and CH₃CN with a gradient of 45% to 60% CH₃CN in 5 min; flow rate: 20 mL/min; detector UV wavelength: 254 nm. This provided racemic 1-(5-(4-(difluoromethoxy)phenylsulfonyl)-4,5-dihydropyrrolo[3,4-c]pyrrol-2(1H,3H,4H)-yl)-3-hydroxy-2-(pyridin-2-yl)propan-1-one (76 mg, 89%) as a white solid. The enantiomers were separated by Chiral-Prep-HPLC (SHIMADZU LC-20AD) with the following conditions: Column, DAICEL chiral PAK OD-H, 20×250 mm, 5 μm; mobile phase: Phase A: ethanol, Phase B: methanol (containing 0.1% DEA) with isocratic elution of 60% ethanol; flow rate: 15 mL/min; detector wavelength: 220 nm. Absolute stereochemistry was not determined (*). This provided:

### Example 41: (S or R)-1-(5-(4-(Difluoromethoxy)phenylsulfonyl)-4,5-dihydropyrrolo[3,4-c]pyrrol-2(1H,3H,4H)-yl)-3-hydroxy-2-(pyridin-2-yl)propan-1-one

Isolated as a yellow solid (11.3 mg, 15%). ¹H NMR (300 MHz, DMSO-d₆): δ ppm 8.45-8.47 (m, 1H), 7.90-7.87 (m, 2H), 7.70-7.75 (m, 1H), 7.37 (t, J=73.2 Hz, 1H), 7.23-7.37 (m, 4H), 4.70-4.85 (m, 1H), 4.37-4.42 (m, 1H), 4.03-4.06 (m, 9H), 3.70-3.72 (m, 1H). LCMS: m/z=466 [M+H]⁺.

### Example 42: (R or S)-1-(5-(4-(Difluoromethoxy)phenylsulfonyl)-4,5-dihydropyrrolo[3,4-c]pyrrol-2(1H,3H,4H)-yl)-3-hydroxy-2-(pyridin-2-yl)propan-1-one

Isolated as a yellow solid (14.2 mg, 19%). ¹H NMR (300 MHz, DMSO-d₆): δ ppm 8.45-8.47 (m, 1H), 7.90-7.87 (m, 2H), 7.70-7.75 (m, 1H), 7.31 (t, 1=73.2 Hz, l H), 7.23-7.31 (m, 4H), 4.70-4.85 (m, 1H), 4.38-4.42 (m, 1H), 4.03-4.06 (m, 9H), 3.69-3.72 (m, 1H). LCMS: m/z=466 [M+H]⁺.

### Example 43: (5-(Benzo[d]thiazol-6-ylsulfonyl)-3,4,5,6-tetrahydropyrrolo[3,4-c]pyrrol-2(1H)-yl)(2,3-dihydrobenzofuran-3-yl)methanone

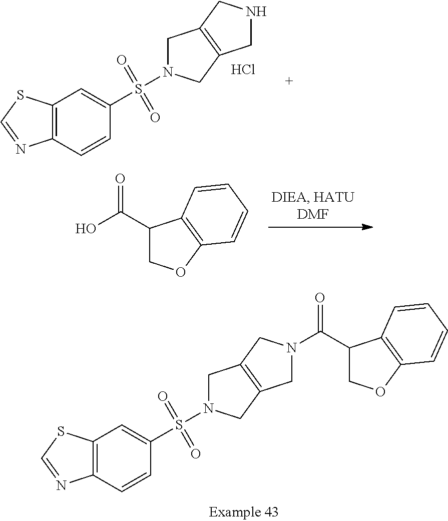

To an 8-mL vial, purged and maintained with an inert atmosphere of nitrogen, was added 6-((3,4,5,6-tetrahydropyrrolo[3,4-c]pyrrol-2(1H)-yl)sulfonyl)benzo[d]thiazole hydrochloride (50 mg, 0.15 mmol, 1.00 equiv), 2,3-dihydro-1-benzofuran-3-carboxylic acid (29 mg, 0.18 mmol, 1.20 equiv), DIEA (68 mg, 0.53 mmol, 3.50 equiv), HATU (65 mg, 0.17 mmol, 1.20 equiv), and DMF (1.00 mL). The solution was stirred for 16 h at RT. Water (2 mL) was added dropwise. The solids were collected by filtration. The filter cake was washed with H₂O (0.5 mL) and MeOH (1.0 mL), and the filtrate was collected and dried under vacuum to provide (5-(benzo[d]thiazol-6-ylsulfonyl)-3,4,5,6-tetrahydropyrrolo[3,4-c]pyrrol-2(1H)-yl)(2,3-dihydrobenzofuran-3-yl)methanone (30 mg, 45%) as an off-white solid. ¹H NMR (300 MHz, DMSO-d₆): δ ppm 9.66 (s, 1H), 8.84 (d, J=1.5 Hz, 1H), 8.32 (d, J=8.4 Hz, 1H), 7.99 (dd, J1=8.7 Hz, 12=1.8 Hz, 1H), 7.14-7.09 (m, 2H), 6.80-6.75 (m, 2H), 4.67-4.38 (m, 5H), 4.18 (s, 4H), 4.01 (m, 2H). LCMS: m/z=454 [M+H]⁺.

### Examples 44 and 45: (R or S)-(5-(pyridin-2-ylsulfonyl)-3,4,5,6-tetrahydropyrrolo[3,45]pyrrol-2(1H)-yl)(tetrahydro-2H-pyran-3-yl)methanone (Example 44) and (S or R)-(5-(pyridin-2-ylsulfonyl)-3,4,5,6-tetrahydropyrrolo[3,4-c]pyrrol-2(1H)-yl)(tetrahydro-2H-pyran-3-yl)methanone (Example 45

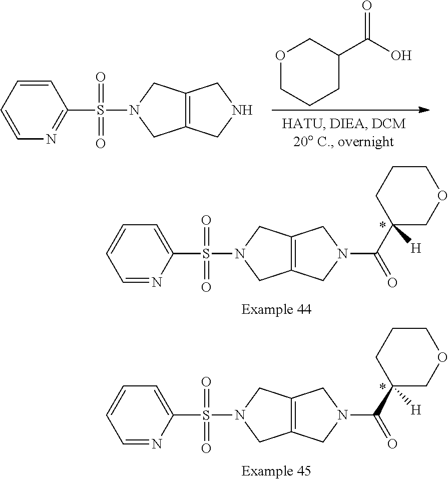

To a 50-mL round-bottom flask was added 2-(pyridin-2-ylsulfonyl)-1,2,3,4,5,6-hexahydropyrrolo[3,4-c]pyrrole (100 mg, 0.40 mmol, 1.00 equiv), oxane-3-carboxylic acid (52 mg, 0.40 mmol, 1.00 equiv), HATU (302 mg, 0.79 mmol, 1.97 equiv), DCM (10 mL), and DIEA (154 mg, 1.19 mmol, 2.99 equiv). The solution was stirred overnight at 20° C. The mixture was diluted with 20 mL of DCM, washed with 2×20 mL of water, dried over anhydrous sodium sulfate, filtered, and concentrated under vacuum. The residue was purified by silica gel column chromatography, eluting with ethyl acetate/petroleum ether (10/1). The enantiomers were separated by prep-Chiral HPLC with the following conditions: column, Daicel CHIRALPAK® IA 21.2×250 mm, 5 μm; mobile phase, A=Hexane, phase B=EtOH (hold 50.0% EtOH over 42 min); flow rate, 20 mL/min; Detector, UV 254 & 220 nm. Absolute stereochemistry was not determined (*). This provided:

### Example 44. (R or S)-(5-(pyridin-2-ylsulfonyl)-3,4,5,6-tetrahydropyrrolo[3,4-c]pyrrol-2(1H)-yl)(tetrahydro-2H-pyran-3-yl)methanone (stereochemical configuration assumed

Isolated as a white solid (12.1 mg, 8%). Prep-Chiral HPLC Rt=24.472 min. ¹H NMR (400 MHz, CDCl₃): δ 8.73-8.69 (m, 1H), 8.03-7.88 (m, 2H), 7.56-7.42 (m, 1H), 4.43-4.26 (m, 6H), 4.16 (d, J=3.6 Hz, 2H), 3.98-3.87 (m, 2H), 3.54 (t, J=12.0 Hz, 1H), 3.50-3.34 (m, 1H), 2.68-2.49 (m, 1H), 1.96-1.76 (m, 2H), 1.69-1.48 (m, 2H). LCMS: m/z=364.0 [M+H]⁺.

### Example 45. (S or R)-(5-(pyridin-2-ylsulfonyl)-3,4,5,6-tetrahydropyrrolo[3,4-c]pyrrol-2(1H)-yl)(tetrahydro-2H-pyran-3-yl)methanone (stereochemical configuration assumed

Isolated as a white solid (7.3 mg, 5%). Prep-Chiral HPLC Rt=33.498 min. ¹H NMR (400 MHz, CDCl₃): δ 8.75-8.67 (m, 1H), 8.04-7.88 (m, 2H), 7.58-7.39 (m, 1H), 4.43-4.26 (m, 6H), 4.18-4.16 (m, 2H), 4.00-3.89 (m, 2H), 3.54 (t, J=12.0 Hz, 1H), 3.48-3.29 (m, 1H), 2.69-2.48 (m, 1H), 1.95-1.76 (m, 2H), 1.72-1.58 (m, 2H). LCMS: m/z=364.2 [M+H]⁺.

### Example 46: 3-Hydroxy-1-(5-((4-methyl-3,4-dihydro-2H-benzo[b][1,4]oxazin-6-yl)sulfonyl)-3,4,5,6-tetrahydropyrrolo[3,4-c]pyrrol-2(1H)-yl)-2-phenylpropan-1-one

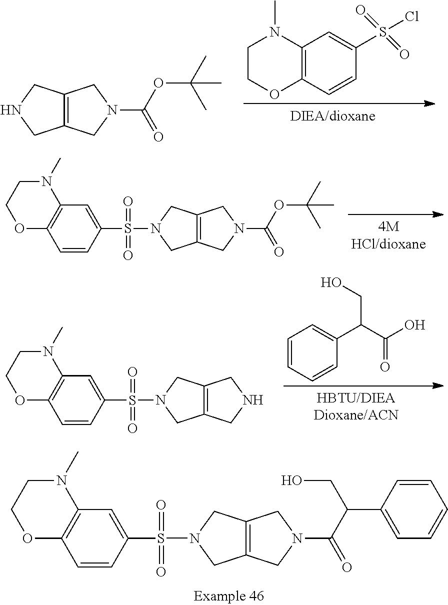

To a 1.5 mL vial was added a 0.2 M solution of tert-butyl 3,4,5,6-tetrahydropyrrolo[3,4-c]pyrrole-2(1H)-carboxylate hydrochloride (100 μL, 20 μmol) in dioxane and neat DIEA (10 μL, 57 μmol) to give a brown suspension. A 0.2 M solution of 4-methyl-3,4-dihydro-2H-benzo[b][1,4]oxazine-6-sulfonyl chloride (105 μL, 20 μmol) in dioxane was added. The reaction was heated at 50° C. with shaking for 2 hours. 4 M HCl in dioxane (50.0 μL, 0.200 mmol) was then added. The reaction was heated at 50° C. with shaking for an additional 2 hours. The volatiles were removed under reduced pressure. ACN (200 μL) was added to the vial. The vial was shaken for 15 minutes to resuspend the residue. Neat DIEA (25 μL, 0.143 mmol) and a 0.2 M solution of 3-hydroxy-2-phenylpropanoic acid (110 μL, 22 μmol) in dioxane was added to the vial, followed by a 0.2 M solution of HBTU (110 μL, 22 μmol) in ACN. The reaction was heated at 50° C. with shaking for an additional 2 hours. The volatiles were removed under reduced pressure. The residue was mixed with 1 N NaOH (0.5 mL) and extracted with 3:1 EtOAc/ACN (2×0.5 mL). The volatiles were removed under reduced pressure. The compound was purified using mass-triggered HPLC to give 3-hydroxy-1-(5-((4-methyl-3,4-dihydro-2H-benzo[b][1,4]oxazin-6-yl)sulfonyl)-3,4,5,6-tetrahydropyrrolo[3,4-c]pyrrol-2(1H)-yl)-2-phenylpropan-1-one. LCMS: m/z=470.2 [M+H]⁺.

The Examples in Table 5 below were prepared according to the procedure outlined above for Example 46, using the appropriate synthetic precursors.

TABLE 5

| Example | Name | Structure | LCMS |
|---|---|---|---|
| 40 | 3-hydroxy-1-(5-((4- methoxyphenyl)sulfonyl)-3,4,5,6- tetrahydropyrrolo[3,4-c]pyrrol- 2(1H)-yl)propan-1-one | 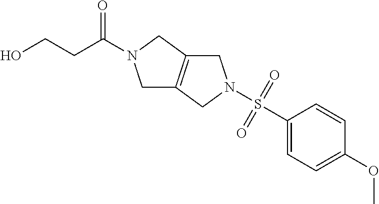 [SMILES: COc1ccc(S(=O)(=O)N2CC3=C(CN(C(=O)CCO)C3)C2)cc1] | |
| 47 | 4-Methyl-6-{[5-(oxolane-3- carbonyl)-1H,2H,3H,4H,5H,6H- pyrrolo[3,4-c]pyrrol-2-yl]sulfonyl}- 3,4-dihydro-2H-1,4-benzoxazine | 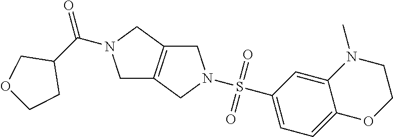 [SMILES: CN1CCOc2ccc(S(=O)(=O)N3CC4=C(CN(C(=O)C5CCOC5)C4)C3)cc21] | m/z: 420.2 |
| 48 | 1-[5-(1,3-Benzothiazole-6-sulfonyl)- 1H,2H,3H,4H,5H,6H-pyrrolo[3,4- c]pyrrol-2-yl]-3-hydroxy-2- phenylpropan-1-one | 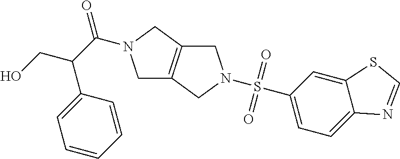 [SMILES: O=C(C(CO)c1ccccc1)N1CC2=C(C1)CN(S(=O)(=O)c1ccc3ncsc3c1)C2] | m/z: 456.1 |
| 49 | 1-{5-[4- (Difluoromethoxy)benzenesulfonyl]- 1H,2H,3H,4H,5H,6H-pyrrolo[3,4- c]pyrrol-2-yl}-3-hydroxy-2- phenylpropan-1-one | 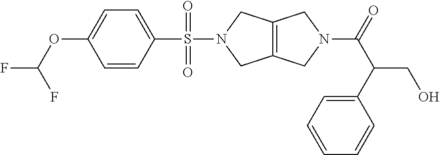 [SMILES: O=C(C(CO)c1ccccc1)N1CC2=C(C1)CN(S(=O)(=O)c1ccc(OC(F)F)cc1)C2] | m/z: 465.1 |
| 50 | (2S)-3-hydroxy-2-phenyl-1-[5- (pyridine-3-sulfonyl)- 1H,2H,3H,4H,5H,6H-pyrrolo[3,4- c]pyrrol-2-yl]propan-1-one | 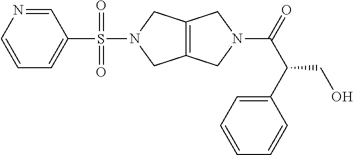 [SMILES: O=C([C@H](CO)c1ccccc1)N1CC2=C(C1)CN(S(=O)(=O)c1cccnc1)C2] | m/z: 400.3 |
| 51 | (2S)-3-hydroxy-2-phenyl-1-[5- (pyridine-2-sulfonyl)- 1H,2H,3H,4H,5H,6H-pyrrolo[3,4- c]pyrrol-2-yl]propan-1-one | 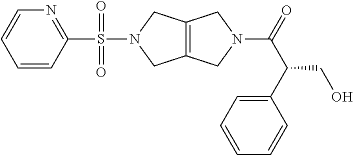 [SMILES: O=C([C@H](CO)c1ccccc1)N1CC2=C(C1)CN(S(=O)(=O)c1ccccn1)C2] | m/z: 400.3 |
| 52 | (2S)-3-hydroxy-2-phenyl-1-(5-{[6- (trifluoromethyl)pyridin-3- yl]sulfonyl}-1H,2H,3H,4H,5H,6H- pyrrolo[3,4-c]pyrrol-2-yl)propan-1- one | 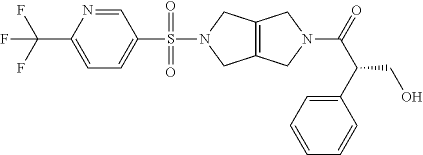 [SMILES: O=C([C@H](CO)c1ccccc1)N1CC2=C(C1)CN(S(=O)(=O)c1ccc(C(F)(F)F)nc1)C2] | m/z: 468.2 |
| 53 | 3-Methoxy-1-{5-[(4-methyl-3,4- dihydro-2H-1,4-benzoxazin-6- yl)sulfonyl]-1H,2H,3H,4H,5H,6H- pyrrolo[3,4-c]pyrrol-2-yl}propan-1- one | 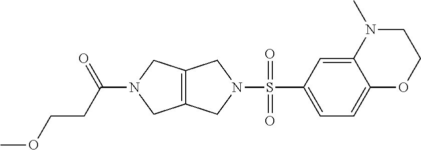 [SMILES: COCCC(=O)N1CC2=C(C1)CN(S(=O)(=O)c1ccc3c(c1)N(C)CCO3)C2] | m/z: 408.2 |
| 54 | 1-{5-[4- (Difluoromethoxy)benzenesulfonyl]- 1H,2H,3H,4H,5H,6H-pyrrolo[3,4- c]pyrrol-2-yl}-3-hydroxypropan-1- one | 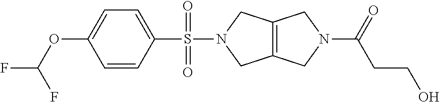 [SMILES: O=C(CCO)N1CC2=C(C1)CN(S(=O)(=O)c1ccc(OC(F)F)cc1)C2] | m/z: 391.1 |
| 55 | (5-(benzofuran-5-ylsulfonyl)- 3,4,5,6-pyrrolo[3,4-c]pyrrol-2(1H)- yl)(tetrahydrofuran-3-yl)methanone | 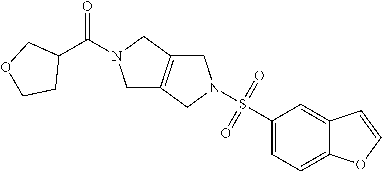 [SMILES: O=C(C1CCOC1)N1CC2=C(C1)CN(S(=O)(=O)c1ccc3occc3c1)C2] | |
| 56^{a} | (5-(benzo[d]thiazol-6-ylsulfonyl)- 3,4,5,6-tetrahydropyrrolo[3,4- c]pyrrol-2(1H)-yl)(morpholin-3- yl)methanone | 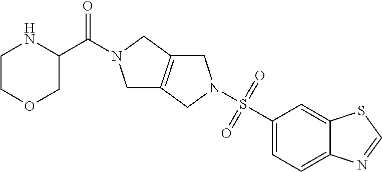 [SMILES: O=C(C1COCCN1)N1CC2=C(C1)CN(S(=O)(=O)c1ccc3ncsc3c1)C2] | |
| 57 | 1-(5-((2,3- dihydrobenzo[b][1,4]dioxin-6- yl)sulfonyl)-3,4,5,6- tetrahydropyrrolo[3,4-c]pyrrol- 2(1H)-yl)-3-methoxypropan-1-one | 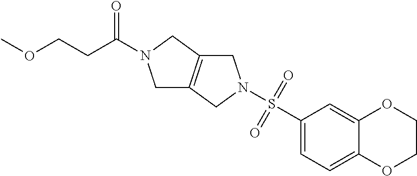 [SMILES: COCCC(=O)N1CC2=C(C1)CN(S(=O)(=O)c1ccc3c(c1)OCCO3)C2] | |
| 58 | 1-(5-(benzofuran-5-ylsulfonyl)- 3,4,5,6-tetrahydropyrrolo[3,4- c]pyrrol-2(1H)-yl)-3- methoxypropan-1-one | 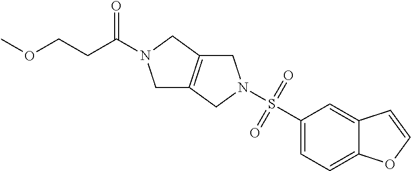 [SMILES: COCCC(=O)N1CC2=C(C1)CN(S(=O)(=O)c1ccc3occc3c1)C2] | |

^{a}The morpholine moiety was protected with a Boc group on nitrogen throughout the synthesis of this molecule. The final step of the synthesis was deprotection of the Boc group (see step 2 in Example 46).

### Example 47: PKR(wt/without FBP), PKR(G332S/with FBP), PKR(R510Q/without FBP

### Luminescence Assay Protocol

In some embodiments, a “PKR Activating Compound” refers to a compound having one or more characteristics when tested according to the following Luminescence Assay Protocol of Example 47 performed with wild type (wt) PKR and/or any one or more of G332S mutant form of PKR or R510Q mutant form of PKR: (1) an AC₅₀ value of less than 40 μM (e.g., compounds with AC₅₀ values of “+”, “++”, or “+++” in Table 6); (2) a maximum % Fold (MAX % Fold) value of greater than 75%; and/or (3) a % Fold value at 1.54 μM compound concentration (% Fold@1.54 μM) of at least 75%. In some embodiments, a PKR Activating Compound can have: (1) an AC₅₀ value of less than 0.1 μM (e.g., compounds with AC₅₀ values of “+++” in Table 6), 0.1-1.0 μM (e.g., compounds with AC₅₀ values of “++” in Table 6), or 1.01-40 μM (e.g., compounds with AC₅₀ values of “+” in Table 6); (2) a MAX % Fold of 75%-250%, 250-500%, or 75%-500%; and/or (3) a % Fold@1.54 μM of 75%-250%, 250-500%, or 75%-500%. In some embodiments, a PKR Activating Compound has (1) an AC₅₀ value of less than 1.0 μM; (2) a MAX % Fold of 75%-500%; and/or (3) a % Fold@1.54 μM of 75%-500%, obtained in the Luminescence Assay Protocol with any one or more of wild type PKR (wt), G332S mutant form of PKR, or R510Q mutant form of PKR. In some embodiments, the PKR Activating Compound has (1) an AC₅₀ value of less than 1.0 μM; (2) a MAX % Fold of 75%-500%; and/or (3) a % Fold@1.54 μM of 75%-500%, obtained in the Luminescence Assay Protocol with wild type PKR (wt). In some embodiments, the PKR Activating Compound has (1) an AC₅₀ value of less than 1.0 μM; (2) a MAX % Fold of 75%-500%; and/or (3) a % Fold@1.54 μM of 75%-500%, obtained in the Luminescence Assay Protocol with any one or both of G332S mutant form of PKR or R510Q mutant form of PKR.

The phosphorylation of Adenosine-5′-diphosphate (ADP) by various mutants of PKR was determined by the Kinase Glo Plus Assay (Promega) in the presence or absence of FBP [D-Fructose-1,6-diphosphate; BOC Sciences, CAS: 81028-91-3] as follows. Unless otherwise indicated, all reagents were purchased from Sigma-Aldrich. All reagents were prepared in buffer containing 50 mM Tris-HCl, 100 mM KCl, 5 mM MgCl₂, and 0.01% Triton X100, 0.03% BSA, and 1 mM DTT. Enzyme and PEP [Phospho(enol) pyruvic acid] were added at 2× to all wells of an assay-ready plate containing serial dilutions of test compounds or DMSO vehicle. Final enzyme concentrations for PKR(wt), PKR(R510Q), and PKR(G332S) were 0.8 nM, 0.8 nM, and 10 nM respectively. Final PEP concentration was 100 μM. The Enzyme/PEP mixture was incubated with compounds for 30 minutes at RT before the assay was initiated with the addition of 2× ADP [Adenosine-5′-diphosphate] and KinaseGloPlus. Final concentration of ADP was 100 μM. Final concentration of KinaseGloPlus was 12.5%. For assays containing FBP, that reagent is added at 30 μM upon reaction initiation. Reactions were allowed to progress for 45 minutes at RT until luminescence was recorded by the BMG PHERAstar FS Multilabel Reader. All compounds were tested in triplicate at concentrations ranging from 42.5 μM to 2.2 nM in 0.83% DMSO.

Luminescence values were converted to % Fold increase by normalizing to the average of the DMSO control and multiplying by 100. Max, min, slope and AC₅₀ were determined by the standard four parameter fit algorithm of ActivityBase XE Runner. Compounds were evaluated with three parameters —AC₅₀, MAX % Fold, and % Fold@1.54 μM (FIG. 1). The AC₅₀ value for a compound is the concentration (μM) corresponding to the midway between the maximum and minimum values of the four parameter logistic curve fit (i.e., at which the % fold increase along the four parameter logistic curve fit is halfway between MAX % Fold and MIN % Fold (% Fold Midpoint)), MAX % Fold is the highest fold increase observed at any concentration of compound, and % Fold@1.54 μM is the fold increase at a compound concentration of 1.54 μM. The parameter % Fold@1.54 μM was selected to capture elements of both the AC₅₀ and MAX % Fold and to provide a ranking based on both potency and effect. The compound concentration of 1.54 μM was chosen as one that can optimally differentiate the set of compounds based on the range of activities observed.

As set forth in Tables 6 and 7 below, AC₅₀ values (columns A, D, G) are defined as follows: ≤0.1 μM (+++); >0.1 μM and ≤1.0 μM (++); >1.0 μM and ≤40 μM (+); >40 μM (0). Max % FOLD values (columns B, E, H) are defined as follows: ≤75% (+); >75% and ≤250% (++); >250% and ≤500% (+++). % Fold@1.54 μM values (columns C, F, 1) are defined as follows: ≤75% (+); >75% and ≤250% (++); >250% and ≤500% (+++).

TABLE 6
Biological Data

| | PKRG332S | | | PKRR510Q | | | WT | | |
|---|---|---|---|---|---|---|---|---|---|
| | Conditions¹ | | | Conditions¹ | | | Conditions¹ | | |
| Example | A | B | C | D | E | F | G | H | I |
| 1 | ++ | ++ | ++ | +++ | +++ | +++ | +++ | ++ | ++ |
| 2 | + | ++ | ++ | + | +++ | ++ | + | ++ | ++ |
| 3^{a} | + | ++ | ++ | ++ | +++ | ++ | +++ | ++ | ++ |
| 4^{a} | | | | + | ++ | ++ | + | ++ | ++ |
| 5^{b} | + | ++ | ++ | + | +++ | ++ | ++ | ++ | ++ |
| 6^{b} | 0 | | | 0 | | | + | ++ | ++ |
| 7 | +++ | | | ++ | | | ++ | ++ | ++ |
| 8 | | | | | | | +++ | +++ | +++ |
| 9 | | | | | | | +++ | ++ | ++ |
| 10 | | | | | | | +++ | +++ | ++ |
| 11 | | | | | | | ++ | +++ | ++ |
| 12 | | | | | | | ++ | +++ | +++ |
| 13 | | | | | | | ++ | +++ | ++ |
| 14 | ++ | ++ | ++ | + | +++ | ++ | ++ | +++ | +++ |
| 15 | 0 | ++ | ++ | + | +++ | ++ | ++ | ++ | ++ |
| 16 | ++ | ++ | ++ | + | +++ | ++ | ++ | +++ | ++ |
| 17 | | | | | | | ++ | ++ | ++ |
| 18 | 0 | ++ | ++ | +++ | ++ | ++ | | | |
| 19 | 0 | ++ | ++ | ++ | +++ | ++ | ++ | ++ | ++ |
| 20 | ++ | ++ | ++ | + | +++ | ++ | ++ | +++ | ++ |
| 21 | | | | | | | + | ++ | ++ |
| 22 | | | | | | | ++ | +++ | +++ |
| 23 | | | | | | | ++ | +++ | +++ |
| 24 | | | | | | | ++ | +++ | ++ |
| 25 | | | | | | | ++ | +++ | ++ |
| 26 | +++ | ++ | ++ | +++ | +++ | +++ | +++ | +++ | +++ |
| 27 | +++ | ++ | ++ | ++ | +++ | +++ | +++ | +++ | +++ |
| 28 | +++ | ++ | ++ | + | +++ | ++ | ++ | +++ | ++ |
| 29 | +++ | ++ | ++ | +++ | +++ | +++ | +++ | +++ | +++ |
| 30 | ++ | ++ | ++ | ++ | +++ | +++ | ++ | +++ | +++ |
| 31 | +++ | ++ | ++ | +++ | +++ | +++ | +++ | +++ | +++ |
| 32 | 0 | ++ | ++ | +++ | ++ | ++ | +++ | ++ | ++ |
| 33^{c} | 0 | ++ | ++ | + | +++ | ++ | + | ++ | ++ |
| 34^{c} | ++ | ++ | ++ | +++ | +++ | +++ | +++ | ++ | ++ |
| 35 | +++ | ++ | ++ | +++ | +++ | +++ | +++ | ++ | ++ |
| 36 | 0 | ++ | ++ | + | ++ | ++ | ++ | ++ | ++ |
| 37 | 0 | ++ | ++ | +++ | +++ | +++ | ++ | ++ | ++ |
| 38 | | | | | | | | | |
| 39 | +++ | ++ | ++ | ++ | +++ | +++ | ++ | +++ | +++ |
| 40 | | | | + | +++ | ++ | | | |
| 41^{d} | +++ | ++ | ++ | +++ | +++ | +++ | +++ | +++ | +++ |
| 42^{d} | +++ | ++ | ++ | +++ | +++ | +++ | +++ | +++ | +++ |
| 43 | ++ | ++ | ++ | + | +++ | +++ | ++ | +++ | +++ |
| 44^{e} | + | ++ | ++ | 0 | ++ | ++ | + | ++ | ++ |
| 45^{e} | ++ | ++ | ++ | + | +++ | ++ | ++ | +++ | ++ |
| 46 | 0 | ++ | ++ | 0 | ++ | ++ | +++ | ++ | ++ |
| 47 | 0 | ++ | ++ | + | +++ | ++ | ++ | ++ | ++ |
| 48 | +++ | ++ | ++ | +++ | +++ | +++ | +++ | +++ | +++ |
| 49 | +++ | ++ | ++ | +++ | +++ | +++ | +++ | +++ | +++ |
| 50 | ++ | ++ | ++ | ++ | +++ | +++ | ++ | +++ | ++ |
| 51 | ++ | ++ | ++ | +++ | +++ | +++ | +++ | +++ | +++ |
| 52 | ++ | ++ | ++ | + | +++ | ++ | ++ | ++ | ++ |
| 53 | 0 | ++ | ++ | + | ++ | ++ | ++ | ++ | ++ |
| 54 | | | | + | +++ | ++ | + | +++ | ++ |
| 55 | | | | | | | | | |
| 56 | 0 | ++ | ++ | + | ++ | ++ | | | |
| 57 | +++ | ++ | ++ | ++ | ++ | ++ | | | |
| 58 | +++ | ++ | ++ | ++ | +++ | ++ | | | |

^{a}Compounds 3 and 4 are enantiomers, but absolute stereochemistry is undetermined;
^{b}Compounds 5 and 6 are enantiomers, but absolute stereochemistry is undetermined;
^{c}Compounds 33 and 34 are enantiomers, but absolute stereochemistry is undetermined;
^{d}Compounds 41 and 42 arc enantiomers, but absolute stereochemistry is undetermined;
^{e}Compounds 44 and 45 are enantiomers, but absolute stereochemistry is undetermined.
¹A - AC50 LUM KGP FBP AC₅₀ μM gmean;
B - AC50 LUM KGP FBP MAX % FOLD mean;
C - AC50 LUM KGP FBP % Fold @ 1.54 μM mean
D - AC50 LUM KGP woFBP AC₅₀ μM gmean;
E - AC50 LUM KGP woFBP MAX % FOLD mean;
F - AC50 LUM KGP woFBP % Fold @ 1.54 μM mean
G - AC50 LUM KGP woFBP AC₅₀ μM gmean;
H - AC50 LUM KGP woFBP MAX % FOLD mean;
I - AC50 LUM KGP woFBP % Fold @ 1.54 μM mean.

TABLE 7
Biological Data of Additional Compounds.

| | | PKRG332S | PKRR510Q |
|---|---|---|---|
| | | Conditions¹ | |
| Example | Structure | A | D |
| 59 | 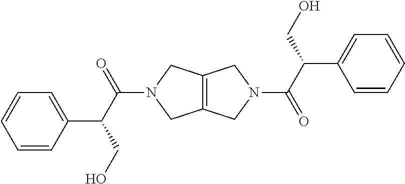 [SMILES: O=C([C@H](CO)c1ccccc1)N1CC2=C(C1)CN(C(=O)[C@H](CO)c1ccccc1)C2] | 0 | 0 |
| 60 | 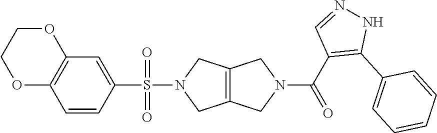 [SMILES: O=C(c1cn[nH]c1-c1ccccc1)N1CC2=C(C1)CN(S(=O)(=O)c1ccc3c(c1)OCCO3)C2] | 0 | 0 |

¹A-AC50 LUM KGP FBP AC₅₀ μM gmean; D-AC50 LUM KGP woFBP AC₅₀ μM gmean.

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of formula 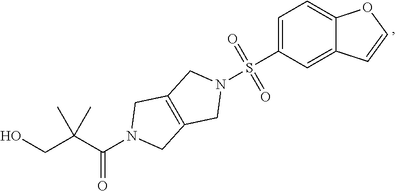 [SMILES: CC(C)(CO)C(=O)N1CC2=C(C1)CN(S(=O)(=O)c1ccc3occc3c1)C2]
or a pharmaceutically acceptable salt thereof.

2. A compound of formula

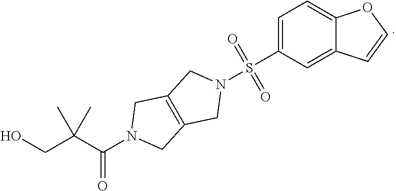 [SMILES: CC(C)(CO)C(=O)N1CC2=C(C1)CN(S(=O)(=O)c1ccc3occc3c1)C2]

3. A pharmaceutical composition comprising the compound of claim 1, or a pharmaceutically acceptable salt thereof, and a pharmaceutically acceptable carrier.

4. A pharmaceutical composition comprising the compound of claim 2, and a pharmaceutically acceptable carrier.
